(19) United States
(12) Patent Application Publication
(10) Pub. No.: US 2019/0125672 A1
(43) Pub. Date: May 2, 2019

(54) Fabrication of Magnetic Vesicles for Biomedical Imaging and Delivery

(71) Applicant: University of Maryland, College Park, College Park, MD (US)

(72) Inventors: Zhihong NIE, Bethesda, MD (US); Yijing LIU, College Park, MD (US); Kuikun YANG, Lanham, MD (US)

(73) Assignee: University of Maryland, College Park, College Park, MD (US)

(21) Appl. No.: 16/173,822

(22) Filed: Oct. 29, 2018

Related U.S. Application Data
(60) Provisional application No. 62/578,259, filed on Oct. 27, 2017.

(51) Int. Cl. A61K 9/127; A61K 47/02; A61K 47/32; A61K 31/65; A61K 49/18; A61K 49/22; A61K 41/00; A61P 35/00

(52) U.S. Cl. CPC A61K 9/1273; A61K 47/02; A61K 47/32; A61P 35/00; A61K 49/1812; A61K 49/227; A61K 41/00; A61K 31/65

(57) ABSTRACT

The present invention is directed to compositions useful in assembling vesicles. The composition comprises a first block copolymer; a plurality of first inorganic nanoparticles; a second block copolymer; and a plurality of second inorganic nanoparticles or a plurality of small molecules. The composition is characterized by the ability to self-assemble into a vesicle. Also provided is a method of making a composition for delivery of a therapeutic agent and a method of using the vesicles as imaging agents.

### STATEMENT REGARDING FEDERALLY SPONSORED RESEARCH OR DEVELOPMENT

This invention was made with government support under DMR 1255377 and CHE 1505839 awarded by the National Science Foundation (NSF). The government has certain rights to this invention.

### BACKGROUND OF THE INVENTION

### Field of the Invention

The present invention is directed to compositions useful in assembling vesicles. The composition comprises a first block copolymer; a plurality of first inorganic nanoparticles; a second block copolymer; and a plurality of second inorganic nanoparticles or a plurality of small molecules. The composition is characterized by the ability to self-assemble into a vesicle. Also provided is a method of making a composition for delivery of a therapeutic agent and a method of using the vesicles as imaging agents.

### BACKGROUND

Inorganic nanoparticles (NPs) have shown promising applications in the treatment, diagnosis, and detection of many diseases, due to their unique optical or magnetic properties. For this purpose, single NPs are often functionalized with organic or polymeric ligands to improve their stability, biocompatibility, and targeted delivery of therapeutic agents. While single NPs are attractive, NP assemblies can exhibit new or advanced properties that are different from those of individual NPs, thus facilitating their biomedical applications. One typical example is vesicular structures containing both NPs and polymers in the membrane. For instance, vesicular assemblies of AuNPs can be used for effective encapsulation of therapeutic agents, near-infrared (NIR) light-triggered release of payload, and multimodality imaging of cancers. The embedding of magnetic nanoparticles (MNPs) in polymeric vesicular membranes increases the stability and biocompatibility of MNPs in physiological environment. Moreover, the presence of many MNPs within individual assemblies increases their responsivness to external magnetic field and transverse relaxivity (r₂). The strong magnetic responsiveness promotes the accumulation of NPs in tumors by application of a magnetic field, thus overcoming the limitation of tumor heterogeneity on passive tumor accumulation of NPs.

Superparamagnetic iron oxide nanoparticles (SPIONs) have been widely explored for biomedical applications, such as biosensing, immunoassays, cell separation, and cancer imaging and therapy, due to their unique size, biocompatibility, biostability, and responsiveness to magnetic field. For instance, SPIONs can serve as negative magnetic resonance imaging (MM) contrast agents, as they can shorten the transverse relaxation time (T₂) of water protons, resulting in a hypointense signal in T₂-weighted Mill. The magnetic movement of SPION-based nanocarriers can be used to guide the delivery of therapeutic agents specifically to diseased areas to achieve optimal therapy outcomes. However, small SPIONs inherently possess a relatively low magnetization per particle, making it difficult to readily manipulate their movement in relatively deep tissues. Increasing the size of iron oxide nanoparticles (NPs) (e.g., above ˜26 nm) leads to a higher magnetic moment, but at the expense of inducing a superparamagnetic/ferromagnetic transition and hence possible colloidal instability of NPs.

Nanosized vesicles (e.g., liposomes or polymersomes) are particularly attractive and have made the greatest clinical impact, because of their unique ability to encapsulate and deliver hydrophilic and/or hydrophobic compounds simultaneously. Incorporating SPIONs into organic vesicular membranes can impart the system with magneto-responsiveness in order to develop highly selective and effective therapeutics and diagnostics. One commonly used strategy for the fabrication of SPION-embedded nanovesicles is to co-assemble hydrophobic small molecular ligand-covered SPIONs with amphiphilic lipids or block copolymers (BCPs). During the assembly, SPIONs are inserted into the hydrophobic domains (e.g., center of lipid bilayers) of vesicular membranes through hydrophobic interaction between capping agents and hydrophobic segments of lipids or BCPs. Small NPs (<8 nm) are usually used in the fabrication, in order to avoid possible insertion-induced morphological change or hole formation of vesicles. More recently, the assembly of BCP-tethered NPs has provided an effective route to the fabrication of hybrid vesicles with high density and much broader size range of NPs in the membrane. These hybrid vesicles have been demonstrated for enhancing MM and photoacoustic imaging, as well as efficacy in photothermal/photodynamic therapy due to their collective properties of assembled NPs.

The present invention provides a fabrication method for magnetic vesicles integrated with metal nanoparticles and magnetic nanoparticles. The present invention also provides a fabrication method for magnetic vesicles (MVs) comprising tunable layers of densely-packed superparamagnetic iron oxide nanoparticles (SPIONs) in membranes. The vesicles are made entirely through self-assembly and templating techniques, which are cost-effective and scalable to large areas.

### BRIEF SUMMARY OF THE INVENTION

The present disclosure provides a composition comprising:

(a) a first block copolymer comprising at least two polymer blocks, wherein at least one of the polymer blocks has been functionalized;

(b) a plurality of first inorganic nanoparticles bound to the surface of the first block copolymer;

(c) a second block copolymer comprising at least two polymer blocks; and

(d) a plurality of second inorganic nanoparticles; or

(a′) a first block copolymer comprising at least two polymer blocks, wherein at least one of the polymer blocks has been functionalized;

(b′) a plurality of small molecules bound to the surface of the first block copolymer;

(c′) a second block copolymer comprising at least two polymer blocks; and

(d′) a plurality of inorganic nanoparticles, wherein the plurality of small

molecules are bound to the surface of the inorganic nanoparticles; wherein the composition is in the form of vesicles.

In some embodiments, the first block copolymer in (a) or (a′) comprises a first polymer block and a second polymer block.

In some embodiments, the first polymer block is polystyrene.

In some embodiments, the second polymer block is poly(ethylene oxide).

In some embodiments, the second block copolymer in (c) or (c′) comprises a first polymer block and a second polymer block.

In some embodiments, the first polymer block is polystyrene.

In some embodiments, the second polymer block in poly(acrylic acid).

In some embodiments, the composition comprises:
(a) a first block copolymer comprising at least two polymer blocks, wherein at least one of the polymer blocks has been functionalized;
(b) a plurality of first inorganic nanoparticles bound to the surface of the first block copolymer;
(c) a second block copolymer comprising at least two polymer blocks; and
(d) a plurality of second inorganic nanoparticles;
   wherein the composition is in the form of vesicles.

In some embodiments, the first inorganic nanoparticles comprise Au.

In some embodiments, the second inorganic nanoparticles comprise iron oxide.

In some embodiments, the first block copolymer comprises a first polymer block comprising polystyrene and a second polymer block comprising poly(ethylene oxide), the first inorganic nanoparticles comprise Au having a diameter of from 20 nm to 50 nm, the second block copolymer comprises a first polymer block comprising polystyrene and a second polymer block comprising poly(acrylic acid), and the second inorganic nanoparticles comprise iron oxide.

In some embodiments, the composition comprises:
(a′) a first block copolymer comprising at least two polymer blocks, wherein at least one of the polymer blocks has been functionalized;
(b′) a plurality of small molecules bound to the surface of the first block copolymer;
(c′) a second block copolymer comprising at least two polymer blocks; and
(d′) a plurality of inorganic nanoparticles, wherein the plurality of small molecules are bound to the surface of the inorganic nanoparticles;
   wherein the composition is in the form of vesicles.

In some embodiments, the small molecule comprises dopamine.

In some embodiments, the inorganic nanoparticles comprise iron oxide.

In some embodiments, the first block copolymer comprises a first polymer block comprising polystyrene and a second polymer block comprising poly(ethylene oxide), the small molecule is dopamine, the second polymer block copolymer comprises a first block comprising polystyrene and a second polymer block comprising poly(acrylic acid), and the inorganic nanoparticles comprise iron oxide.

In some embodiments, the vesicles have a size range of 10 nm to 1000 nm.

In some embodiments, the composition further comprises a therapeutic agent.

In some embodiments, the therapeutic agent comprises doxorubicin.

In some embodiments, the transverse relaxivity (r₂) of the formed vesicles is between about 150 mM⁻¹ s⁻¹ to about 300 mM⁻¹s⁻¹.

In some embodiments, the present disclosure provides a method of making a composition for delivery of a therapeutic agent, the method comprising:
(i) providing a composition in the form of vesicles comprising:
   (a) a first block copolymer comprising at least two polymer blocks, wherein at least one of the polymer blocks has been functionalized;
   (b) a plurality of first inorganic nanoparticles bound to the surface of the first block copolymer;
   (c) a second block copolymer comprising at least two polymer blocks; and
   (d) a plurality of second inorganic nanoparticles; or
   (a′) a first block copolymer comprising at least two polymer blocks, wherein at least one of the polymer blocks has been functionalized;
   (b′) a plurality of small molecules bound to the surface of the first block copolymer;
   (c′) a second block copolymer comprising at least two polymer blocks; and
   (d′) a plurality of inorganic nanoparticles, wherein the plurality of small molecules are bound to the surface of the inorganic nanoparticles; and
(ii) contacting the composition of (a) with a solution containing the therapeutic agent to be delivered and forming vesicles comprising the therapeutic agent encapsulated in the vesicles, thereby forming a composition in the form of vesicles for the delivery of the therapeutic agent.

The present disclosure also provides a method of imaging a biological target, the method comprising:
(i) providing a composition in the form of vesicles comprising:
   (a) a first block copolymer comprising at least two polymer blocks, wherein at least one of the polymer blocks has been functionalized;
   (b) a plurality of first inorganic nanoparticles bound to the surface of the first block copolymer;
   (c) a second block copolymer comprising at least two polymer blocks; and
   (d) a plurality of second inorganic nanoparticles; or
   (a′) a first block copolymer comprising at least two polymer blocks, wherein at least one of the polymer blocks has been functionalized;
   (b′) a plurality of small molecules bound to the surface of the first block copolymer;
   (c′) a second block copolymer comprising at least two polymer blocks; and
   (d′) a plurality of inorganic nanoparticles, wherein the plurality of small molecules are bound to the surface of the inorganic nanoparticles; and
(ii) detecting the vesicles.

In some embodiments, detecting the vesicles uses one or more of a fluorescence microscope, laser-confocal microscopy, cross-polarization microscopy, nuclear scintigraphy, positron emission tomography, single photon emission computed tomography, magnetic resonance imaging, photoacoustic imaging, magnetic resonance spectroscopy, computed tomography, or a combination thereof

In some embodiments, the formed vesicles in (i) have a transverse relaxivity (r₂) between about 150 mM⁻¹s⁻¹ to about 300 mM⁻¹s⁻¹.

### BRIEF DESCRIPTION OF DRAWINGS

FIG. 1 is a schematic illustrating the self-assembly of a ternary mixture of magnetic nanoparticles (MNPs) and free block copolymers (BCPs) of polystyrene-b-poly(acrylic acid) (PS-b-PAA) and polystyrene-b-poly(ethylene oxide) (PS-b-PEO) tethered gold nanoparticles (AuNPs) into hybrid Janus vesicles (JVs) with different morphologies: a spherical Janus vesicle and a hemispherical Janus vesicle.

FIG. 2A is a bar graph of the transverse relaxivity (r₂) of a hemispherical Janus vesicle and a spherical Janus vesicle. As shown in FIG. 2A the hemispherical Janus vesicle has a higher transverse relaxivity than the spherical Janus vesicle.

FIG. 2B is a graph of the near infrared absorption of a hemispherical Janus vesicle.

FIG. 3 is a schematic of the external magnetic field-enhanced magnetic resonance (MR) and photoacoustic (PA) imaging of a tumor after intravenous injection of hemispherical Janus vesicles.

FIG. 4A is a scanning electron microscope (SEM) image of 20 nm AuNPs.

The scale bar represents 200 nm.

FIG. 4B is a SEM image of 30 nm AuNPs. The scale bar represents 200 nm.

FIG. 5A is a transmission electron microscope (TEM) image of 50 nm

AuNPs. The scale bar represents 200 nm.

FIG. 5B is a TEM of 15 nm Fe₃O₄ nanoparticles (NPs). The scale bar represents 20 nm.

FIG. 6A is a SEM image of magneto-plasmonic Janus vesicles with spherical shapes. The inset in FIG. 6A is a TEM image of magneto-plasmonic Janus vesicles with spherical shapes. The mass fraction of MNPs (25 nm) used in self-assembly was 5.8 weight percent. The scale bar represents 500 nm.

FIG. 6B is a SEM image of magneto-plasmonic Janus vesicles with hemspherical shapes. The inset in FIG. 6B is a TEM image of magneto-plasmonic Janus vesicles with hemispherical shapes. The mass fraction of MNPs (25 nm) used in self-assembly was 11.0 weight percent. The scale bar represents 500 nm.

FIG. 7A is an energy-dispersive X-ray spectroscopy (EDS) image of Fe and Au in the spherical Janus vesicles. The scale bars represent 200 nm.

FIG. 7B is an EDS image of Fe and Au in the hemispherical Janus vesicles. The scale bars represent 300 nm.

FIG. 7C is a graph of the formation of hybrid vesicles with different morphologies attained by variation of the core size of BCP-tethered AuNPs and mass fraction of MNPs. Spherical homogeneous vesicles are represented by □, spherical Janus vesicles are represented by ∘, and hemispherical Janus vesicles are represented by Δ.

FIG. 8A is a TEM image of spherical Janus vesicles prepared using BCP-tethered AuNPs (50 nm AuNPs), 15 nm MNPs, and PS₁₀₇-b-PAA₄. The scale bar represents 500 nm.

FIG. 8B is a TEM image of spherical Janus vesicles prepared using BCP-tethered AuNPs (50 nm AuNPs), 15 nm MNPs, and PS₁₀₇-b-PAA₄. The scale bar represents 500 nm.

FIG. 9A is a TEM image of hemispherical Janus vesicles prepared using BCP-tethered AuNPs (20 nm AuNPs), 15 nm MNPs, and PS₁₀₇-b-PAA₄. The scale bar represents 500 nm.

FIG. 9B is a SEM image of hemispherical Janus vesicles prepared using BCP-tethered AuNPs (20 nm AuNPs), 15 nm MNPs, and PS₁₀₇-b-PAA₄. The scale bar represents 500 nm.

FIG. 10A is a SEM image of spherical homogeneous vesicles (HVs) prepared using BCP-tethered AuNPs (20 nm AuNPs), 15 nm MNPs, and PS₁₀₇-b-PAA₄. The scale bar represents 500 nm.

FIG. 10B is a TEM image of spherical homogeneous vesicles (HVs) prepared using BCP-tethered AuNPs (20 nm AuNPs), 15 nm MNPs, and PS₁₀₇-b-PAA₄. The scale bar represents 200 nm.

FIG. 11A is a line graph of the dynamic light scattering data of spherical Janus vesicles prepared using BCP-tethered AuNPs (50 nm AuNPs), 15 nm MNPs, and PS₁₀₇-b-PAA₄. The average diameter of the hybrid vesicles is estimated to be 570±93.2 nm.

FIG. 11B is a high angle annular dark field scanning transmission electron microscope (STEM) of the spherical Janus vesicles prepared using BCP-tethered AuNPs (50 nm AuNPs), 25 nm MNPs, and PS₁₀₇-b-PAA₄.

FIG. 12A is a graph of the UV/Vis absorption spectra of 20 nm AuNPs ( [symbol] ), 50 nm AuNPs ( - - - ), spherical Janus vesicles with 50 nm AuNPs ( - ● -), hemispherical Janus vesicles with 50 nm AuNPs ( - - - - ), and hemispherical Janus vesicles with 20 nm AuNPs ( ● ● ● ● ).

FIG. 12B is a graph of the transverse relaxivity (r₂) of single 15 nm MNPs (□), spherical Janus vesicles (Δ), and hemispherical Janus vesicles (∘) composed of 50 nm AuNPs and 15 nm MNPs. The initial mass fractions of MNPs used in the assembly process were 2.5 weight percent for spherical Janus vesicles and 11.0 weight percent for hemispherical Janus vesicles. The inset in FIG. 12B shows the corresponding T₂-weighted images for single MNPs (top), spherical Janus vesicles (middle), and hemispherical Janus vesicles (bottom). Concentrations of Fe are: 0, 0.031, 0.063, and 0.12 mM (from left to right).

FIG. 13A is a graph of the photothermal heating induced localized increase in the temperature of water (□); Janus vesicles before concentration in a magnetic field (∘); and Janus vesicles after concentration in a magnetic field (Δ).

FIG. 13B is a graph of the fluorescence intensity release profile from Janus vesicles before (∘) and after (Δ) being concentrated in a magnetic field upon laser irradiation and from Janus vesicles without laser irradiation (□).

FIG. 14A is a graph of the transverse relaxivity (r₂) of homogeneous vesicles containing 20 nm AuNPs. The corresponding r₂ is 110.6 s⁻¹.

FIG. 14B is a graph of the transverse relaxivity (r₂) of hemispherical Janus vesicles containing 20 nm AuNPs. The corresponding r₂ is 190.2 s⁻¹.

FIG. 15A is a SEM image of spherical Janus vesicles with 50 nm AuNPs before being irradiated by 655 nm continuous wave (CW) laser (0.35 W/cm²) for 4 minutes.

FIG. 15B is a SEM image of spherical Janus vesicles with 50 nm AuNPs after being irradiated by 655 nm continuous wave (CW) laser (0.35 W/cm²) for 4 minutes.

FIG. 16 is a graph showing the time-dependent fluorescence spectra of fluorescein isothiocyanate (FITC) released from the hemispherical Janus vesicles with laser irradiation by 655 nm continuation wave laser (0.35 W/cm²). Fluorescence intensity at 520 nm gradually increased with laser irradiation time.

FIG. 17A are in vivo 2D ultrasonic (US) images, photoacoustic (PA) images, and merged images of tumor tissues before and after the intratumoral injection of hemispherical Janus vesicles containing 50 nm or 20 nm AuNPs.

FIG. 17B are in vivo magnetic resonance (MR) images of tumor tissues before and after injection of hemispherical Janus vesicles containing 50 nm AuNPs and 15 nm MNPs. The mass of AuNPs and MNPs injected were 16 μg and 2.0 respectively.

FIG. 18 is a bar graph of the photoacoustic intensities of tumor tissues before and after intratumoral administration of hemispherical Janus vesicles containing 50 nm AuNPs or 20 nm AuNPs.

FIG. 19A are in vivo MR images of whole athymic nude mice bearing

U87MG tumors on the hind leg and corresponding tumor area (insets) before and after intravenous injection of hemispherical Janus vesicles containing 50 nm AuNPs and 15 nm MNPs, when a magnet is applied to the tumor. Arrows indicate a dark area in the tumor before and after the injection.

FIG. 19B are in vivo 2D ultrasonic (US), photoacoustic (PA), and merged images (left to right) of tumor tissues before and after intravenous injection of the hemispherical Janus vesicles with and without a magnet attached to the leg bearing tumors.

FIG. 20 is a bar graph of the photoacoustic intensities of tumor tissues before and two hours after intratumoral administration of the hemispherical Janus vesicles containing 50 nm AuNPs or 15 nm AuNPs with and without a magnet attached to the tumors.

FIG. 21 is a line graph of the temperature-dependent heating curves of a solution of Janus vesicles comprising 50 AuNPs ( [symbol] ), 20 nm AuNPs ( - - - ), MNP micelles ( ● ● ● ● ), and pure water ( - ● - ) after exposure to 808 nm near infrared laser at a power density of 0.5 W/cm² for 5 minutes. The temperature of solution of Janus vesicles with 50 nm AuNPs is higher than for the other solutions.

FIG. 22A is a line graph of the temperature-dependent heating curves of a solution of Janus vesicles comprising 50 AuNPs ( [symbol] ), 20 nm AuNPs ( - - - ), MNP micelles ( ● ● ● ● ), and pure water ( - ● - ) after exposure to 808 nm near infrared laser at a power density of 1.0 W/cm² for 5 minutes. The temperature of solution of Janus vesicles with 50 nm AuNPs is higher than for the other solutions.

FIG. 22B are thermal images of cuvettes containing (from top to bottom) a solution of Janus vesicles comprising 50 AuNPs, a solution of 20 nm AuNPs, a solution of MNP micelles, and pure water after exposure to 808 nm near infrared laser at a power density of 1.0 W/cm² for 5 minutes. The temperature of solution of Janus vesicles with 50 nm AuNPs is higher than for the other solutions.

FIG. 23 is schematic illustrating the fabrication of magnetic vesicles (MVs) with tunable wall thickness via cooperative assembly of BCP-grafted superparamagnetic iron oxide nanoparticles (SPIONs) and free PS-b-PAA.

FIG. 24 is a schematic illustrating the utilization of MVs for imaging-guided magnetic delivery of doxorubicin (Dox) into tumor-bearing mice.

FIG. 25A is a TEM image of SPIONs before the self-assembly. The scale bars represent 100 nm.

FIG. 25B is a TEM image of SPIONs before the self-assembly. The scale bars represent 20 nm.

FIG. 26A is a line graph of the size distribution of SPIONs as determined using TEM analysis.

FIG. 26B are line graphs of the dynamic light scattering analysis of the hydrodynamic diameter of SPIONs in THF before (SPIONs) and after (BCP-SPIONs) the grafting of amphiphilic PEO-b-PS on the surface.

FIG. 27 is a line graph of the thermogravimetric analysis (TGA) of PEO-b-PS-tethered SPIONs.

FIG. 28A is a STEM image of prepared multilayered magnetic vesicles (MuMVs).

FIG. 28B is a STEM image of prepared multilayered magnetic vesicles (MuMVs).

FIG. 28C is an energy-dispersive X-ray spectroscopy (EDS) image of Fe and in the MuMVs.

FIG. 29A is a SEM image of MuMVs self-assembled from BCP-SPIONs.

FIG. 29B is a TEM image of MuMVs self-assembled from BCP-SPIONs.

FIG. 30A is a TEM image of MuMVs self-assembled from BCP-SPIONs at a −60° tilt angle.

FIG. 30B is a TEM image of MuMVs self-assembled from BCP-SPIONs at a −30° tilt angle.

FIG. 30C is a TEM image of MuMVs self-assembled from BCP-SPIONs at a 30° tilt angle.

FIG. 30D is a TEM image of MuMVs self-assembled from BCP-SPIONs at a 60° tilt angle.

FIG. 31A is a STEM image for MuMVs showing the vesicular structure of the self-assembly. The image also includes a Fe intensity line scan.

FIG. 31B is a STEM image for monolayer magnetic vesicles (MoMVs) showing the vesicular structure of the self-assembly. The image also includes a Fe intensity line scan.

FIG. 32A is a line graph of the diameter of MuMVs dried on a TEM grid as determined using TEM analysis.

FIG. 32B is a line graph of the hydrodynamic diameter of MuMVs dispersed in water determined using dynamic light scattering.

FIG. 33A is a line graph of the Zeta potential measurement of MuMVs. The measurement indicates that the MuMVs are negatively charged due to the presence of carboxyl groups in PS-b-PAA.

FIG. 33B is a line graph of the hydrodynamic size distribution of MuMVs in phosphate buffered saline (PBS) and PBS supplemented with 10% fetal bovine serum (FBS).

FIG. 34A is a TEM image of MoMVs. The scale bars represent 200 nm.

FIG. 34B is a TEM image of double-layered magnetic vesicles (DoMVs). The scale bars represent 200 nm.

FIG. 35A is a TEM image of MuMVs. The scale bars represent 200 nm.

FIG. 35B is a TEM image of MuMVs. The scale bars represent 300 nm.

FIG. 36A is a scatter plot of the membrane thickness of MVs as a function of weight ratio of PS-b-PAA to BCP-SPIONs (W_{BCP}/W_{SPION}).

FIG. 36B is a scatter plot of the self-assembly of BCP-SPIONs with varying amounts of SPIONS and additional BCP of PS-b-PAA.

FIG. 37A is a SEM image of MoMVs. The occasional buckling and collapse of the membrane indicates the formation of hollow vesicular structures. The scale bar represent 500 nm.

FIG. 37B is a TEM image of MoMVs. The occasional buckling and collapse of the membrane indicates the formation of hollow vesicular structures. The scale bar represent 500 nm.

FIG. 38A is a SEM image of DoMVs. The wrinkling and buckling of the membrane indicates the formation of hollow vesicular structures. The scale bar represent 500 nm.

FIG. 38B is a TEM image of MoMVs. The wrinkling and buckling of the membrane indicates the formation of hollow vesicular structures. The scale bar represent 500 nm.

FIG. 39A is a SEM image of MuMVs. The wrinkling and buckling of the membrane indicates the formation of hollow vesicular structures. The scale bar represent 500 nm.

FIG. 39B is a TEM image of MuMVs. The wrinkling and buckling of the membrane indicates the formation of hollow vesicular structures. The scale bar represent 500 nm.

FIG. 40A is a TEM image of MuMVs. The scale bar represent 200 nm.

FIG. 40B is a TEM image of MuMVs. The scale bar represent 200 nm.

FIG. 40C is a TEM image of MuMVs. The scale bar represent 200 nm.

FIG. 40D is a TEM image of MuMVs. The scale bar represent 200 nm.

FIG. 41A is a SEM image of a magnetic aggregate. The scale bar represents 5 μm.

FIG. 41B is a SEM image of a magnetic aggregate. The scale bar represents 500 nm.

FIG. 42A is a TEM image of a magnetic aggregate. The scale bar represents 1 μm.

FIG. 42B is a TEM image of a magnetic aggregate. The scale bar represents 200 nm.

FIG. 43 is a schematic illustrating the mechanism for the formation of MoMVs, DoMVs, and MuMVs at different WB_{CP}/W_{SPION} ratios due to the cooperative interaction between BCP-grafted SPIONs and free PS-b-PAA.

FIG. 44A is a scatter plot of the hydrodynamic diameter of SPIONs as a function of the W_{BCP}/W_{SPION} ratio.

FIG. 44B is a scatter plot of the weight fraction of total BCPs in hybrid BCP-SPIONs as a function of increasing W_{BCP}/W_{SPION} ratio.

FIG. 45A is a SEM image of irregular aggregrates assembled from a mixture of PS-b-PEO grafted SPIONs and free PS-b-PEO. When PS-b-PAA was replaced by PS-b-PEO without affinity to the surface of SPIONs, the assembly did not produce MVs with tunable layers of SPIONs in the membrane. The scale bar represents 5 μm.

FIG. 45B is a SEM image of irregular aggregrates assembled from a mixture of PS-b-PEO grafted SPIONs and free PS-b-PEO. The scale bar represents 10 μm.

FIG. 46A is a SEM image of irregular aggregrates assembled from a mixture of PS-b-PEO grafted SPIONs and free PS-b-PEO. The scale bar represents 500 nm.

FIG. 46B is a SEM image of irregular aggregrates assembled from a mixture of PS-b-PEO grafted SPIONs and free PS-b-PEO. The scale bar represents 300 nm.

FIG. 47A is a hysteresis curve of MuMVs measured at 2 K and 300 K.

FIG. 47B are line graphs of the spin-spin 1/T₂ relaxation rates of different nanostructures as a function of iron concentration.

FIG. 48A is a bar graph of magnetization of each grain in individual SPIONs and MVs and the corresponding net magnetization of SPIONs and MVs.

FIG. 48B is a Mill image that has been T₂-weighted of different morphologies with various iron concentrations.

FIG. 49A is a hysteresis curve of SPIONs measured at 2 K and 300 K.

FIG. 49B is a hysteresis curve of MoMVs measured at 2 K and 300 K.

FIG. 49C is a hysteresis curve of DoMVs measured at 2 K and 300 K.

FIG. 50A is a hysteresis curve showing the magnetization of individual SPIONs obtained by fitting the data into the Langevin paramagnetic function.

FIG. 50B is a hysteresis curve showing the magnetization of individual SPIONs in MoMVs obtained by fitting the data into the Langevin paramagnetic function.

FIG. 51A is a hysteresis curve showing the magnetization of individual SPIONs in DoMVs obtained by fitting the data into the Langevin paramagnetic function.

FIG. 51B is a hysteresis curve showing the magnetization of individual SPIONs in MuMVs obtained by fitting the data into the Langevin paramagnetic function.

FIG. 52 are line graphs showing the spin-spin 1/T₂ relaxation rates of MuMVs before (MuMVs) and after (Dox-MuMVs) doxorubicin loading as a function of iron concentration.

FIG. 53A is a bar graph of the loading content of doxorubicin (Dox) in MuMVs as a function of the initial concentration of Dox.

FIG. 53B is a line graph of the loading content of doxorubicin (Dox) in MuMVs as a function of the initial concentration of Dox.

FIG. 54A is a SEM image of assemblies of BCP-SPIONs by film rehydration of building blocks in Dox solution with 1.5 mg/mL.

FIG. 54B is a SEM image of assemblies of BCP-SPIONs by film rehydration of building blocks in Dox solution with 2.0 mg/mL.

FIG. 55A are line graphs of the controlled in vitro release of Dox from MVs with different contents of PS-b-PAA added in the assembly: MV₀ (MoMVs, W_{BCP}=0); MV₁ (DoMVs, W_{BCP}=0.8); MV₂ (MuMVs, W_{BCP}=1.6); and MV₃ (MuMVs, W_{BCP}=3.2).

FIG. 55B are bar graphs of the in vitro cytotoxicity of Dox, Dox-MuMVs, RGD-Dox-MuMVs, and blank MuMVs to U87MG cells after incubation for 12 hours.

FIG. 56 are confocal microscope images showing enhanced targeting and Dox delivery from Dox-loaded FL-RGD-MuMVs to U87MG cells. The nuclei were stained with DAPI and the vesicular membranes were labelled with fluoresceinamine. Cells treated with PBS and Dox-load FL-MuMVs were used as control groups. Scale bars represent 20 μm.

FIG. 57A are line graphs of the controlled in vitro release of Dox from MVs with different membrane thickness fitting the linear form of the empirical Korsmeyer-Peppas equation. The formation conditions of the MVs are: MV₁ (DoMVs, W_{BCP}=0.8); MV₂ (MuMVs, W_{BCP}=1.6); and MV₃ (MuMVs, W_{BCP}=3.2).

FIG. 57B are line graphs of the reduced negative charge of MuMVs after conjugation with fluoresceinamine (FL-MuMVs) and RGD peptide (RGD-MuMVs).

FIG. 58 are TEM images of U87MG cells incubated with MuMVs for 1 hour. The arrows denote the vesicles inside the cell.

FIG. 59 are in vivo T₂-weighted MR images of tumor areas (shown in insets) in U87MG tumor-bearing mice pre-injection and 60 minutes after the intravenous injection of different sample groups: Dox-MVs (magnet ±) and RGD-Dox MVs (magnet ±).

FIG. 60A are in vivo fluorescent images of Dox in tumors (shown in insets) 1 hour after the intravenous injection of different sample groups: Dox-MVs (magnet ±) and RGD-Dox MVs (magnet ±).

FIG. 60B is a bar graph of the quantitative analysis of fluorescence intensity in corresponding tumor regions of different sample groups: Dox-MVs (magnet ±) and RGD-Dox MVs (magnet ±).

FIG. 61A is a line graph of tumor growth over time for tumor-bearing mice after different treatments: phosphate buffered saline (PBS), free doxorubicin (Dox), Dox-MuMVs (magnet ±), and RGD-Dox-MuMVs (magnet ±). Error bar represent the standard deviation of 5 mice per group.

FIG. 61B is a line graph of survival rate over time for tumor-bearing mice after different treatments: phosphate buffered saline (PBS), free doxorubicin (Dox), Dox-MuMVs (magnet ±), and RGD-Dox-MuMVs (magnet ±). Error bar represent the standard deviation of 5 mice per group.

FIG. 61C is a line graph of body weight over time for tumor-bearing mice after different treatments: phosphate buffered saline (PBS), free doxorubicin (Dox), Dox-MuMVs (magnet ±), and RGD-Dox-MuMVs (magnet ±). Error bar represent the standard deviation of 5 mice per group.

FIG. 62A are in vivo T₂-weighted MR images of the biodistribution of doxorubicin after intravenous injection of Dox-MuMVs (magnet −) and RGD-Dox-MuMVs (magnet +) into subcutaneous U87MG tumor-bearing mice.

FIG. 62B is a bar graph of the biodistribution of doxorubicin after intravenous injection of Dox-MuMVs (magnet −) and RGD-Dox-MuMVs (magnet +) into subcutaneous U87MG tumor-bearing mice.

FIG. 63A is a SEM image of MuMVs before filtration through a 200 nm filter. Scale bars represent 200 nm.

FIG. 63B is a SEM image of MuMVs after filtration through a 200 nm filter. Scale bars represent 200 nm.

### DETAILED DESCRIPTION OF THE INVENTION

Unless defined otherwise, all technical and scientific terms used herein have the same meaning as commonly understood by one of ordinary skill in the art to which the invention pertains. The following definitions supplement those in the art and are directed to the current application and are not to be imputed to any related or unrelated case, e.g., to any commonly owned patent or application. Although any methods and materials similar or equivalent to those described herein can be used in the practice for testing of the present invention, the preferred materials and methods are described herein. Accordingly, the terminology used herein is for the purpose of describing particular embodiments only, and is not intended to be limiting.

As used in this specification and the appended claims, the singular forms “a,” “an” and “the” include plural referents unless the context clearly dictates otherwise. Thus, for example, reference to “a nanostructure” includes a plurality of such nanostructures, and the like.

The term “about” as used herein indicates the value of a given quantity varies by ±10% of the value, or optionally ±5% of the value, or in some embodiments, by ±1% of the value so described. For example, “about 100 nm” encompasses a range of sizes from 90 nm to 110 nm, inclusive.

A “nanostructure” is a structure having at least one region or characteristic dimension with a dimension of less than about 500 nm. In some embodiments, the nanostructure has a dimension of less than about 200 nm, less than about 100 nm, less than about 50 nm, less than about 20 nm, or less than about 10 nm. Typically, the region or characteristic dimension will be along the smallest axis of the structure. Examples of such structures include nanowires, nanorods, nanotubes, branched nanostructures, nanotetrapods, nanotripods, nanobipods, nanocrystals, nanodots, quantum dots, nanoparticles, and the like. Nanostructures can be, e.g., substantially crystalline, substantially monocrystalline, polycrystalline, amorphous, or a combination thereof. In some embodiments, each of the three dimensions of the nanostructure has a dimension of less than about 500 nm, less than about 200 nm, less than about 100 nm, less than about 50 nm, less than about 20 nm, or less than about 10 nm. In some embodiments, the nanostructure is a nanoparticle.

As used herein, the “diameter” of a nanostructure refers to the diameter of a cross-section normal to a first axis of the nanostructure, where the first axis has the greatest difference in length with respect to the second and third axes (the second and third axes are the two axes whose lengths most nearly equal each other). The first axis is not necessarily the longest axis of the nanoparticle; e.g., for a disk-shaped nanostructure, the cross-section would be a substantially circular cross-section normal to the short longitudinal axis of the disk. Where the cross-section is not circular, the diameter is the average of the major and minor axes of that cross-section. For an elongated or high aspect ratio nanostructure, such as a nanowire, the diameter is measured across a cross-section perpendicular to the longest axis of the nanowire. For a spherical nanostructure, the diameter is measured from one side to the other through the center of the sphere.

As used herein, the “transverse relaxivity” or “transverse relaxation rate” (r₂) is a measurement of the increase of the water proton relaxation rate induced by 1 mmol per liter of paramagnetic center. The transverse relaxavity of a magnetic vesicle can be measured using the formula:

r₂=1/T₂

wherein T₂ is the transverse relaxation time measured using a magnetic resonance imaging spectrometer.

In some embodiments, the present disclosure provides a composition comprising:
(a) a first block copolymer comprising at least two polymer blocks, wherein at least one of the polymer blocks has been functionalized;
(b) a plurality of first inorganic nanoparticles bound to the surface of the first block copolymer;
(c) a second block copolymer comprising at least two polymer blocks; and
(d) a plurality of second inorganic nanoparticles; or
(a′) a first block copolymer comprising at least two polymer blocks, wherein at least one of the polymer blocks has been functionalized;
(b′) a plurality of small molecules bound to the surface of the first block copolymer;
(c′) a second block copolymer comprising at least two polymer blocks; and
(d′) a plurality of inorganic nanoparticles, wherein the plurality of small molecules are bound to the surface of the inorganic nanoparticles;
   wherein the composition is in the form of vesicles.

In some embodiments, the present disclosure provides a composition comprising:
(a) a first block copolymer comprising at least two polymer blocks, wherein at least one of the polymer blocks has been functionalized;
(b) a plurality of first inorganic nanoparticles bound to the surface of the first block copolymer;
(c) a second block copolymer comprising at least two polymer blocks; and
(d) a plurality of second inorganic nanoparticles;
   wherein the composition is in the form of vesicles.

In some embodiments, the present disclosure provides a composition comprising:
(a′) a first block copolymer comprising at least two polymer blocks, wherein at least one of the polymer blocks has been functionalized;
(b′) a plurality of small molecules bound to the surface of the first block copolymer;
(c′) a second block copolymer comprising at least two polymer blocks; and
(d′) a plurality of inorganic nanoparticles, wherein the plurality of small molecules are bound to the surface of the inorganic nanoparticles;
   wherein the composition is in the form of vesicles.

### Inorganic Nanoparticles

In some embodiments, the composition comprises a plurality of first inorganic nanoparticles. In some embodiments, the composition comprises a plurality of first inorganic nanoparticles and a plurality of second inorganic nanoparticles.

In some embodiments, the inorganic nanoparticles comprise an iron oxide. In some embodiments, the inorganic nanoparticles comprise an iron oxide such as Fe₂O₃, Fe₃O₄, or MFe₂O₄ (M=Fe, Co, or Mn). In some embodiments, the inorganic nanoparticles comprise Fe₃O₄.

In some embodiments, the inorganic nanoparticles comprise Au, Pt, Ag, Pd, Cu, or titanium oxide. In some embodiments, the inorganic nanoparticles comprise Au.

In some embodiments, the inorganic nanoparticles have a diameter between 10 nm and 100 nm. In some embodiments, the inorganic nanoparticles have a diameter between about 10 nm and about 100 nm, about 10 nm and about 80 nm, about 10 nm and about 60 nm, about 10 nm and about 50 nm, about 10 nm and about 40 nm, about 10 nm and about 30 nm, about 10 nm and about 25 nm, about 10 nm and about 20 nm, about 10 nm and about 15 nm, about 15 nm and about 100 nm, about 15 nm and about 80 nm, about 15 nm and about 60 nm, about 15 nm and about 50 nm, about 15 nm and about 40 nm, about 15 nm and about 30 nm, about 15 nm and about 25 nm, about 15 nm and about 20 nm, about 20 nm and about 100 nm, about 20 nm and about 80 nm, about 20 nm and about 60 nm, about 20 nm and about 50 nm, about 20 nm and about 40 nm, about 20 nm and about 30 nm, about 20 nm and about 25 nm, about 25 nm and about 100 nm, about 25 nm and about 80 nm, about 25 nm and about 60 nm, about 25 nm and about 50 nm, about 25 nm and about 40 nm, about 25 nm and about 30 nm, about 30 nm and about 100 nm, about 30 nm and about 80 nm, about 30 nm and about 60 nm, about 30 nm and about 50 nm, about 30 nm and about 40 nm, about 40 nm and about 100 nm, about 40 nm and about 80 nm, about 40 nm and about 60 nm, about 40 nm and about 50 nm, about 50 nm and about 120 nm, about 50 nm and about 80 nm, about 50 nm and about 60 nm, about 60 nm and about 100 nm, about 60 nm and about 80 nm, or about 80 nm and 100 nm.

In some embodiments, the inorganic nanoparticle comprises Au and has a diameter between about 20 nm and about 50 nm. In some embodiments, the inorganic nanoparticle comprises Au and has a diameter of about 20 nm, about 30 nm, or about 50 nm.

In some embodiments, the inorganic nanoparticle comprises Fe₃O₄ and has a diameter between about 15 nm and about 25 nm. In some embodiments, the inorganic nanoparticle comprises Fe₃O₄ and has a diameter of about 15 nm or about 25 nm.

In some embodiments, the composition comprises a plurality of first inorganic nanoparticles comprising Au.

In some embodiments, the composition comprises a plurality of first inorganic nanoparticles comprising Fe₃O₄ and a plurality of second inorganic nanoparticles comprising Au. In some embodiments, the composition comprises a plurality of first inorganic nanoparticles comprising Fe₃O₄ having a diameter between about 15 nm and about 25 nm and a plurality of second inorganic nanoparticles comprising Au having a diameter between about 20 nm and about 50 nm.

### Block Copolymer

As used herein, the term “polymer block” refers to a grouping of multiple monomer units of a single type (i.e., a homopolymer block) or multiple types (i.e., a copolymer block) of constitutional units into a continuous polymer chain.

As used herein, the term “block copolymer” refers to a polymer composed of chains where each chain contains two or more polymer blocks. A wide variety of block polymers are contemplated herein including diblock copolymers (i.e., polymers including two polymer blocks), triblock copolymers (i.e., polymers including three polymer blocks), multiblock copolymers (i.e., polymers including more than three polymer blocks), and combinations thereof

In some embodiments, the block copolymer comprises at least one block of poly(9,9-bis(6′-N,N,N-trimethylammonium)-hexyl)-fluorene phenylene) (PFP), polydimethylsiloxane (PDMS), poly(4-vinylpyridine) (P4VP), poly(-vinylpyridine) (P2VP), hydroxypropyl methylcellulose (HPMC), polyethylene glycol (PEG), poly(ethylene oxide)-co-poly(propylene oxide) di- or multiblock copolymers, poly(vinyl alcohol) (PVA), poly(ethylene-co-vinyl alcohol), poly(acrylic acid) (PAA), poly(ethyloxazoline), a poly(alkylacrylate), poly(acrylamide), a poly(N-alkylacrylamide), a poly(N,N-dialkylacrylamide), poly(propylene glycol) (PPG), poly(propylene oxide), partially or fully hydrolyzed poly(vinyl alcohol), dextran, polystyrene (PS), polyethylene (PE), polypropylene (PP), polychloroprene (CR), a polyvinyl ether, poly(vinyl acetate), poly(vinyl chloride) (PVC), poly(isoprene), poly(ethylene), poly(butadiene), a polysiloxane, a polyurethane (PU), a polyacrylate, or a polyacrylamide.

In some embodiments, the block copolymer comprises at least two polymer blocks (i.e., a first polymer block and a second polymer block) that are substantially immiscible in one another. In some embodiments, the block copolymer comprises a first polymer block and a second polymer block with a number average molecular weight ratio in a range of from about 5:95 to about 95:5, about 5:95 to about 90:10, about 5:95 to about 80:20, about 5:95 to about 70:30, about 5:95 to about 60:40, about 5:95 to about 50:50, about 5:95 to about 40:60, about 5:95 to about 30:70, about 5:95 to about 20:80, about 5:95 to about 10:90, about 10:90 to about 95:5, about 10:90 to about 90:10, about 10:90 to about 80:20, about 10:90 to about 70:30, about 10:90 to about 60:40, about 10:90 to about 50:50, about 10:90 to about 40:60, about 10:90 to about 30:70, about 10:90 to about 20:80, about 20:80 to about 95:5, about 20:80 to about 90:10, about 20:80 to about 80:20, about 20:80 to about 70:30, about 20:80 to about 60:40, about 20:80 to about 50:50, about 20:80 to about 40:60, about 20:80 to about 30:70, about 30:70 to about 95:5, about 30:70 to about 90:10, about 30:70 to about 80:20, about 30:70 to about 70:30, about 30:70 to about 60:40, about 30:70 to about 50:50, about 30:70 to about 40:60, about 40:60 to about 95:5, about 40:60 to about 90:10, about 40:60 to about 80:20, about 40:60 to about 70:30, about 40:60 to about 60:40, about 40:60 to about 50:50, about 50:50 to about 95:5, about 50:50 to about 90:10, about 50:50 to about 80:20, about 50:50 to about 70:30, about 50:50 to about 60:40, about 60:40 to about 95:5, about 60:40 to about 90:10, about 60:40 to about 80:20, about 60:40 to about 70:30, about 70:30 to about 95:5, about 70:30 to about 90:10, about 70:30 to about 80:20, about 80:20 to about 95:5, about 80:20 to about 90:10, or about 90:10 to about 95:5.

In some embodiments, the polymer block is a functionalized polymer block. A functionalized polymer block contains an organic functional group such as an amine, quaternary ammonium, hydroxyl, thiol, carboxylate, carboxylic acid, sulfate, sulfonate, sulfonic acid, epoxide, phosphate, or phosphonate. In some embodiments, the polymer block is a functionalized polymer block that is functionalized with a thiol.

In some embodiments, the block copolymer is polystyrene-block-poly(4-vinylpyridine), polystyrene-block-poly(2-vinylpyridine), polyisoprene-b-poly(4-vinylpyridine), polybutadiene-block-poly(4-vinylpyridine), polyethylene-block-poly(4-vinylpyridine), polystyrene-block-poly(2-vinylpyridine), polyisoprene-b-poly(2-vinylpyridine), polybutadiene-block-poly(2-vinylpyridine), polyethylene-block-poly(2-vinylpyridine), polystyrene-block-poly(ethylene oxide) (PS-b-PEO), or polystyrene-block-poly(acrylic acid) (PS-b-PAA).

In some embodiments, the block copolymer is polystyrene-block-poly(ethylene oxide). In some embodiments, the number average molecular weight (kg/mol) of polystyrene to poly(ethylene oxide) is a number average molecular weight ratio in a range of from about 5:95 to about 95:5, about 5:95 to about 90:10, about 5:95 to about 80:20, about 5:95 to about 70:30, about 5:95 to about 60:40, about 5:95 to about 50:50, about 5:95 to about 40:60, about 5:95 to about 30:70, about 5:95 to about 20:80, about 5:95 to about 10:90, about 10:90 to about 95:5, about 10:90 to about 90:10, about 10:90 to about 80:20, about 10:90 to about 70:30, about 10:90 to about 60:40, about 10:90 to about 50:50, about 10:90 to about 40:60, about 10:90 to about 30:70, about 10:90 to about 20:80, about 20:80 to about 95:5, about 20:80 to about 90:10, about 20:80 to about 80:20, about 20:80 to about 70:30, about 20:80 to about 60:40, about 20:80 to about 50:50, about 20:80 to about 40:60, about 20:80 to about 30:70, about 30:70 to about 95:5, about 30:70 to about 90:10, about 30:70 to about 80:20, about 30:70 to about 70:30, about 30:70 to about 60:40, about 30:70 to about 50:50, about 30:70 to about 40:60, about 40:60 to about 95:5, about 40:60 to about 90:10, about 40:60 to about 80:20, about 40:60 to about 70:30, about 40:60 to about 60:40, about 40:60 to about 50:50, about 50:50 to about 95:5, about 50:50 to about 90:10, about 50:50 to about 80:20, about 50:50 to about 70:30, about 50:50 to about 60:40, about 60:40 to about 95:5, about 60:40 to about 90:10, about 60:40 to about 80:20, about 60:40 to about 70:30, about 70:30 to about 95:5, about 70:30 to about 90:10, about 70:30 to about 80:20, about 80:20 to about 95:5, about 80:20 to about 90:10, or about 90:10 to about 95:5. In some embodiments, the number average molecular weight (kg/mol) of polystyrene to poly(ethylene oxide) is 25:1. In some embodiments, polystyrene-block-poly(ethylene oxide) is PS₄₉₀-b-PEO₄₅.

In some embodiments, the block copolymer is polystyrene-block-poly(acrylic acid). In some embodiments the number average molecular weight (kg/mol) of polystyrene to poly(acrylic acid) is a number average molecular weight ratio in a range of from about 5:95 to about 95:5, about 5:95 to about 90:10, about 5:95 to about 80:20, about 5:95 to about 70:30, about 5:95 to about 60:40, about 5:95 to about 50:50, about 5:95 to about 40:60, about 5:95 to about 30:70, about 5:95 to about 20:80, about 5:95 to about 10:90, about 10:90 to about 95:5, about 10:90 to about 90:10, about 10:90 to about 80:20, about 10:90 to about 70:30, about 10:90 to about 60:40, about 10:90 to about 50:50, about 10:90 to about 40:60, about 10:90 to about 30:70, about 10:90 to about 20:80, about 20:80 to about 95:5, about 20:80 to about 90:10, about 20:80 to about 80:20, about 20:80 to about 70:30, about 20:80 to about 60:40, about 20:80 to about 50:50, about 20:80 to about 40:60, about 20:80 to about 30:70, about 30:70 to about 95:5, about 30:70 to about 90:10, about 30:70 to about 80:20, about 30:70 to about 70:30, about 30:70 to about 60:40, about 30:70 to about 50:50, about 30:70 to about 40:60, about 40:60 to about 95:5, about 40:60 to about 90:10, about 40:60 to about 80:20, about 40:60 to about 70:30, about 40:60 to about 60:40, about 40:60 to about 50:50, about 50:50 to about 95:5, about 50:50 to about 90:10, about 50:50 to about 80:20, about 50:50 to about 70:30, about 50:50 to about 60:40, about 60:40 to about 95:5, about 60:40 to about 90:10, about 60:40 to about 80:20, about 60:40 to about 70:30, about 70:30 to about 95:5, about 70:30 to about 90:10, about 70:30 to about 80:20, about 80:20 to about 95:5, about 80:20 to about 90:10, or about 90:10 to about 95:5. In some embodiments the number average molecular weight (kg/mol) of polystyrene to poly(acrylic acid) is 40:1. In some embodiments, the polystyrene-block-poly(acrylic acid) is PS₁₀₇-b-PAA₄.

### Functionalization of the Block Copolymer

To allow for the functionalization of a block copolymer, the block copolymer can be reacted with a functionalizing agent.

The term “functionalizing agent” as used herein refers to a chemical reagent that is used to modify the chemical composition of a polymer such that a desired functional group is covalently linked to the polymer at the end of the reaction. In some embodiments, the functionalizing agent is an alkylating agent, a cross-linking agent, a carboxylating agent, an oxidizing agent, a reducing agent, or an epoxidating agent. In some embodiments, the functionalizing agent is an alkyl halide, an aryl halide, an alkyl dihalide, an alkyl dialdehyde, or an alkyl diamine. In some embodiments, the functionalizing agent is glutaraldehyde, formic acid, chromic acid, sodium borohydride, sodium, 1,2-propylene oxide, glycidol, succinic anhydride, or succinimide.

Quaternary ammonium cations are positively charged polyatomic ions of the structure NR₄⁺, R being an alkyl group or an aryl group. Quaternary ammonium compounds are prepared by alkylation of tertiary amines, in a process called quaternization.

In some embodiments, at least one of polymer blocks is quaternized by exposing the polymer block to an alkylating agent.

The term “alkylating agent” as used herein refers to a reagent capable of placing an alkyl group onto a nucleophilic site. In some embodiments, the alkylating agent is an organic halide, an organic dihalide, an alkyl sulfate, an alkyl disulfate, or an alkyl or aryl disulfonate. In some embodiments, the alkylating agent is an organic dihalide, e.g., an alkyl dihalide, such as1,4-dibromobutane, 1,5-dibromopentane, 1,6-dibromohexane, 1,7-dibromoheptane, 1,8-dibromooctane, 1,9-dibromononane, 1,4-dichlorobutane, 1,5-dichloropentane, 1,6-dichlorohexane, 1,7-dichloroheptane, 1,8-dichlorooctane, 1,9-dichlorononane, and combinations thereof In some embodiment, the alkylating agent is an aryl disulfonate, such as anthraquinone-2,6-disulfonate or 1,5-naphthalene disulfonate. In some embodiments, the alkylating agent is benzyl bromide or benzyl chloride. In some embodiments, the alkylating agent is 1,4-dibromobutane.

In some embodiments, the block copolymer is admixed with a chain transfer agent before exposure to a functionalizing agent. In some embodiments, the chain transfer agent is 4-cyano-4-(phenylcarbonothioylthio)pentanoic acid (CPPA).

In some embodiments, the functionalizing agent is an amine. In some embodiments, the functionalizing agent is an amine selected from the group consisting of methylamine, dimethylamine, trimethylamine, ethylamine, aniline, n-butylamine, or butylamine.

In some embodiments, the chain transfer agent CPPA is admixed with poly(ethylene oxide) and polystyrene. The reaction produces polystyrene-b-poly(ethylene oxide) bearing a CPPA moiety at the PEO chain end. Reaction of polystyrene-b-poly(ethylene oxide) bearing a CPPA moiety at the PEO chain end with an amine provides polystyrene-b-poly(ethylene oxide) bearing a thiol group at the PEO chain end.

### Small Molecules

In some embodiments, the composition comprises a small molecule. In some embodiments, the composition comprises a small molecule bound to the surface of a block copolymer.

The percentage of small molecules bound to the surface of the functionalized block copolymer can be measured by ¹H NMR. In some embodiments, the mole percentage of small molecules bound to the surface of the functionalized block copolymer is between about 20% and about 100%, about 20% and about 80%, about 20% and about 60%, about 20% and about 40%, about 25% and about 100%, about 25% and about 80%, about 25% and about 60%, about 25% and about 40%, about 30% and about 100%, about 30% and about 80%, about 30% and about 60%, about 30% and about 40%, about 40% and about 100%, about 40% and about 80%, about 40% and about 60%, about 60% and about 100%, about 60% and about 80%, or about 80% and about 100%.

The percentage of small molecules bound to the surface of a block copolymer can be measured by ¹H NMR, wherein the bound small molecules are calculated using: (bound small molecules)/(bound +free small molecules).

In some embodiments, the small molecule is a neurotransmitter. In some embodiments, the small molecule is a neurotransmitter selected from the group consisting of glycine, glutamic acid, y-aminobutyric acid (GABA), glycine, dopamine, norepinephrine, epinephrine, serotonin, histamine, adenosine, adenosine triphosphate (ATP), and acetylcholine. In some embodiments, the small molecule is a neurotransmitter selected from the group consisting of dopamine, norepinephrine, epinephrine, serotonin, and histamine. In some embodiments, the small molecule is dopamine.

### Modification of Functionalized Copolymers

In some embodiments, inorganic nanoparticles can bind to the surface of a functionalized block copolymer.

The percentage of inorganic nanoparticles bound to the surface of the functionalized block copolymer can be measured by ¹H NMR. In some embodiments, the mole percentage of inorganic nanoparticles bound to the surface of the functionalized block copolymer is between about 20% and about 100%, about 20% and about 80%, about 20% and about 60%, about 20% and about 40%, about 25% and about 100%, about 25% and about 80%, about 25% and about 60%, about 25% and about 40%, about 30% and about 100%, about 30% and about 80%, about 30% and about 60%, about 30% and about 40%, about 40% and about 100%, about 40% and about 80%, about 40% and about 60%, about 60% and about 100%, about 60% and about 80%, or about 80% and about 100%.

The percentage of inorganic nanoparticles bound to the surface of a block copolymer can be measured by ¹H NMR, wherein the bound inorganic nanoparticles are calculated using: (bound inorganic nanoparticles)/(bound +free inorganic nanoparticles).

In some embodiments, the inorganic nanoparticle is Au and the copolymer is thiol-terminated polystyrene-b-poly(ethylene oxide). In some embodiments, the the mole percentage of Au bound to the thiol-terminated polystyrene-b-poly(ethylene oxide) is between about 20% and about 100% is between about 20% and about 100%, about 20% and about 80%, about 20% and about 60%, about 20% and about 40%, about 25% and about 100%, about 25% and about 80%, about 25% and about 60%, about 25% and about 40%, about 30% and about 100%, about 30% and about 80%, about 30% and about 60%, about 30% and about 40%, about 40% and about 100%, about 40% and about 80%, about 40% and about 60%, about 60% and about 100%, about 60% and about 80%, or about 80% and about 100%.

In some embodiments, the inorganic nanoparticle is Fe₃O₄ and the copolymer is thiol-terminated polystyrene-b-poly(ethylene oxide). In some embodiments, the mole percentage of Fe₃O₄ bound to the thiol-terminated polystyrene-b-poly(ethylene oxide) is between about 20% and about 100% is between about 20% and about 100%, about 20% and about 80%, about 20% and about 60%, about 20% and about 40%, about 25% and about 100%, about 25% and about 80%, about 25% and about 60%, about 25% and about 40%, about 30% and about 100%, about 30% and about 80%, about 30% and about 60%, about 30% and about 40%, about 40% and about 100%, about 40% and about 80%, about 40% and about 60%, about 60% and about 100%, about 60% and about 80%, or about 80% and about 100%.

### Magnetic Vesicle

The first and second block copolymers are characterized by their ability to self-assemble into a magnetic vesicle. Self-assembly occurs in the presence of a solvent and, although not required, may occur in the presence of water or other aqueous containing solution. In some embodiments, the solvent is ethanol, hexane, pentane, toluene, benzene, diethylether, acetone, ethyl acetate, dichloromethane (methylene chloride), chloroform, tetrahydrofuran, dimethylformamide, or N-methylpyrrolidinone. In some embodiments, the solvent is tetrahydrofuran.

The magnetic vesicle can include other components which do not interfere with its ability to self-assemble into a vesicle and do not alter its biocompatible, and/or biodegradable properties. Such components can be included to enhance some property of the vesicle such as its size, permeation properties, hydrophobicity, hydrophilicity, and/or charge or alternatively to enable delivery of the vesicle to a specific desired target within the animal. As an example, the surface of the vesicle may be modified by the addition of ligands specific for receptors of a cell or tissue type to which delivery of the agent is desired. As an example, antibodies for a cancer antigen so attached may be used to direct the vesicles to a cancer cell expressing the antigen. Other non-limiting examples of ligands suitable for targeting vesicles to specific cell types include carbohydrates, proteins, folic acid, peptides, and permeation enhancers. In some embodiments, the magnetic vesicles further comprises a therapeutic agent.

In self-assembling into magnetic vesicles, the block copolymer molecules form closed polymer shells generally hemispherical or spherical in nature. The closed polymer shells can shield an encapsulated therapeutic agent for delivery from conditions which might degrade or inactivate the agent if delivered in the absence of the vesicle. As an example, a magnetic vesicle of the disclosure would allow for oral delivery of agents such as small peptides, which would otherwise likely be enzymatically degraded prior to sorption by the body.

The term “magnetic vesicle” is intended to refer to spontaneously forming nanoscale structures containing at least two block copolymer and at least one inorganic nanoparticle bound to at least one block copolymer. Magnetic vesicles of the invention are generally hemispherical or spherical in shape with an internal, hollow void. Upon self-assembly a magnetic vesicle is stabilized for delivery. Because of the vesicle's inherent stability, the vesicle does not require, and is preferably is not subjected to, induced crosslinking once the vesicle is formed. Rather, a magnetic vesicle of the present invention is stabilized through the strength of hydrophobic interactions between the hydrophobic segments of such copolymers and through the strong segregation between the hydrophilic and hydrophobic fragments. Additional stabilization can be gained by specific interactions such as crystallization and electrostatic interactions. The identity of the polymer blocks of the present invention are chosen such that the hydrophilic and hydrophobic properties of the polymer blocks impart stability sufficient to encapsulate an agent for the delivery to the desired cells within an animal.

Regardless of the conditions of self-assembly, vesicles of various sizes can be obtained. In some embodiments, the magnetic vesicles have a diameter between about 10 nm and about 1000 nm, about 10 nm and about 800 nm, about 10 nm and about 600 nm, about 10 nm and about 400 nm, about 10 nm and about 200 nm, about 10 nm and about 100 nm, about 10 nm and about 50 nm, about 50 nm and about 1000 nm, about 50 nm and about 800 nm, about 50 nm and about 600 nm, about 50 nm and about 400 nm, about 50 nm and about 200 nm, about 50 nm and about 100 nm, about 100 nm and about 1000 nm, about 100 nm and about 800 nm, about 100 nm and about 600 nm, about 100 nm and about 400 nm, about 100 nm and about 200 nm, about 200 nm and about 1000 nm, about 200 nm and about 800 nm, about 200 nm and about 600 nm, about 200 nm and about 400 nm, about 400 nm and about 1000 nm, about 400 nm and about 800 nm, about 400 nm and about 600 nm, about 600 nm and about 1000 nm, about 600 nm and about 800 nm, or about 800 nm and about 1000 nm.

In some embodiments, the transverse relaxivity (r₂) of the vesicle is between about 100 mM⁻¹s⁻¹ to about 600 mM⁻¹s⁻¹. In some embodiments, the transverse relaxivity of the vesicle is between about 100 mM⁻¹s⁻¹ to about 600 mM⁻¹s⁻¹, about 100 mM⁻¹s⁻¹ to about 500 mM⁻¹s⁻¹, about 100 mM⁻¹s⁻¹ to about 400 mM⁻¹s⁻¹, about 100 mM⁻¹s⁻¹ to about 300 mM⁻¹s⁻¹, about 100 mM⁻¹s⁻¹ to about 200 mM⁻¹s⁻¹, about 100 mM⁻¹s⁻¹ to about 150 mM⁻¹s⁻¹, about 150 mM⁻¹s⁻¹ to about 600 mM⁻¹s⁻¹, about 150 mM⁻¹s⁻¹ to about 500 mM⁻¹s⁻¹, about 150 mM⁻¹s⁻¹ to about 400 mM⁻¹s⁻¹, about 150 mM⁻¹s⁻¹ to about 300 mM⁻¹s⁻¹, about 200 mM⁻¹s⁻¹ to about 600 mM⁻¹s⁻¹, about 200 mM⁻¹s⁻¹ to about 500 mM⁻¹s⁻¹, about 200 mM⁻¹s⁻¹ to about 400 mM⁻¹s⁻¹, about 200 mM⁻¹s⁻¹ to about 300 mM⁻¹s⁻¹, about 300 mM⁻¹s⁻¹ to about 600 mM⁻¹s⁻¹, about 300 mM⁻¹s⁻¹ to about 500 mM⁻¹s⁻¹, about 300 mM⁻¹s⁻¹ to about 400 mM⁻¹s⁻¹, about 400 mM⁻¹s⁻¹ to about 600 mM⁻¹s⁻¹, about 400 mM⁻¹s⁻¹ to about 500 mM⁻¹s⁻¹, or about 500 mM⁻¹s⁻¹ to about 600 mM⁻¹s⁻¹. In some embodiments, the transverse relaxivity (r₂) of the vesicle is between about 150 mM⁻¹s⁻¹ to about 300 mM⁻¹s⁻¹.

### Therapeutic Agents for Encapsulation in a Magnetic Vesicle

The magnetic vesicles described herein are suitable for encapsulating a wide variety of agents, including but not limited to therapeutic, prophylactic, and diagnostic agents. The molecular size of an agent is generally not limiting, as both large and small molecular weight agents may be encapsulated. If necessary, larger vesicles may be used to accommodate larger molecules as agents and smaller vesicles may be used to accommodate smaller molecules as agents. Although both generally hydrophilic and generally hydrophobic agents may be encapsulated and delivered using such vesicles, it is a requirement that an agent be at least partially soluble in water. Non-limiting examples of therapeutic agents include proteins, polypeptides, peptides, nucleic acids, and synthetic organic molecules, or a mimetic of any one of the same. A nucleic acid may be a single-stranded or double-stranded DNA or RNA molecule and may further comprise an oligonucleotide. The nucleic acid may further comprise a vector such as a plasmid. Additionally, an agent may be modified prior to encapsulation, such as by glycosylation in the case of a protein, polypeptide, or peptide, or by the incorporation of analogues or labels for a nucleic acid. Therapeutic agents may function as hormones, vaccines, antibodies, antibiotics, chemotherapeutics, antisense, antiangiogenic agents, small interfering RNAs (siRNAs), or other function. Non-limiting examples of diagnostic agents include metal particles, radiolabels, and magnetic particles. In some embodiments, the therapeutic agent is a chemotherapeutic agent. Examples of chemotherapeutic agents include VEGF and VEGFR inhibitors such as bevacizumab (AVASTIN®), lapatinib (TYKERB®), axitinib (INLYTA®), sunitinib malate (SUTENT®), sorafenib (NEXAVAR®), and pazopanib (VOTRIENT®); aromatase inhibitors including steroids, such as atamestane, exemestane, and formestane, and non-steroids, such as aminoglutethimide, roglethimide, pyridoglutethimide, trilostane, testolactone, ketokonazole, vorozole, fadrozole, anastrozole, and letrozole; topoisomerase I inhibitors including topotecan, gimatecan, irinotecan, camptothecin and its analogues, 9-nitrocamptothecin, and the macromolecular camptothecin conjugate PNU-166148; topoisomerase II inhibitors including anthracyclines such as doxorubicin, daunorubicin, epirubicin, idarubicin, and nemorubicin; anthraquinones, such as mitoxantrone and losoxantrone; podophillotoxines, such as etoposide and teniposide; microtubulin polymerization inhibitors including taxanes, such as paclitaxel and docetaxel; vinca alkaloids, such as vinblastine, vinblastine sulfate, vincristine, and vincristine sulfate, and vinorelbine; discodermolides; cochicine and epothilones and derivatives thereof; alkylating agents including cyclophosphamide, ifosfamide, melphalan; nitrosoureas such as carmustine and lomustine; matrix metalloproteinase inhibitors (“MMP inhibitors”) include; collagen peptidomimetic and nonpeptidomimetic inhibitors, tetracycline derivatives, batimastat, marimastat, prinomastat, metastat, BMS-279251, BAY 12-9566, TAA211, MMI270B, and AAJ996; antimetabolites including 5-fluorouracil (5-FU), capecitabine, gemcitabine; DNA demethylating compounds, such as 5-azacytidine and decitabine; methotrexate and edatrexate; folic acid antagonists, such as pemetrexed; and platin compounds including carboplatin, cis-platin, cisplatinum, and oxaliplatin. In one embodiment, the therapeutic agent is doxorubicin.

The magnetic vesicles containing encapsulated agents may be packaged in dosage forms. Magnetic vesicles containing encapsulated agents may be packaged alone in such form or in combination with other active agents. Magnetic vesicles may further be packaged with an inert carrier that allows delivery of the vesicles as a tablet, capsule, or implant. For example, for oral delivery, the vesicle can be packaged in gastro-resistant pills which would allow the vesicle to bypass the acidic environment of the stomach. The number of vesicles for a particular dose may vary, depending on the amount of agent encapsulated by the vesicle. Higher or lower dosages may be attained in such form by increasing or decreasing, respectively, the number of magnetic vesicles comprising encapsulated agents or by increasing or decreasing the amount of agent encapsulated within each vesicle during assembly. In lieu of magnetic vesicles containing encapsulated agents, a mixture of an agent and triblock copolymer of the present invention may be packaged and delivered in dosage unit forms in the same manner as stated above.

### Method for Making Vesicle Compositions for Delivery of a Therapeutic Agent

Also provided herein is a method for making a vesicle composition for delivery of an agent. This method comprises first providing a first and a second block copolymer, wherein the first block copolymer is characterized as biocompatible, hydrophilic, and enzymatically degradable, wherein the second block copolymer is characterized as biodegradable and hydrophobic, and further wherein the first block copolymer and second block copolymer are characterized by the ability to self-assemble into a magnetic vesicle. The method thereafter comprises contacting the composition comprising:
(a) a first block copolymer comprising at least two polymer blocks, wherein at least one of the polymer blocks has been functionalized;
(b) a plurality of first inorganic nanoparticles bound to the surface of the first block copolymer;
(c) a second block copolymer comprising at least two polymer blocks; and
(d) a plurality of second inorganic nanoparticles; or
(a′) a first block copolymer comprising at least two polymer blocks, wherein at least one of the polymer blocks has been functionalized;
(b′) a plurality of small molecules bound to the surface of the first block copolymer;
(c′) a second block copolymer comprising at least two polymer blocks; and
(d′) a plurality of inorganic nanoparticles, wherein the plurality of small molecules are bound to the surface of the inorganic nanoparticles;
   wherein the composition is in the form of vesicles;
with an aqueous solution containing the therapeutic agent to be delivered, forming magnetic vesicles comprising the therapeutic agent encapsulated in the vesicle, thereby forming a composition in the form of vesicles for the delivery of the agent.

### Method for Administering a Therapeutic Agent to an Animal

Also provided herein is a method for using a composition of the present invention for administering an agent to an animal. This method includes first providing a composition comprising:
(a) a first block copolymer comprising at least two polymer blocks, wherein at least one of the polymer blocks has been functionalized;
(b) a plurality of first inorganic nanoparticles bound to the surface of the first block copolymer;
(c) a second block copolymer comprising at least two polymer blocks; and
(d) a plurality of second inorganic nanoparticles; or
(a′) a first block copolymer comprising at least two polymer blocks, wherein at least one of the polymer blocks has been functionalized;
(b′) a plurality of small molecules bound to the surface of the first block copolymer;
(c′) a second block copolymer comprising at least two polymer blocks; and
(d′) a plurality of inorganic nanoparticles, wherein the plurality of small molecules are bound to the surface of the inorganic nanoparticles;
   wherein the composition is in the form of vesicles;
and a therapeutic agent whose delivery to an animal is desired. The first block copolymer is characterized as biocompatible, hydrophilic, and enzymatically degradable, wherein the second block copolymer is characterized as biodegradable and hydrophobic, and further wherein the first block copolymer and second block copolymer are characterized by the ability to self-assemble into a magnetic vesicle. The therapeutic agent is characterized by the ability to be encapsulated in the self-assembled magnetic vesicle. The animal may be either non-human or human.

The magnetic vesicles containing encapsulated agents can be administered in dosage units. Magnetic vesicles containing encapsulated therapeutic agents can be administered alone in such form or in combination with other active agents. Magnetic vesicles can further be administered with an inert carrier that allows delivery of the vesicles as a tablet, capsule, or implant. The number of vesicles for a particular dose may vary, depending on the amount of therapeutic agent encapsulated by the vesicle. Higher or lower dosages may be attained in such form by increasing or decreasing, respectively, the number of magnetic vesicles comprising encapsulated therapeutic agents or by increasing or decreasing the amount of therapeutic agent encapsulated within each vesicle during assembly.

### Method for Making Vesicle Compositions for Imaging a Biological Target

Also provided herein is a method for making a vesicle composition for imaging a biological target. This method comprises first providing a first and a second block copolymer, wherein the first block copolymer is characterized as biocompatible, hydrophilic, and enzymatically degradable, wherein the second block copolymer is characterized as biodegradable and hydrophobic, and further wherein the first block copolymer and second block copolymer are characterized by the ability to self-assemble into a magnetic vesicle. The method comprises preparing a composition for imaging a biological target comprising:

(i) providing a composition comprising:
(a) a first block copolymer comprising at least two polymer blocks, wherein at least one of the polymer blocks has been functionalized;
(b) a plurality of first inorganic nanoparticles bound to the surface of the first block copolymer;
(c) a second block copolymer comprising at least two polymer blocks; and
(d) a plurality of second inorganic nanoparticles; or
(a′) a first block copolymer comprising at least two polymer blocks, wherein at least one of the polymer blocks has been functionalized;
(b′) a plurality of small molecules bound to the surface of the first block copolymer;
(c′) a second block copolymer comprising at least two polymer blocks; and
(d′) a plurality of inorganic nanoparticles, wherein the plurality of small molecules are bound to the surface of the inorganic nanoparticles;

wherein the composition is in the form of vesicles; and

(ii) detecting the vesicles.

In some embodiments, detecting the vesicles uses one or more of a fluorescence microscope, laser-confocal microscopy, cross-polarization microscopy, nuclear scintigraphy, positron emission tomography, single photon emission computed tomography, magnetic resonance imaging, photoacoustic imaging, magnetic resonance spectroscopy, computed tomography, or a combination thereof. In some embodiments, detecting the vesicles uses photoacoustic imaging. In some embodiments, detecting the vesicles uses magnetic resonance spectroscopy.

In some embodiments, the composition further comprises a fluorescent label.

As used herein, the term “fluorescent label” includes, but is not limited to, fluorescent imaging agents and fluorophores, that are chemical compounds, which when excited by exposure to a particular wavelength of light, emit light at a different wavelength. Fluorophores may be described in terms of their emission profile, or color, and are the component of a molecule that causes the molecule to be fluorescent. It is typically a functional group that absorbs energy of a specific wavelength or range of wavelengths and re-emit energy at different but equally specific wavelengths or ranges. In some embodiments, the fluorescent label is fluorosceinamine.

In some embodiments, the biological target is a nucleic acid, a protein, or a peptide. In some embodiments, the biological target is a nucleic acid material such as RNA, DNA, or a RNA/DNA hybrid. When the biological target material is a nucleic acid, it is preferably DNA, or RNA including but not limited to plasmid DNA, DNA fragments produced from restriction enzyme digestion, amplified DNA produced by an amplification reaction such as the polymerase chain reaction (PCR), single-stranded DNA, mRNA, or total RNA. In some embodiments, the biological target is an arginylglycylaspartic acid (RGD) peptide.

### EXAMPLES

The following examples are illustrative and non-limiting of the nanoparticle arrays, methods of making, and methods of using described herein. Suitable modifications and adaptations of the variety of conditions, formulations and other parameters normally encountered in the field and which are obvious to those skilled in the art in view of this disclosure are within the spirit and scope of the invention.

### Example 1

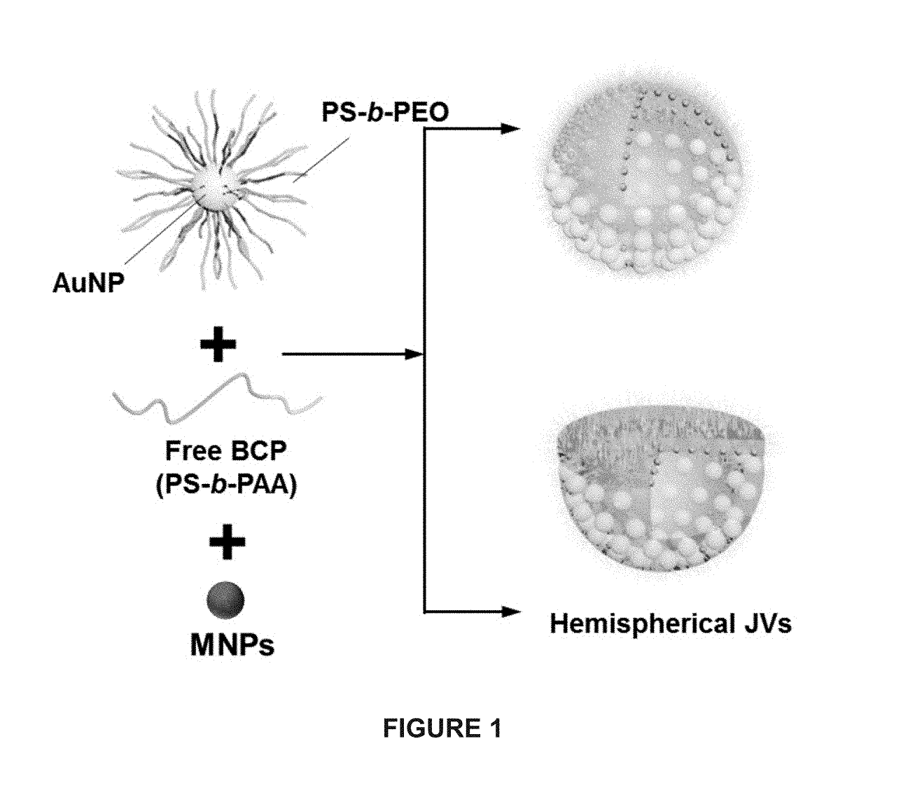

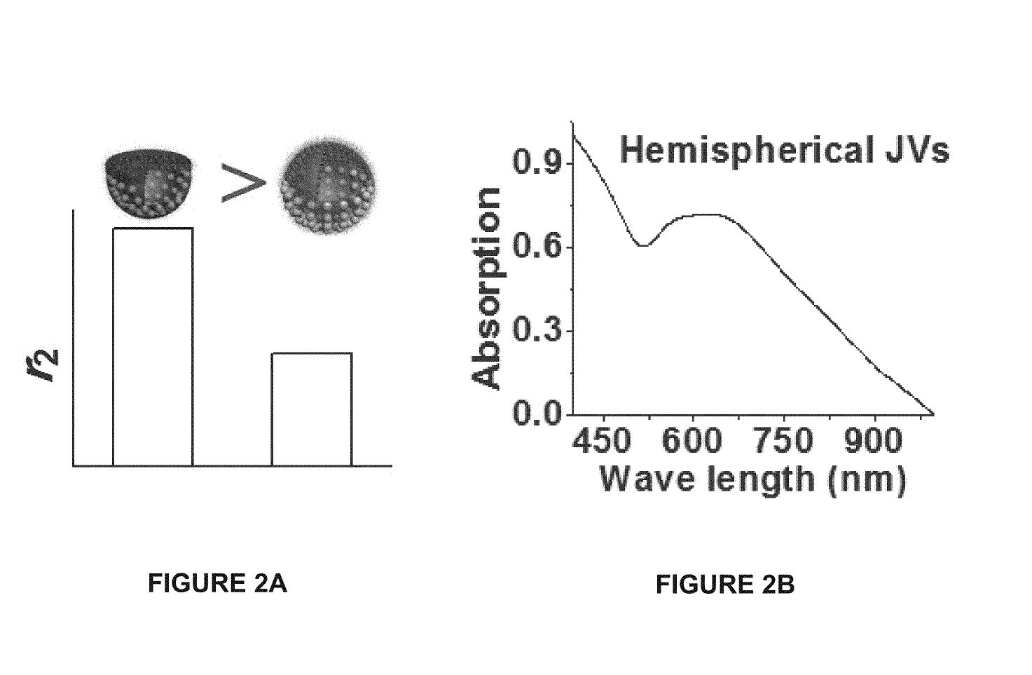

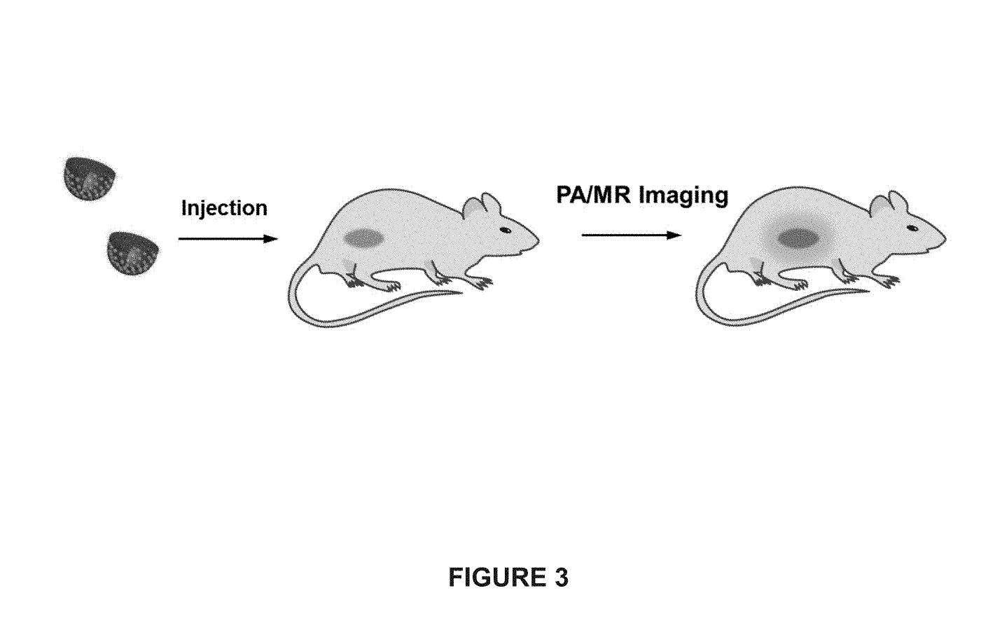

Magneto-plasmonic Janus vesicles (JVs) were fabricated by co-assembling a mixture of hydrophobic Fe₃O₄ magnetic nanoparticles (MNPs), an amphiphilic block copolymer (BCP) of polystyrene-b-poly(acrylic acid) (PS-b-PAA), and gold nanoparticles (AuNPs) grafted with polystyrene-b-poly (ethylene oxide) (PS-b-PEO) on the surface (FIG. 1). Depending on the size and mass fraction of nanoparticles (NPs) in the mixture, the assembly process produced spherical JVs (FIG. 1) and hemispherical JVs (FIG. 1). The hemispherical JVs containing 50 nm AuNPs and 15 nm MNPs exhibit a higher transverse relaxivity (r₂) value than both individual MNPs and spherical JVs, as a result of magnetic interactions between the MNPs within individual assemblies (FIG. 2A). Moreover, they show a strong absorption in the near infrared (NIR) range, due to the plasmonic coupling between neighboring AuNPs densely-packed within one half of the vesicular membrane. Model drugs can be encapsulated in the JVs and the release of payload can be triggered by NIR laser irradation. Furthermore, with external magnetic field, the JVs can be enriched in the tumor upon intravenous injection, leading to ˜2-3 times of signal enhancement in photoacoustic (PA) and magnetic resonance (MR) imaging of cancers, compared with that of control groups without an external magnetic field.

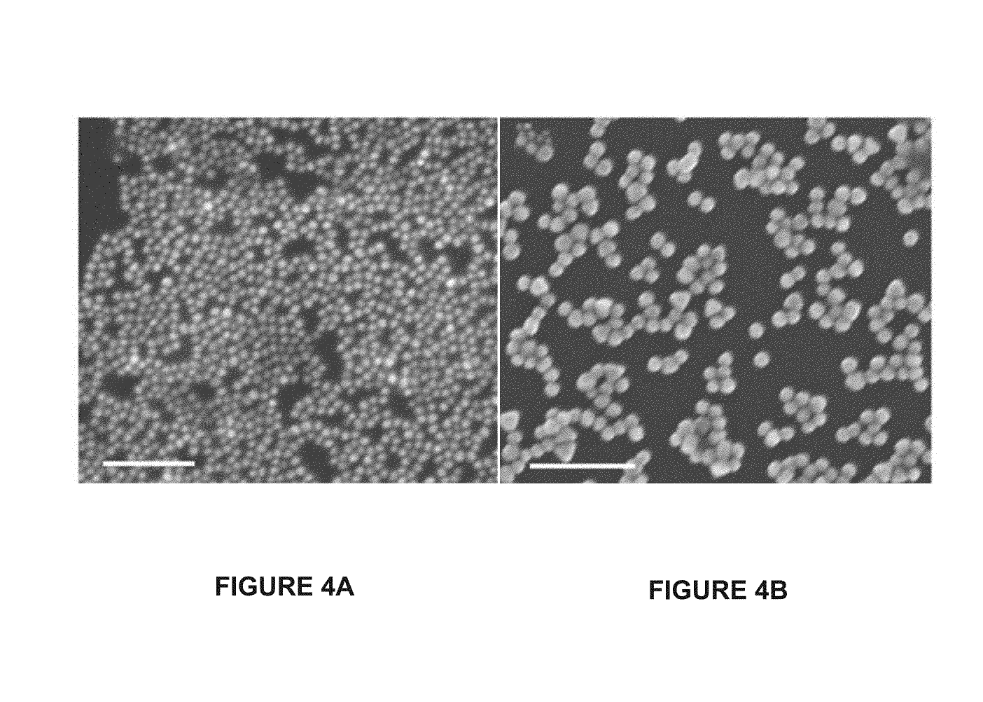

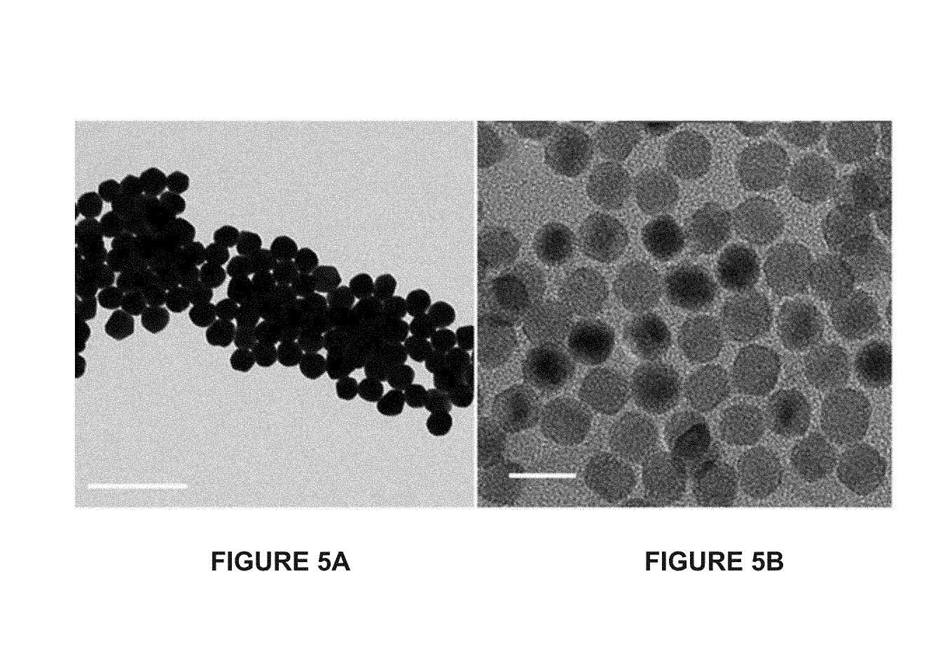

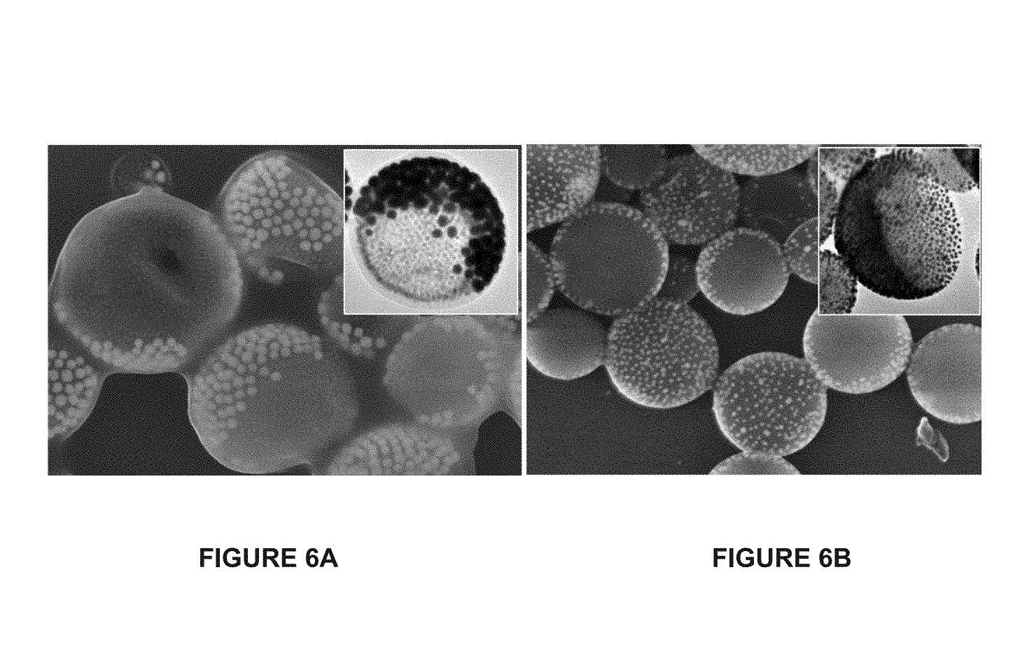

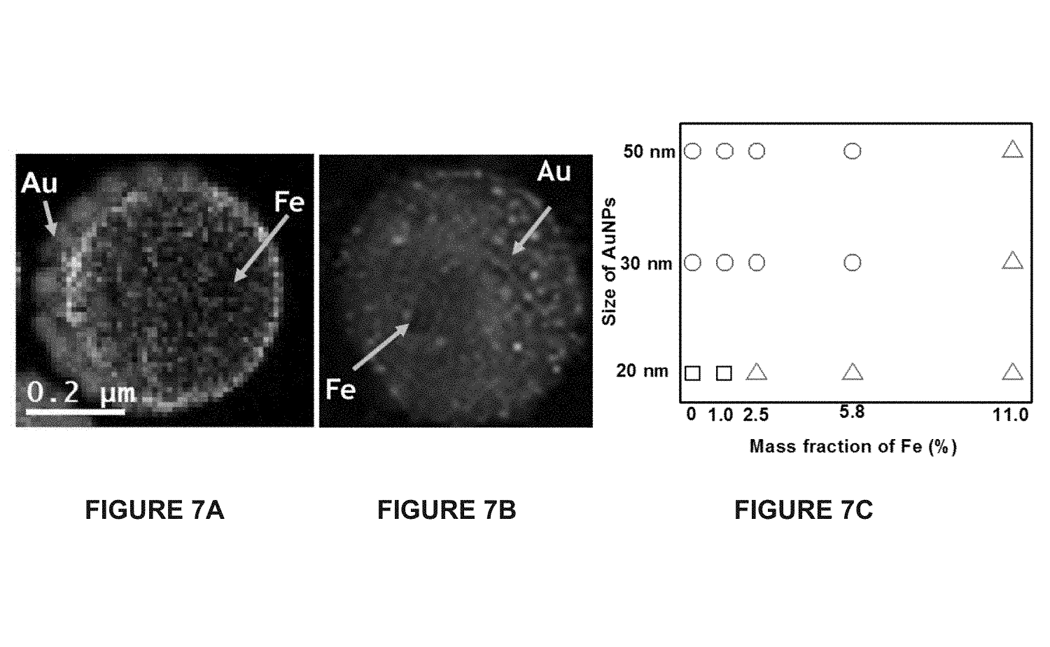

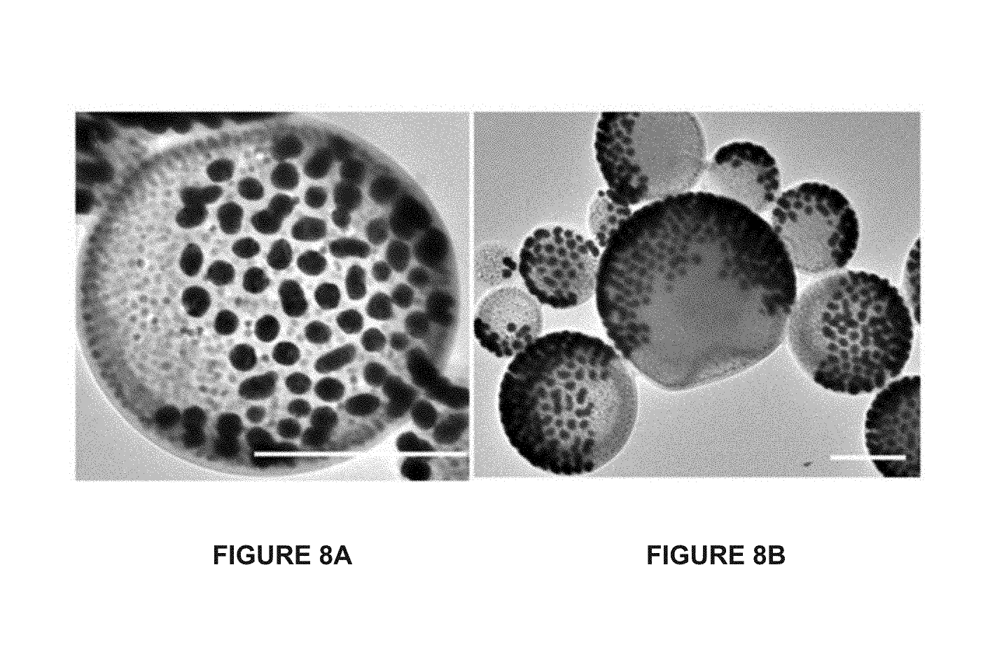

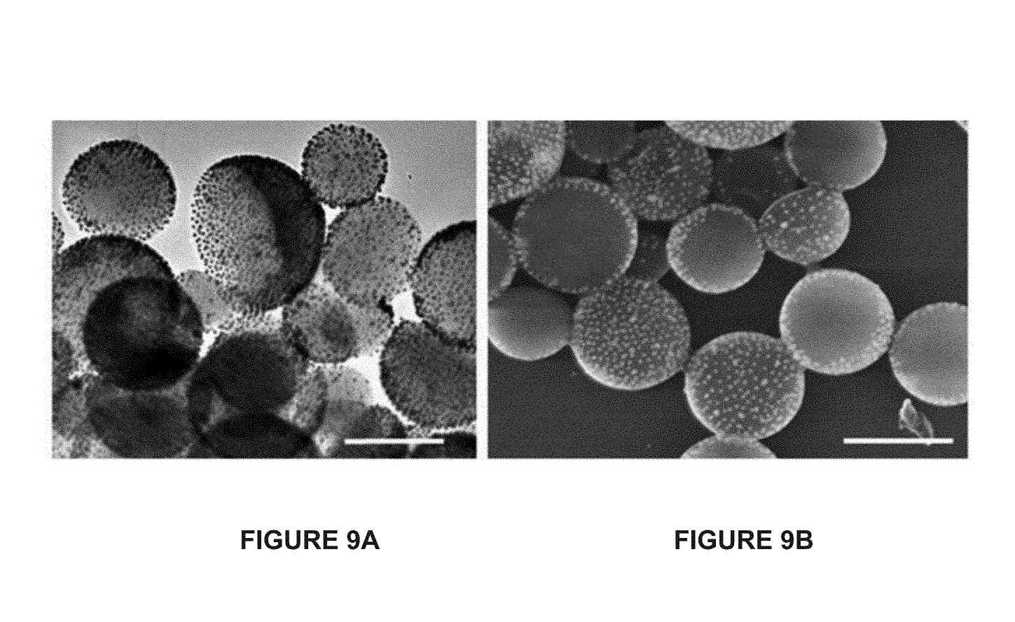

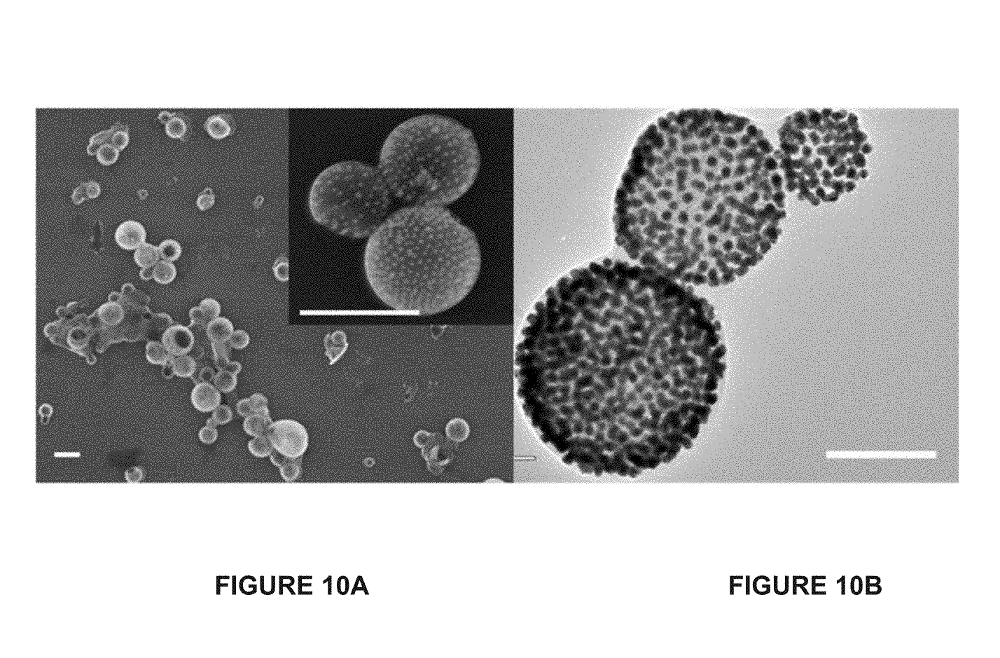

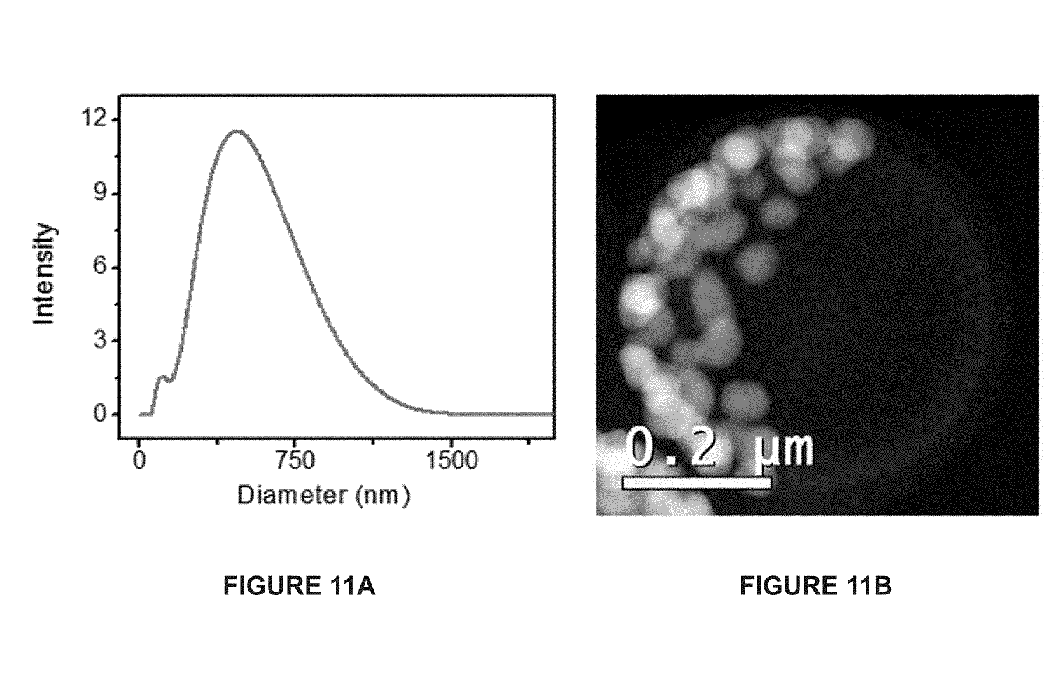

Two different sized hydrophobic Fe₃O₄ MNPs (25 nm and 15 nm), stablized by oleic acid, were used. BCP-tethered AuNPs were made by attaching thiol terminated PS₄₉₀-b-PEO₄₅ onto the surfaces of NPs with different sizes (20 nm, 30 nm, and 50 nm) (FIGS. 4A, 4B, 5A, and 5B). BCPs of PS₁₀₇-b-PAA₄ without thiol groups were used as free BCPs. The self-assembly of ternary mixture was triggered by solvent exchange method. Depending on the size and mass fraction of NP building blocks, the assembly process produced spherical and hemispherical JVs with two distinct halves and homogenous vesicles (HVs) with uniform distribution of two types of NPs (FIGS. 6A, 6B, 8A, 8B, 9A, 9B, 10A, and 10B). All JVs constitute a hollow cavity and a membrane composed of BCP-tethered AuNPs, MNPs, and free BCPs, as indicated by the higher contrast at the edges and the wrinkle surface—which are two typical characteristics of vesicles —of the assemblies in TEM images (FIGS. 6A (inset), 6B (inset), 8B, and 9B). The average diameter of the JVs was 570.8±93.2 nm, characterized by dynamic light scattering (FIG. 11A). Within the vesicular membranes of JVs, the BCP-tethered AuNPs were segregated and densely packed in one half of the vesicular membrane, while the hydrophobic MNPs could be clearly observed in the polymeric domains in another half of the vesicles. A close inspection revealed the presence of some MNPs between BCP-tethered AuNPs (FIG. 8A). High angle annular dark field (HAADF) STEM (FIG. 11B) and energy dispersive X-ray spectrometry (EDS) showed that iron was distributed on the entire vesicular membrane and Au was only observed in one half of both vesicles (FIGS. 7A and 7B). Hemispherical JVs had a bowl-like vesicular body containing a mixture of BCP-tethered AuNPs and MNPs, covered by a flat polymeric membrane containing MNPs only (FIG. 6A). In HVs, BCP-tethed AuNPs and MNPs were distributed in the entire polymeric membrane of hybrid vesicles (FIGS. 10A and 10B).

The effect of size of BCP-tethered AuNPs (or AuNP cores) and mass fraction of MNPs in the mixed building blocks on the assembly morphology was investigated. The results were summarized in a phase-like diagram (FIG. 7C). A transition from spherical to hemispherical shape of vesicles was observed when the mass fraction of MNPs in the mixed building blocks increased. It is presumed that the structural transition is a result of phase separation between BCP-tethered AuNPs and free PS-b-PAA and the increase in packing parameter of PS-b-PAA upon the addition of MNPs.

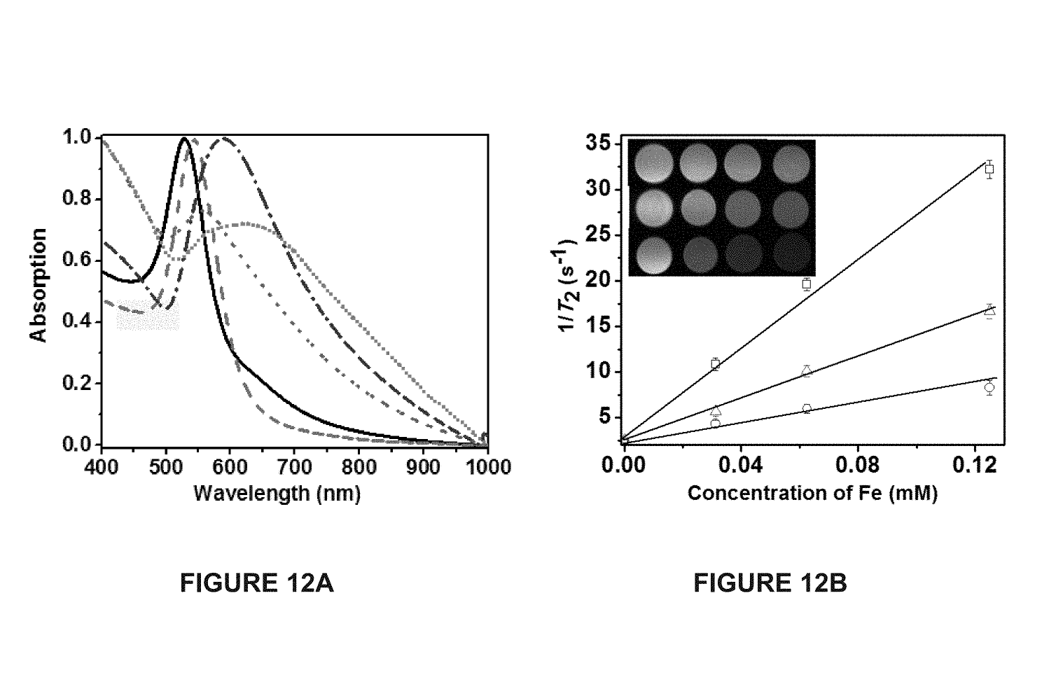

The organization of both NPs in the vesicular membranes influences the optical and magnetic properties of hybrid vesicles. For the 50 nm AuNP system, the hemispherical JVs showed a larger shift in the absorption from 543 nm of individual NPs to a broad peak in the range of 600 to 700 nm (FIG. 12A), which was slightly more than spherical JVs. The red-shift in the plasmon peak is proportional to e^{(-d/D)}, where d and D are interparticle distance and NP diameter, respectively. Thus, the larger redshift for hemispherical JVs than spherical JVs can be attributed to the more dense packing of AuNPs in the vesicular membrane.

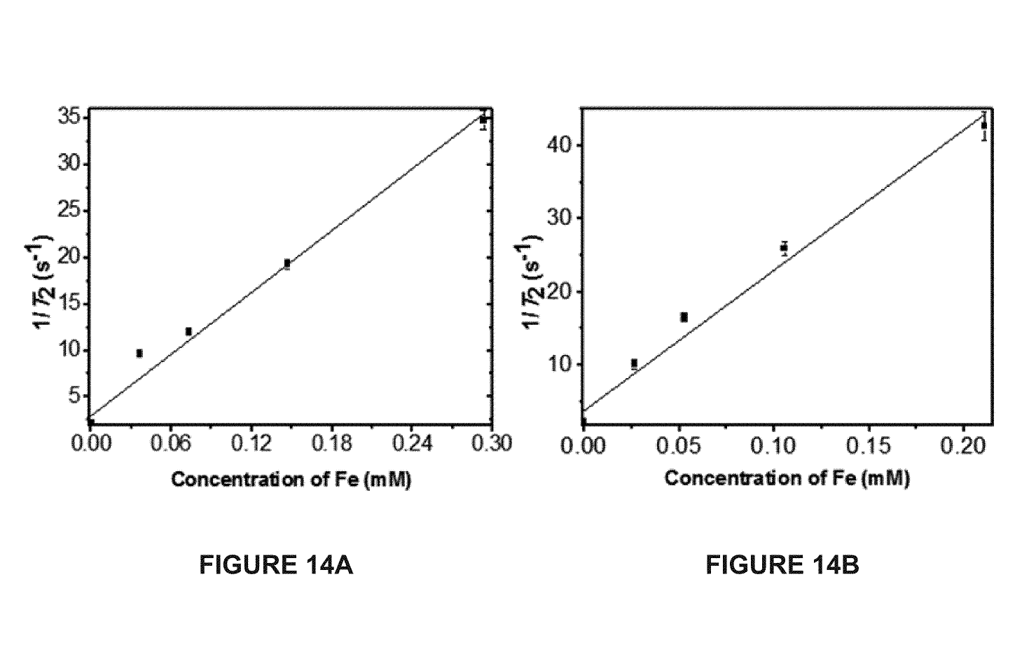

The r₂ of spherical and hemispherical JVs containing 50 nm AuNPs and 15 nm MNPs was compared with that of single MNPs. The r₂ of hemispherical JVs was measured from transverse relaxation (T₂)-weighted MR images (FIG. 12B inset) and was calculated to be 239.6 s-1 mM-1. The value was substantially higher than 114.5 s⁻¹ mM⁻¹ of spherical JVs and 47.8 s⁻¹ mM⁻¹ of single MNPs (FIG. 12B). The drastic increase in r₂ value can be attributed to the increased number of MNPs in individual hemispherical JVs compared with spherical JVs. A smilar trend of r₂ increase with increasing Fe concentration were also observed when 20 nm AuNPs were used (FIG. 14B).

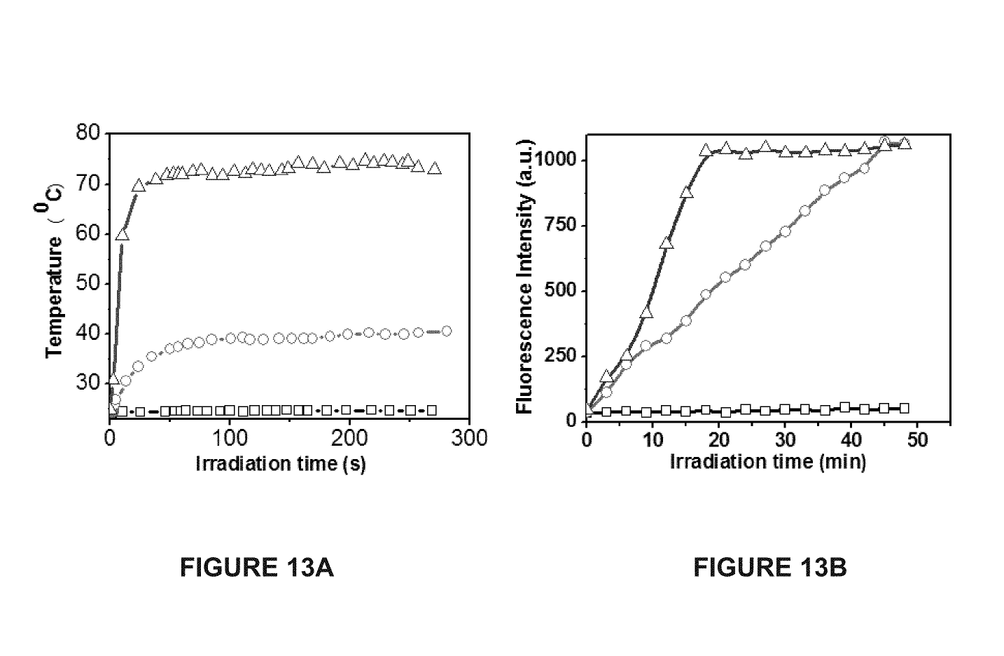

The magnetic manipulation of JVs can enrich the materials at a target location and hence drastically enhance the localized photothermal (PT) heating. The increase of localized temperature at one spot of the JV solution in a capillary tube before and after applying a magnetic field was compared, when the solution was irradiated with a laser (655 nm, 0.35 W/cm²). The localized temperature of vesicle solution increased from 24° C. to 40° C. and 70° C. in 4.5 minutes, respectivly (FIG. 13A). The faster temperature increase for the group with magnetic field was ascribed to a more rapid heating due to higher concentration of locally-enriched Au materials and relatively slow heat dissipation to surrounding water.

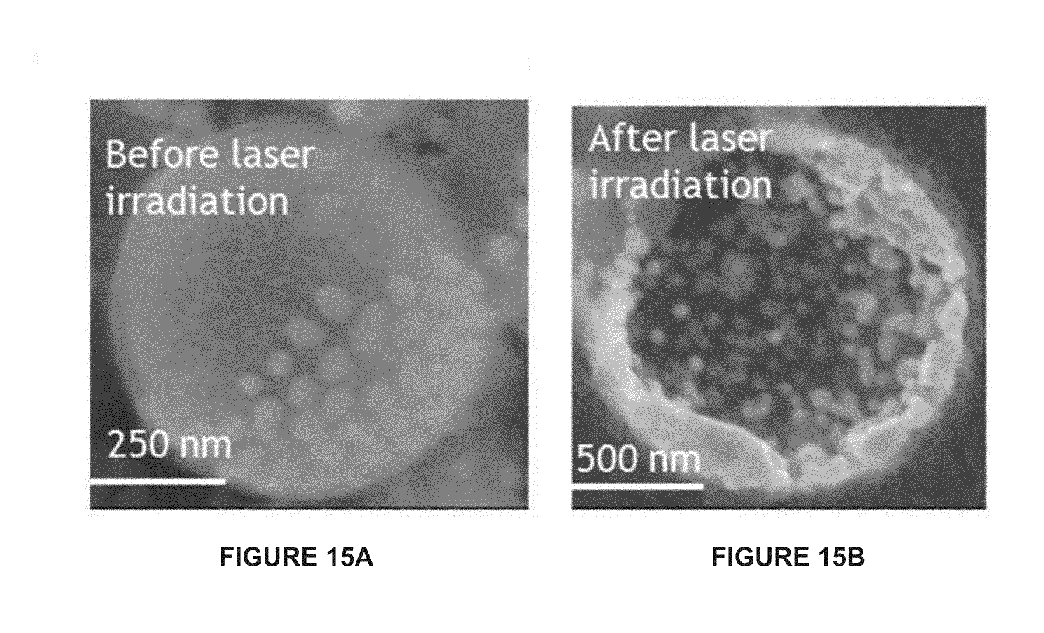

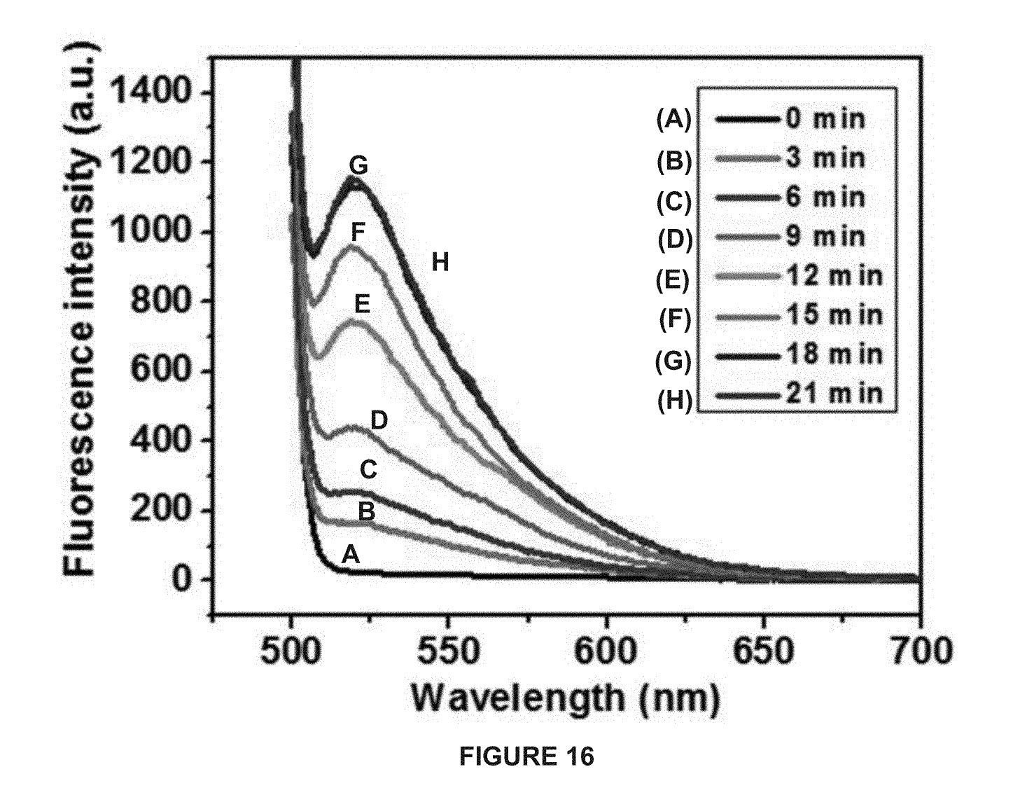

To demonstrate the potential use of JVs in remote-controlled release of payloads, a model drug, fluorescein isothiocyanate (FITC), was encapsulated in the JVs during the assembly process. The localized PT heating melted the AuNPs and broke the integrity of the vesicles, leading to the release of payload (FIGS. 15A and 15B). FIG. 13B shows that the fluorescence intensity at 520 nm increased almost linearly as a function of irradiation time and reached a plateau in 45 min (FIG. 16). When an external magnetic field was applied to concentrate the vesicles, the release rate of FITC from vesicles drastically increased under the same laser irradiation (FIG. 13B). By controlling the light source, a more sustained release over a longer period could be achieved.

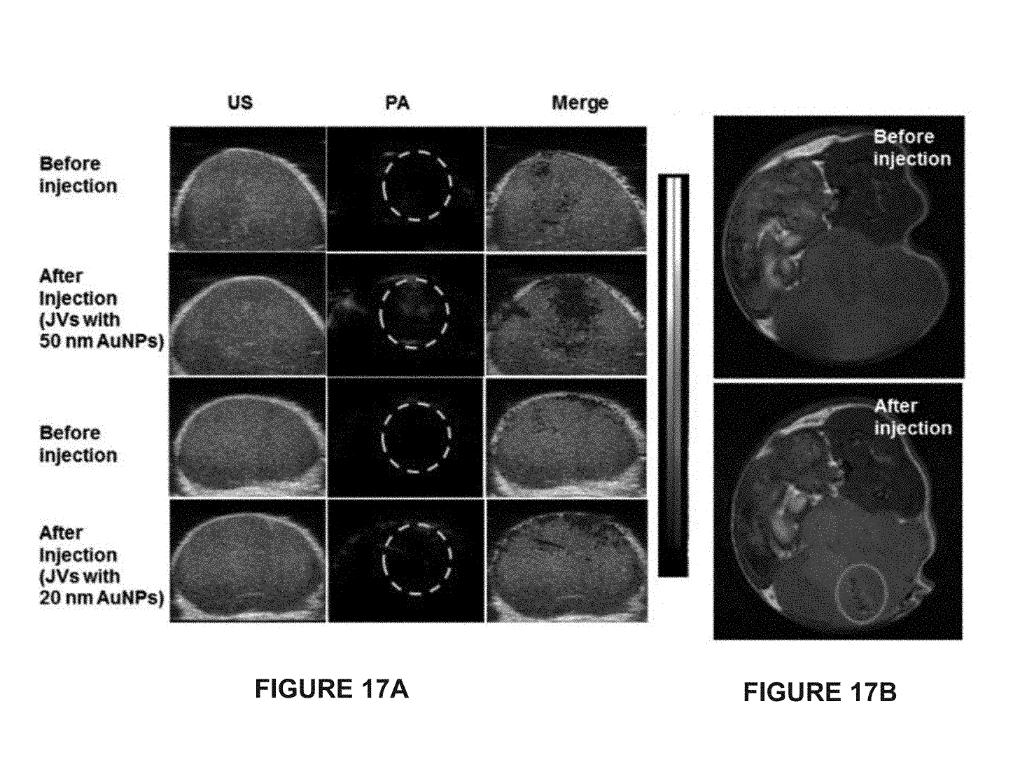

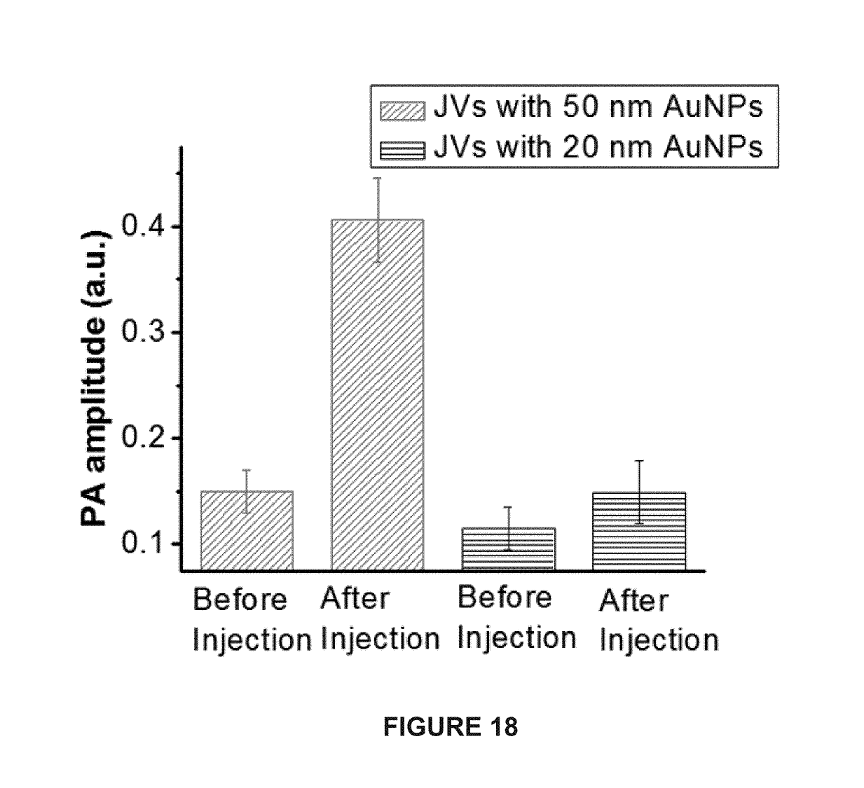

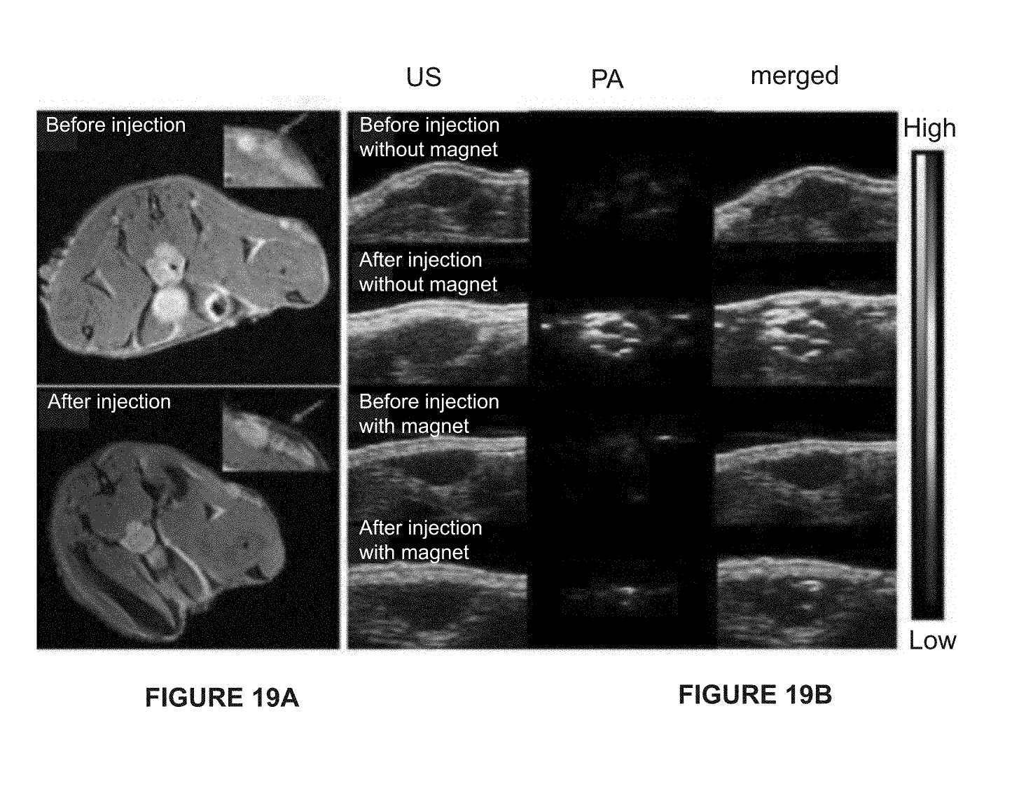

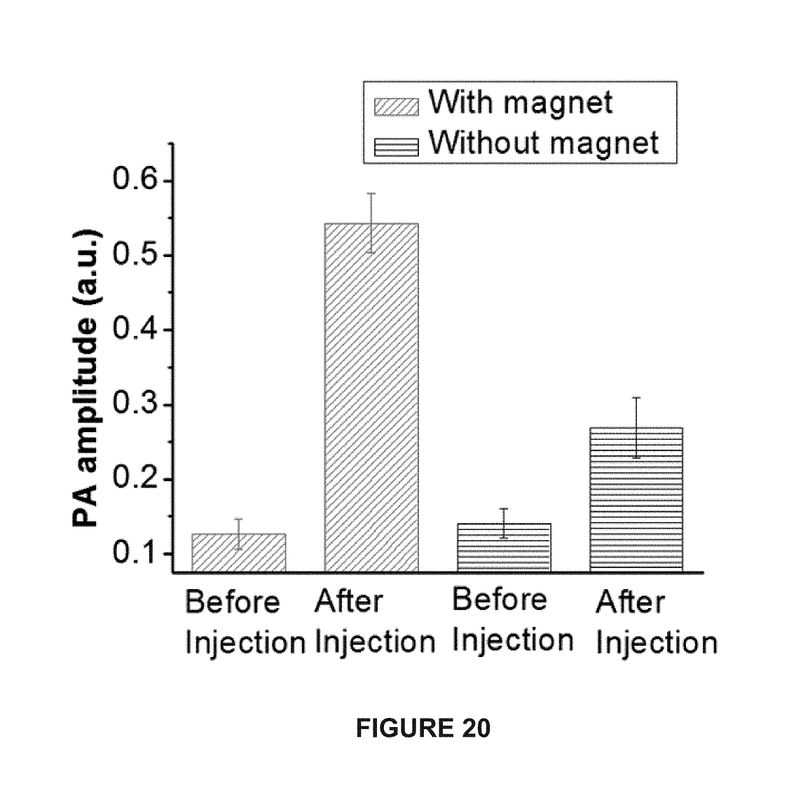

The use of magneto-plasmonic JVs as contrast agents for in vivo bimodality PA and MR imaging was investigated. Due to their stronger plasmonic coupling (FIGS. 17A, 17b, and 18), hemispherical JVs containing 50 nm AuNPs and 15 nm MNPs were chosen and administrated intravenously into athymic nude mice bearing U87MG tumors on the hind leg. In the experimental group, a magnet was attached to the hind leg of tumor-bearing mice that were intravenously injected with hemispherical JVs (the total amount of AuNPs and MNPs of injected JVs was 160.0 μg and 20.0 μg, respectively), while the control group was identical to the experimental group except no magnet was applied. The tumors were imaged by PA and MR techniques before and two hours after the injection of the JVs. In presence of magnet, a significant darkening (49.3% from the baseline) in tumor was observed from the T₂-weighted contrast images of tumors obtained before and two hours after injection of JVs (FIG. 19A). By contrast, in absence of a magnet, only 18.6% darkening from the baseline was observed (FIG. 19A). For PA imaging, the tumors were exposed to a pulsed 700 nm NIR laser, at the same value of optical density. With the assistance of external magnetic field, the PA signals in tumors were 4.3 times greater than that before injecting JVs (FIGS. 19B and 20). However, without magnets, only 1.9 times of PA signal enhancement was observed before and after injection of JVs (FIGS. 19B and 20).

In summary, Janus-like magneto-plasmonic hybrid vesicles with both spherical and hemispherical shapes through co-assembly of multiple types of building blocks were successfully prepared. The hemispherical JVs have a strong NIR absorption and a higher r₂ than their spherical counterparts. The JVs can encapsulate therapeutic compounds and the release rate of the payload can be remotely controlled by NIR light and external magnetic field. The effective enrichment of intravenously-injected JVs with external magnetic field could drastically enhance the PA and MR imaging signals in tumors.

### Example 2

### Materials for Examples 3-11

Tetrahydrofuran (THF), N,N-dimethylformamide, (DMF) gold(III) chloride trihydrate (HAuCl₄, ≥99.9% trace metals basis), sodium citrate tribasic dihydrate (≥99%), sodium oleate (NaOL), silver nitrate (AgNO₃), iron(III) chloride hexahydrate (FeCl₃.6H₂O), 3,4-dihydroxyhydrocinnamic acid, hexane, sodium hydroxide (NaOH), oleic acid, dioxane, and octadecene were purchased from Sigma-Aldrich. Free block copolymers (BCPs) of PS₁₀₇-b-PAA₄ were purchased from Polymer Source. The polymer ligands of PS₄₉₀-b-PEO₄₅ were prepared by the reversible addition—fragmentation chain transfer polymerization.

### Example 3

### Synthesis of Inorganic Nanoparticles

Magnetic nanoparticles (MNPs) were prepared using the method described in Park, J., et al., Nat. Mater. 3:891-895 (2004). Iron oleate, the precursor for the MNP synthesis, was synthesized as follows. FeCl₃.6H₂O (5.4 g) and NaOL (18.25 g) were dissolved in a solvent mixture containing 40 mL ethanol, 70 mL hexane, and 30 mL distilled water. The solution was stirred at 70° C. for 4 hours before the organic layer was extracted. The organic solution was washed with water 3 times. Then organic solvents were removed through rotary evaporator and vacuum oven. For the synthesis of MNPs, iron-oleate (9 g), oleic acid (1.4 g), and 40 mL of octadecene were mixed in a 3-neck flask followed by pumping with Argon for 30 minutes. The reaction temperature was elevated to 310° C. and was maintained for 30 minutes. After purification, MNPs were dissolved in THF for self-assembly.

Gold NPs (AuNPs) were synthesized using the sodium citrate reduction method described in He, J., et al., J. Am. Chem. Soc. 134:11342-11345 (2012). AuNP seeds were first prepared by injecting 1 mL of 10 mg/mL HAuCl₄ aqueous solution and 3 mL of 10 mg/mL sodium citrate into 500 mL of boiling water under stirring. After refluxing for 30 minutes, the solution temperature was decreased to 85° C. Another 3 mL of sodium citrate solution and 1 mL of HAuCl₄ solution were injected. This procedure was repeated until the AuNPs reached the desired size.

### Example 4

### Preparation of Block Copolymer-Tethered AuNPs

Block copolymer-tethered AuNPs were prepared using the ligand exchange method described in Liu, Y., et al., J. Am. Chem. Soc. 136:2602-2610 (2014). Thiol-terminated PS₄₉₀-b-PEO₄₅ (3 mg) was dissolved in 10 mL of DMF. A concentrated aqueous solution of AuNPs (4 mg of AuNPs) was dropwise added into the above polymer solution under sonication. The block copolymer-tethered AuNPs were centrifuged and washed 6 times with THF to remove unbounded block copolymer. The functionalized AuNPs were dispersed in THF and the final concentration of AuNPs in THF was adjusted to 3 mg/mL.

### Example 5

### Self-Assembly of Magneto-Plasmonic Janus Vesicles

Both MNPs and free block copolymers were dispersed/dissolved in THF. The concentration of MNPs and free block copolymers were 10 mg/mL and 0.8 mg/mL, respectively. For the self-assembly of hemispherical Janus vesicles (JVs) with 50 nm AuNPs, a 250 μL THF solution of block copolymer-tethered AuNPs (3 mg/mL), a 75 μL THF solution of free block copolymers (0.8 mg/mL), and a 10 μL THF solution of MNPs (10 mg/mL) were mixed together. The volume of the solution was adjusted to 400 μL by adding THF. A THF/water solvent mixture (3/2 by volume) was injected into the above solution by a syringe pump at rate of 2 mL/hour until the final water content reached 25%. The solution was held for 3 hours before being dialyzed against pure water to remove the organic solvent.

### Example 6

### Self-Assembly of MNP Micelles

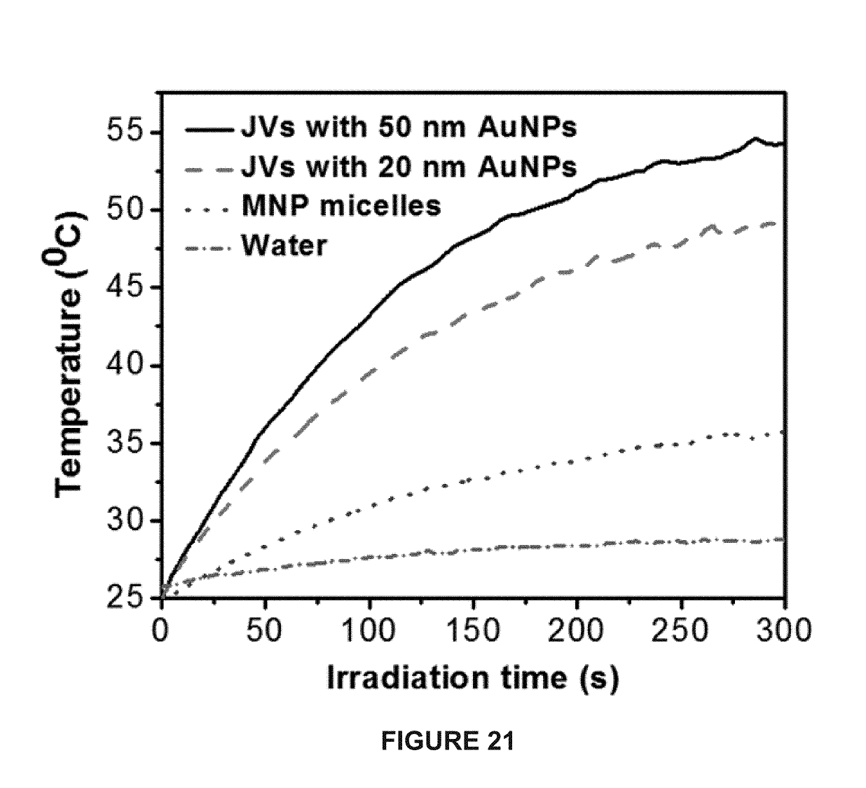

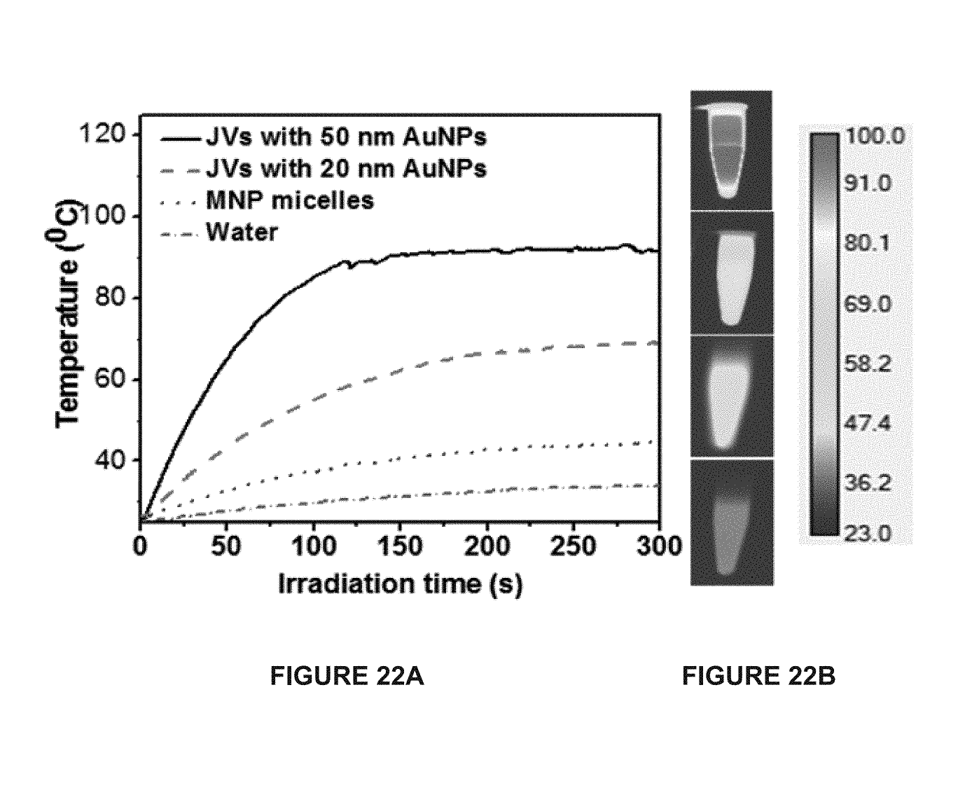

To prepare the MNP micelles as control in photothermal characterizations, a 75 μL THF solution of block copolymers and a 10 μL of NNPs solution were mixed together and the final volume was adjusted to 400 μL by adding THF. A THF/water solvent mixture (3/2 by volume) were injected into the above solution by a syringe pump at rate of 2 mL/hours until the final water content reached 25%. The solution was held for 3 hours before being dialyzed against pure water to remove the organic solvent. The magnetic micelles were used as control in the photo thermal experiment described below (FIGS. 21, 22A, and 22B).

### Example 7

### Encapsulation and Release of a Model Drug

Fluorescein isothiocyanate (FITC) was used as a model drug and encapsulated in the hybrid JVs during the assembly process. A THF solution of FITC (0.1 mg/mL) was added to a mixture containing BCP-tethered AuNPs, MNPs, and free BCPs to make a final volume of 400 μL and self-assembly followed the procedure in Example 5. The vesicle solution was dialyzed against pure water for 2 days to remove the un-encapsulated FITC. The resulting FITC loaded JVs were put into a dialysis tube and exposed to a laser (655 nm 0.35 W/cm²) at a time interval of 3 minutes. The dialysis tube was placed in a 3 mL water reservoir and 1 mL of water from reservoir was taken and measured of its fluorescence intensity by fluorescence spectrometer every three minutes. After each measurement, the 1 mL water was put back into the water reservoir.

### Example 8

### Transferring of Individual Hydrophobic MNPs Into Water

Water soluble individual MNPs were used as control compared with hybrid vesicles in the measurement of transverse relaxivity. As-synthesized individual hydrophobic MNPs were transferred into water using the method described in Liu, Y., et al., J. Am. Chem. Soc. 136:12552-12555 (2014). The ligand of 3,4-dihydroxyhydrocinnamic acid and MNPs were mixed in THF and the solution was sonicated for 3 hours. Then, the NPs were precipitated by adding NaOH (aqueous) and the supernatant was removed through centrifugation. The final product was dispersed in water for future use.

### Example 9

### Characterization of NPs and Assemblies

The assembled structures were imaged using a Hitachi SU-70 Schottky field-emission gun (FEG) Scanning Electron Microscope (SEM) and a JEOL FEG Transmission Electron Microscope (TEM). SEM samples were prepared by casting a 5-10 μL of sample solution on silicon wafers and were dried at room temperature. TEM samples were prepared by casting on 300 mesh copper grids covered with carbon film and were dried at room temperature. The absorption spectra of GNPs were measured by a PERKIN LAMBDA 35 UV-Vis spectrometer. Photothermal induced solution temperature increase was measured by a SC300 infrared camera. The concentrations of Fe and Au for each sample were measured by Agilent 700 series ICP Optical Emission Spectrometers. 50 μL of sample solution was added to a 20 mL vial, followed by digestion of sample by 50 μL of aqua regia while heating. Before the solution was completely dry, 3% nitric acid was used to dilute the sample to target volume.

### Example 10

### In Vivo Photoacoustic (PA) and Magnetic Resonance (MR) Imaging Through Intratumoral Injection

All animal experiments were performed under a National Institutes of Health Animal Care and Use Committee (NIHACUC) approved protocol. A total of 2×10⁶ U87MG cells were subcutaneously injected into the right hind leg of athymic nude mice to grow into subcutaneous tumor. MR imaging was recorded on a high magnetic field micro-MR scanner (7.0 T, Bruker, Pharmascan) with small animal-specific body coil. PA imaging was performed by a Vevo 2100 LAER system (VisualSonics Inc., New York, N..Y) equipped with a 40 MHz, 256-element linear array transducer. Before the injection of materials, background from tumor tissues of both MR and PA imaging were measured. Then, 50 μL JVs containing 16 μg of AuNPs and 2 μg of MNPs were intratumorally injected and corresponding MR and PA images were obtained.

### Example 11

### Magnetic-Field-Enhanced In Vivo PA and MR Imaging of Tumor Through Intravenous Injection

A total of 2×10⁶ U87MG cells were subcutaneously injected into the right hind leg of athymic nude mice to grow subcutaneous tumor. MR and PA images were obtained through the same procedure described in Example 10. Before the injection of materials, background of both MR and PA imaging from tumor tissues were measured for both experimental and control groups. In the experimental group, 100 hemispherical JVs (composed of 50 nm AuNPs and 15 nm MNPs) containing 160 μg of AuNPs and 20 μg of MNPs were intratumorally injected and a magnet was attached to the tumor. The corresponding MR and PA images were obtained two hours after the injection. In the control groups, the same amount of samples were injected in the absence of a magnet, and the MR and PA images were taken two hours after the injection.

### Example 12

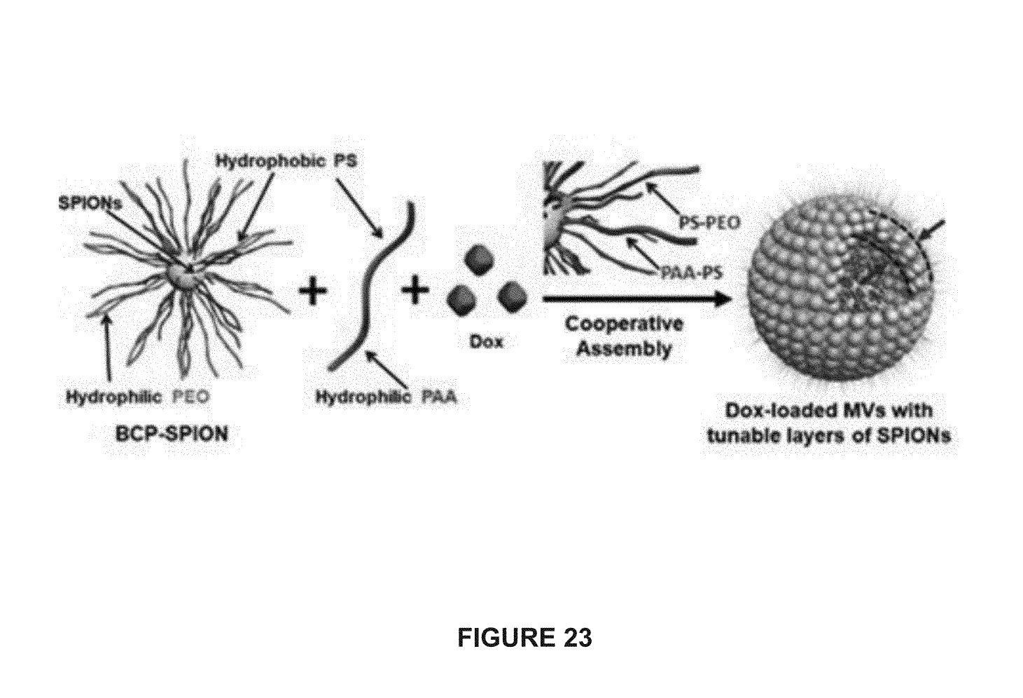

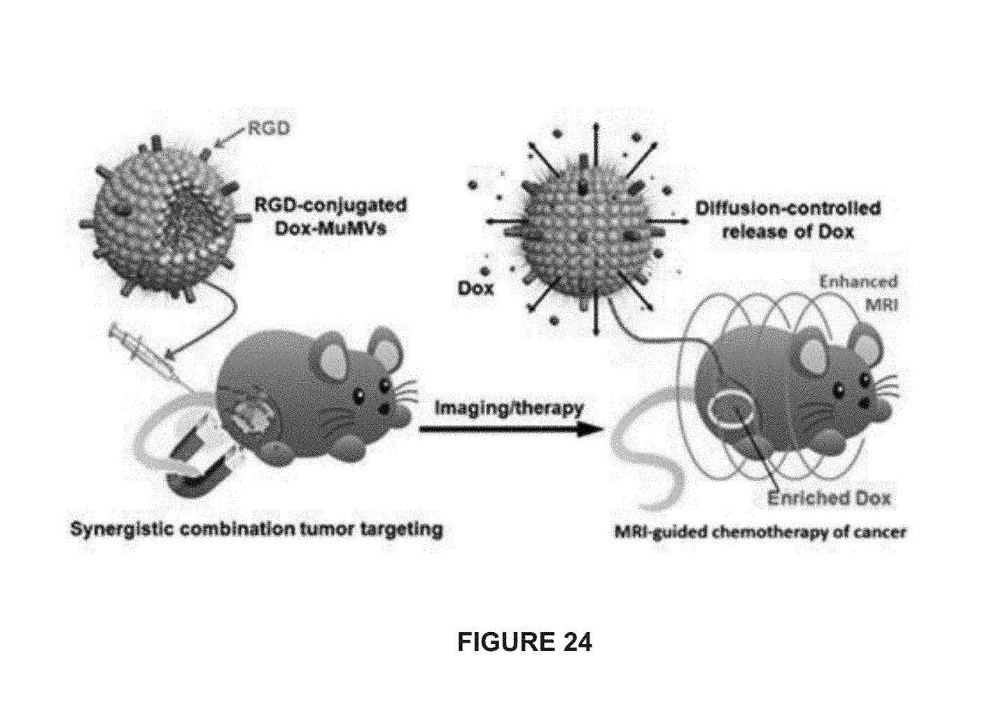

Magneto-vesicles (MVs) composed of tunable layers of densely-packed superparamagnetic iron oxide nanoparticles (SPIONs) via cooperative assembly of polystyrene-b-poly(ethylene oxide) (PS-b-PEO)-tethered SPIONs and free polystyrene-b-poly(acrylic acid) (PS-b-PAA) were designed as shown in FIG. 23. The membrane thickness of MVs can be controlled from 9.8 nm to 93.2 nm by varying the weight ratio of PS-b-PAA to SPIONs, which is accompanied with the transition from monolayer MVs (MoMVs), to double-layered MVs (DoMVs) and to multilayered MVs (MuMVs). The formation of MVs with controlled layers of SPIONs is attributed to the modulation of the surface property of SPION building blocks through the binding interaction between carboxyl groups of PS-b-PAA and SPIONs. Compared with individual SPIONs, MVs with a thicker membrane exhibit a much higher magnetization for magnetic manipulation as a result of larger amounts of SPIONs in each vesicle. As the membrane thickness of MVs increases, a higher magnetization leads to a drastically enhanced transverse relaxivity rate (r₂) value in magnetic resonance (MR) imaging due to the higher density of SPIONs. Therapeutic agents such as doxorubicin (Dox) can be efficiently encapsulated in the hollow cavity of MVs during the assembly process and the release of payload can be tuned by varying the membrane thickness of the MVs. Upon intravenous injection into athymic nude mice implanted with U87MG human malignant glioblastoma cells, the RGD-conjugated Dox-loaded MuMVs (RGD-Dox-MuMVs) exhibited significantly enhanced tumor accumulation via synergistic magnetic field-enhanced targeting and RGD-mediated active targeting of tumors (FIG. 24). As a result, RGD-Dox-MuMVs with a magnetic field showed an over ten-fold increase in the delivery of Dox in tumors and drastically enhanced tumor inhibition, compared with control groups without RGD and magnetic field.

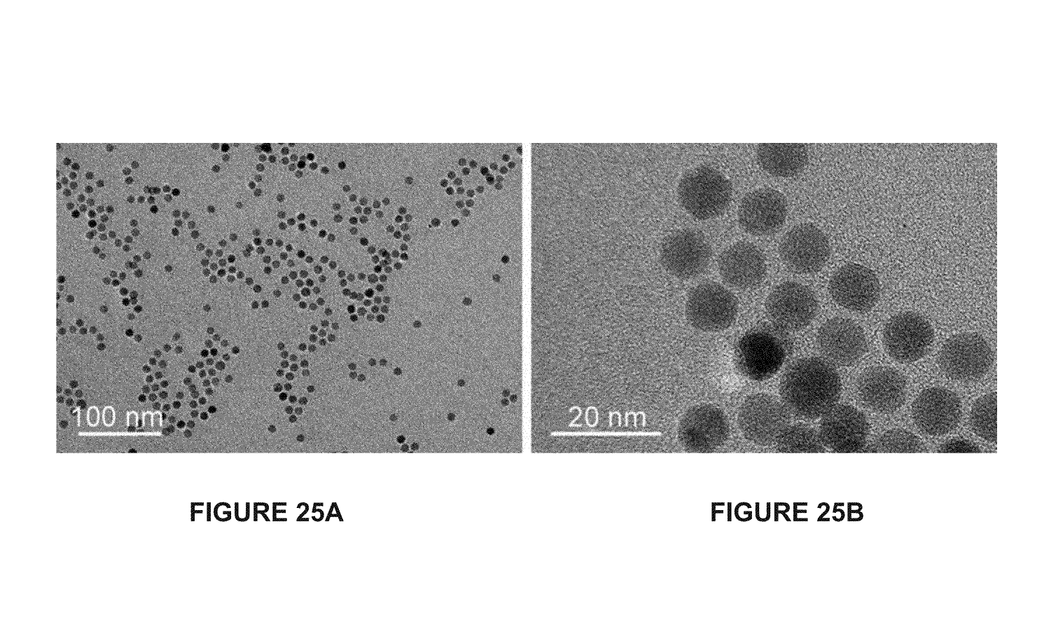

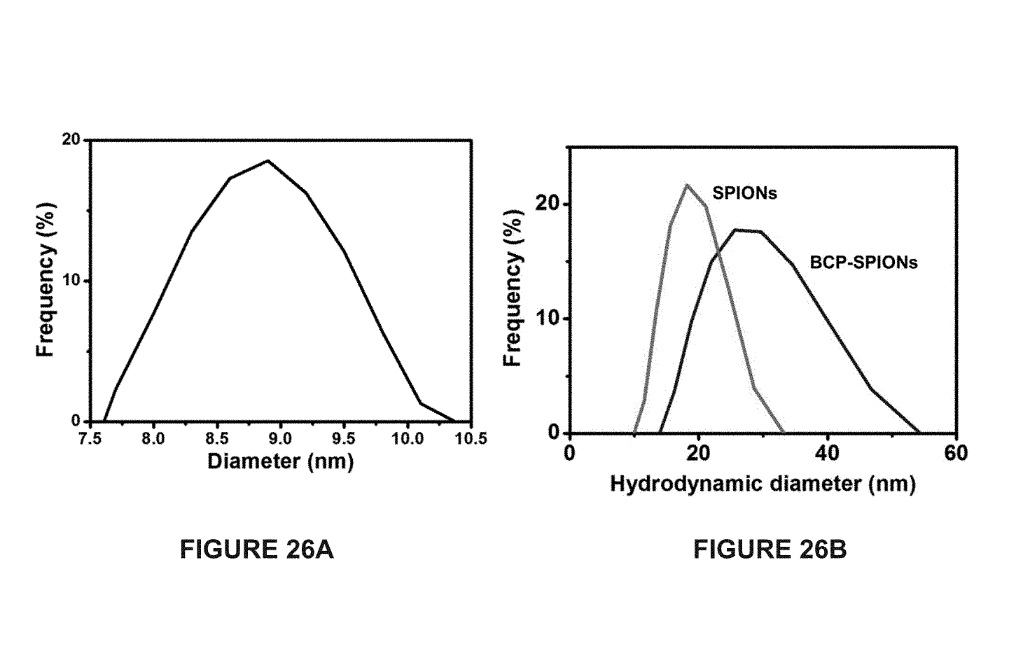

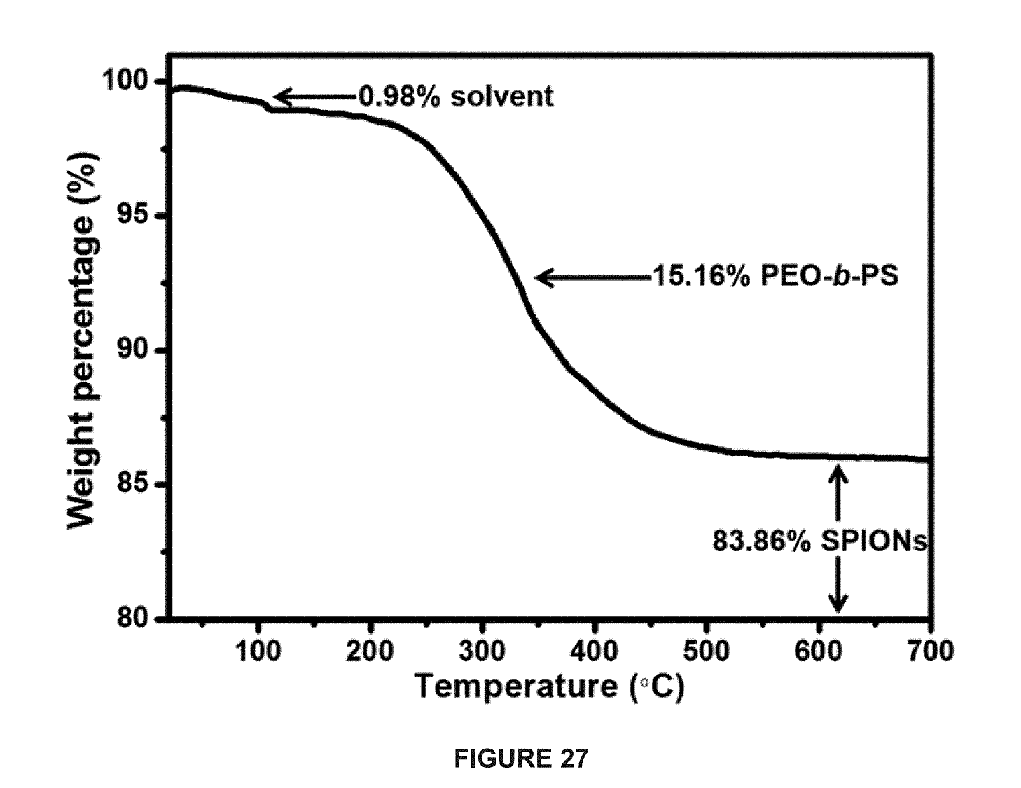

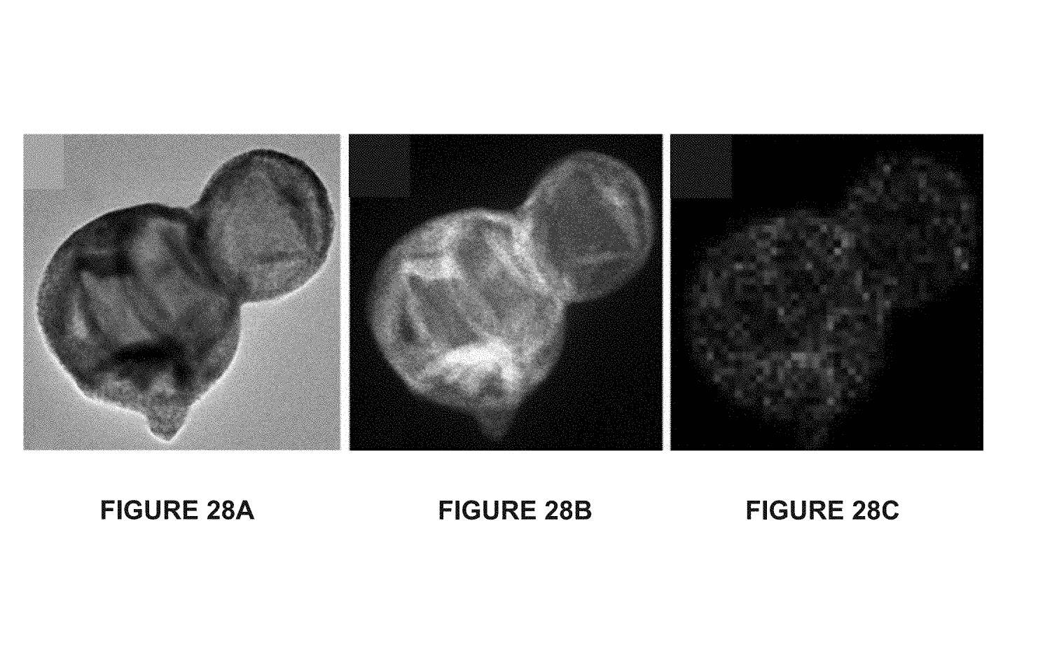

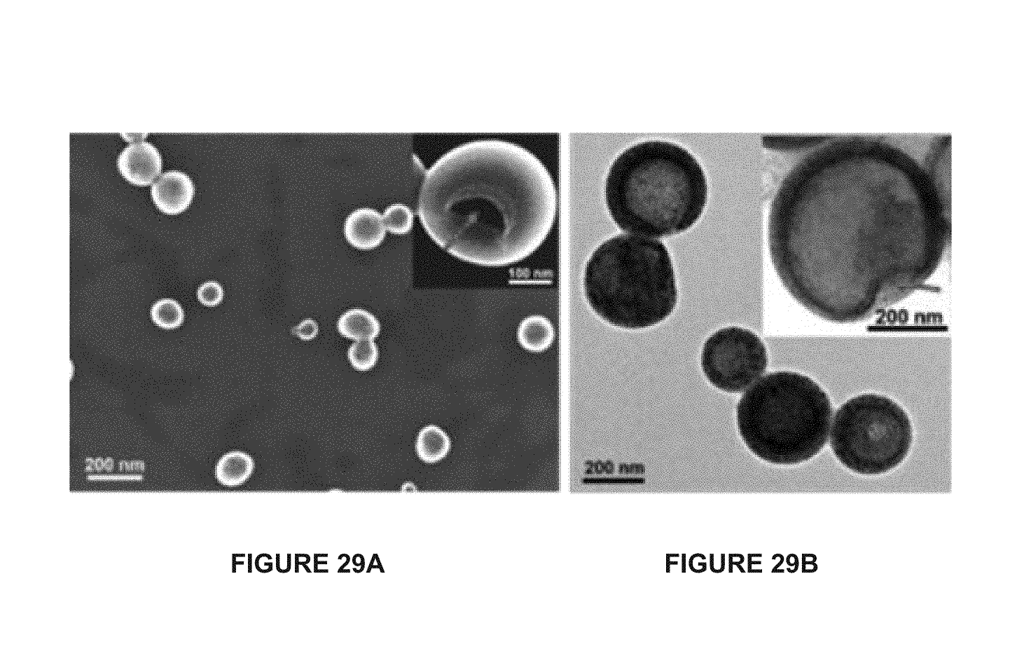

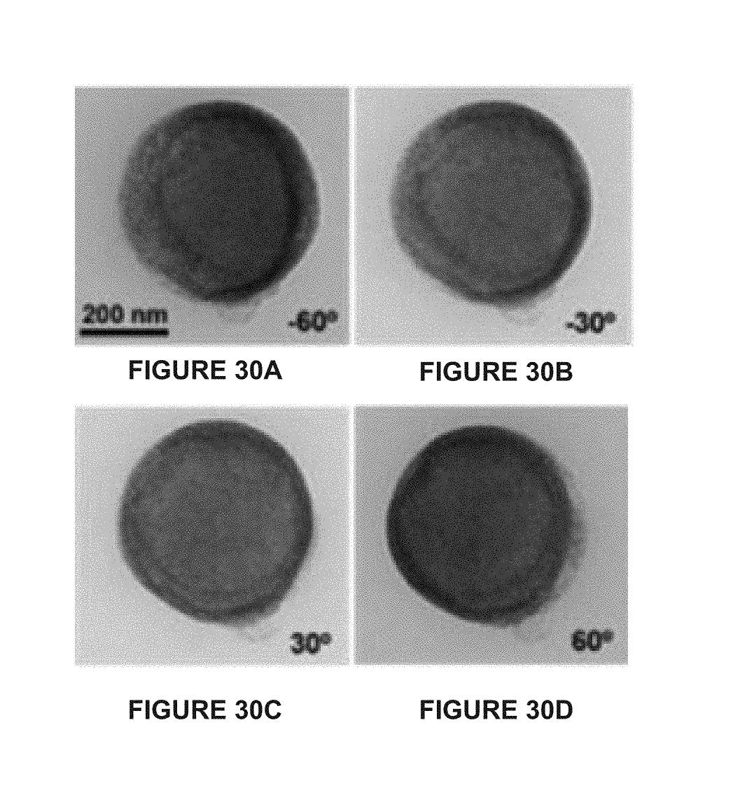

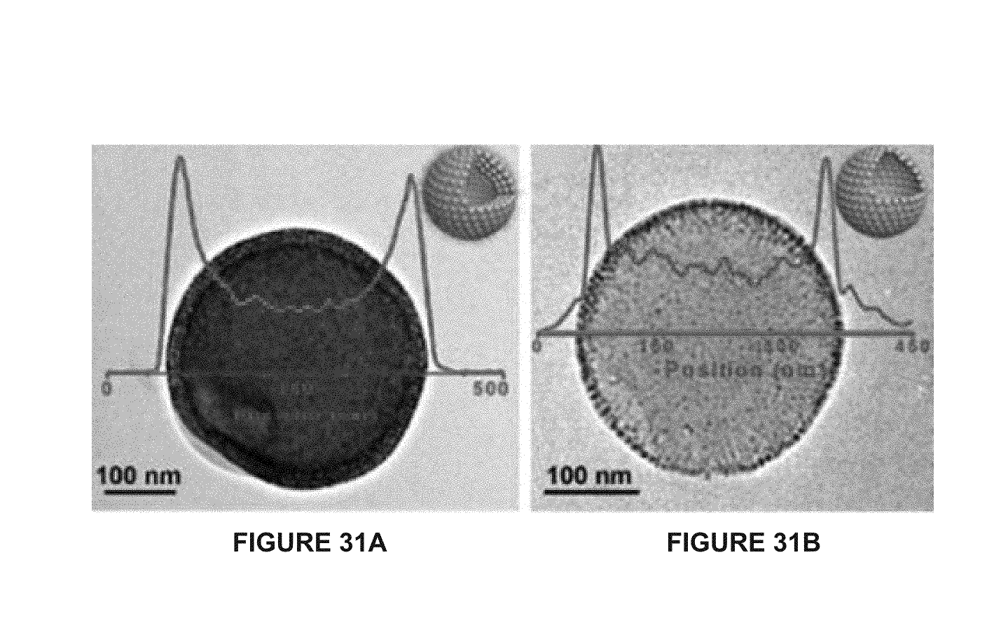

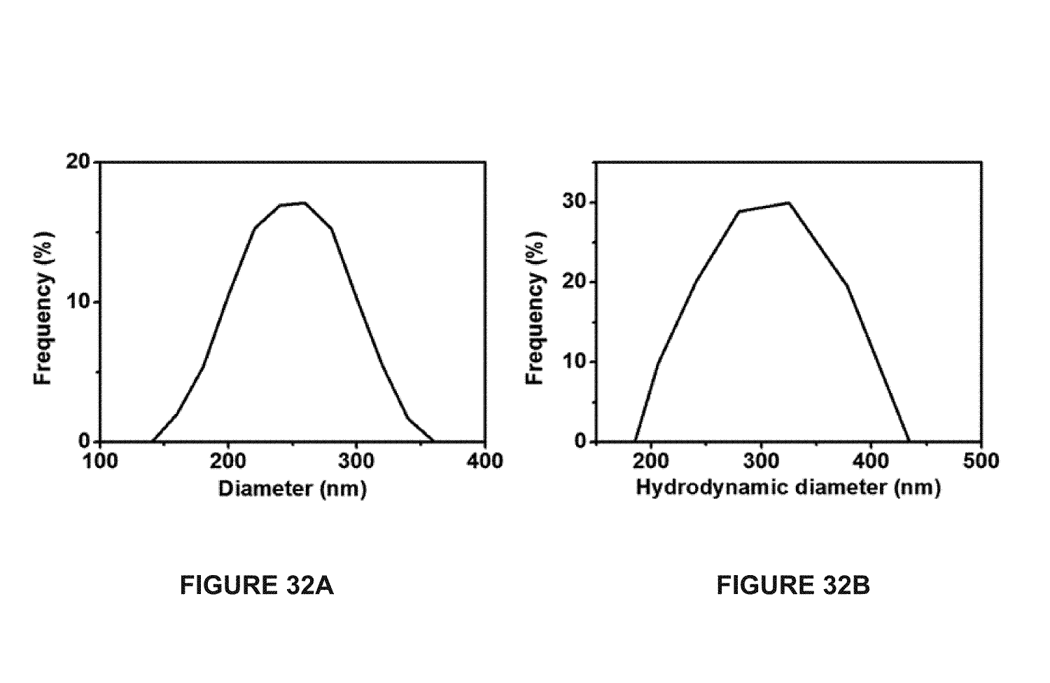

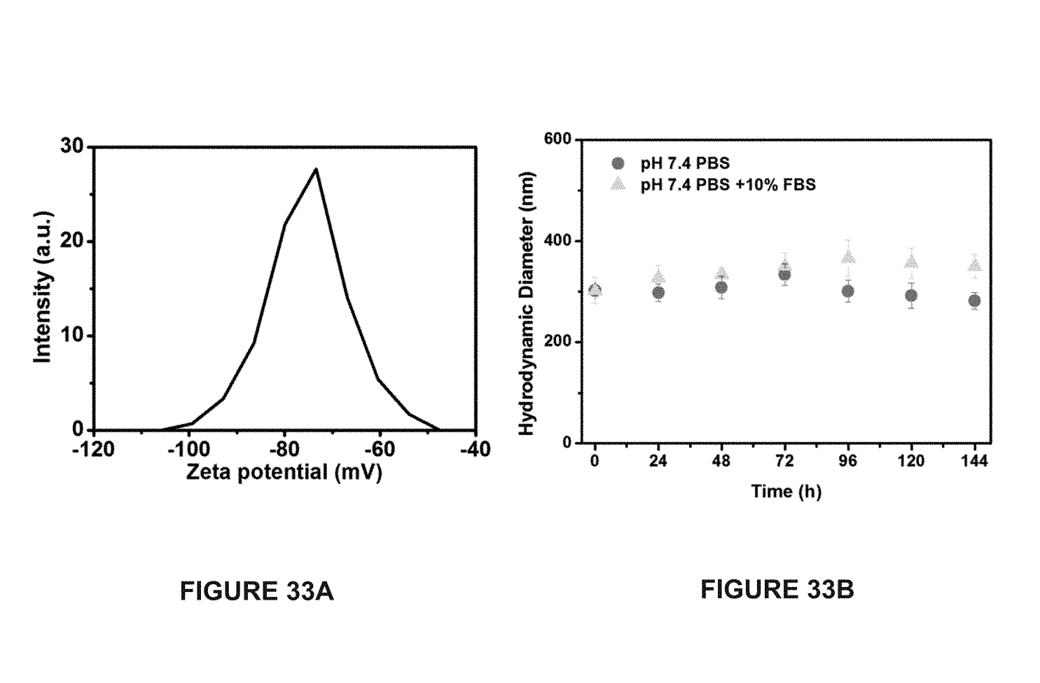

Hydrophobic SPIONs with a diameter of 9.2±0.6 nm were synthesized by a thermal decomposition method described by Park, J., Nat. Mater. 3:891-895 (2004) (FIGS. 25A, 25b, and 26A). Dopamine terminated PS₂₆₀-b-PEO₄₅ (29.0 kg/mol) was synthesized and grafted onto the surface of SPIONs to obtain amphiphilic building blocks (FIG. 26B). The average grafting density (a) of BCPs is estimated to be 0.07 chains/nm² based on thermogravimetric analysis (FIG. 27). The MVs were fabricated by rehydrating a film containing both BCP-tethered SPIONs and varying amounts of PS₁₀₆-b-PAA₄ in ultrapure water. The formation of vesicular structures can be attributed to the conformation change of BCP tethers on the NP surface. Scanning and transmission electron microscope (SEM/TEM) images in FIGS. 29A and 29B show that the resulting MuMVs were composed of multilayers of highly densely packed SPIONs in the vesicular membranes (also see FIGS. 26A-26C). The hollow interior and multilayers of SPIONs in the membrane can be clearly seen from vesicles with the occasionally broken membrane (inset in FIG. 29A). These were confirmed by TEM observations of MuMVs at different tilt angles (FIGS. 30A-30D) and three-dimensional construction of one MuMV. Moreover, the two peaks of Fe intensity corresponding to the edge of MVs were observed in the energy dispersive X-Ray spectroscopy (EDS) line scan of MVs, which further supports the formation of vesicles (FIGS. 31A and 31B). The different width of peaks in the two systems also indicates the significant difference in the wall thickness of vesicular membranes. The average diameter of the MuMVs was estimated to be 263.3±36.9 nm by TEM analysis (FIGS. 32A and 32B). The surface of MuMVs is highly negatively charged (with a zeta potential of −75.2 mV), indicating the successful integration of PS-b-PAA chains in the vesicular membranes (FIG. 33A). The MuMVs were stable for days under the physiological environment, such as in phosphate-buffered saline (PBS) and PBS supplemented with 10% fetal bovine serum (FBS) (FIG. 33B).

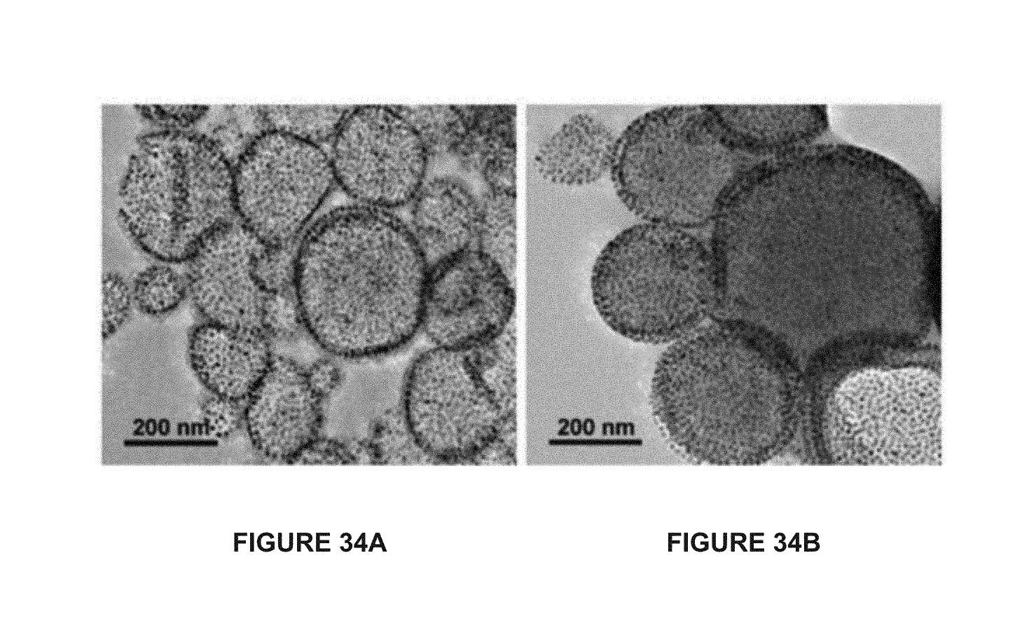

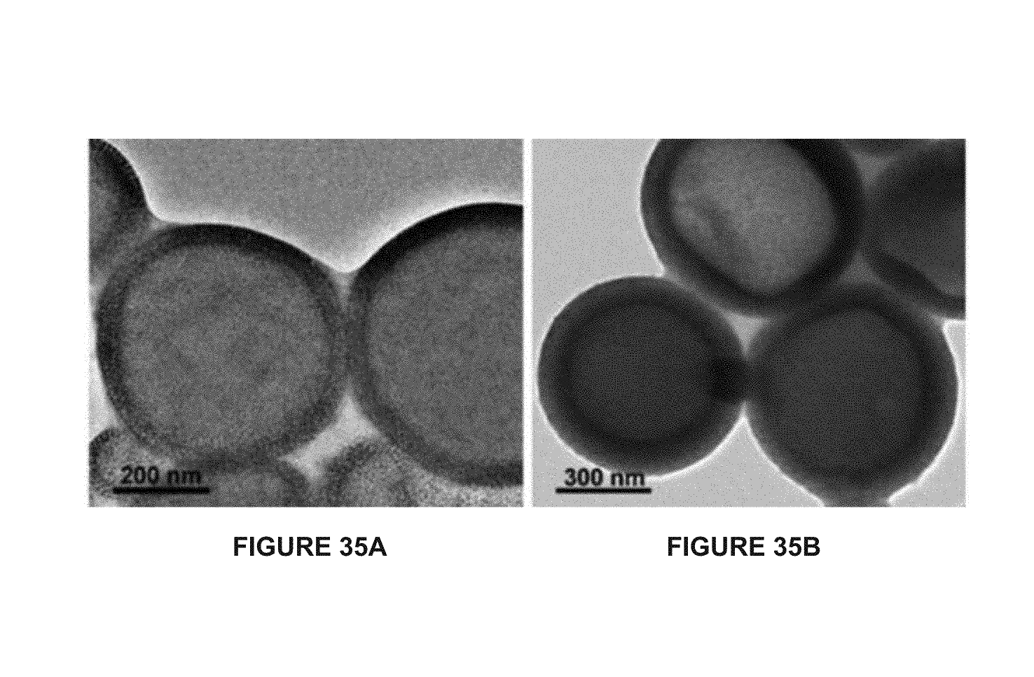

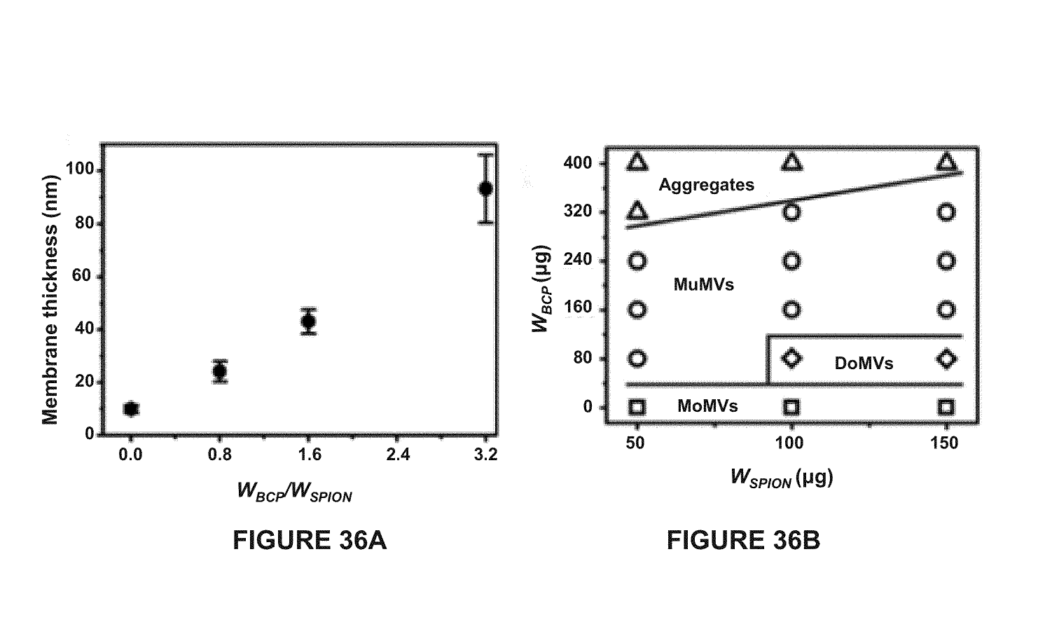

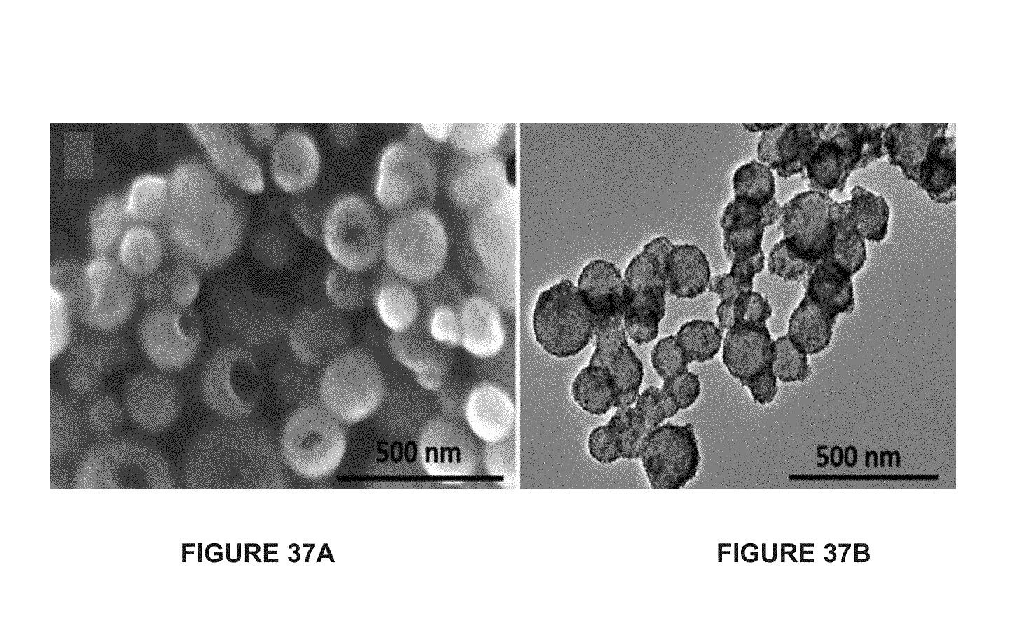

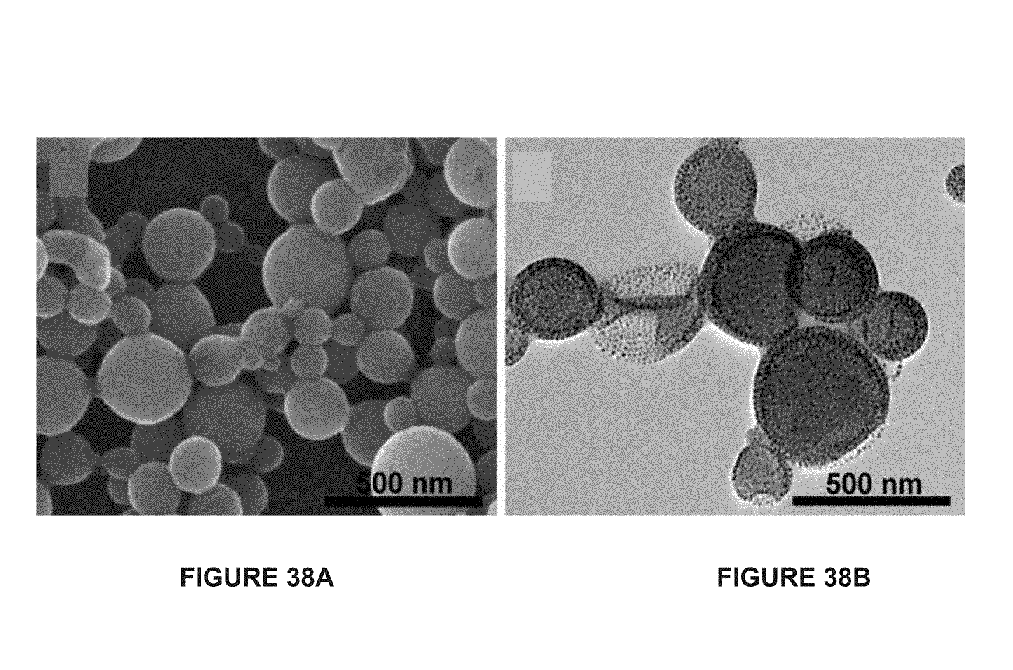

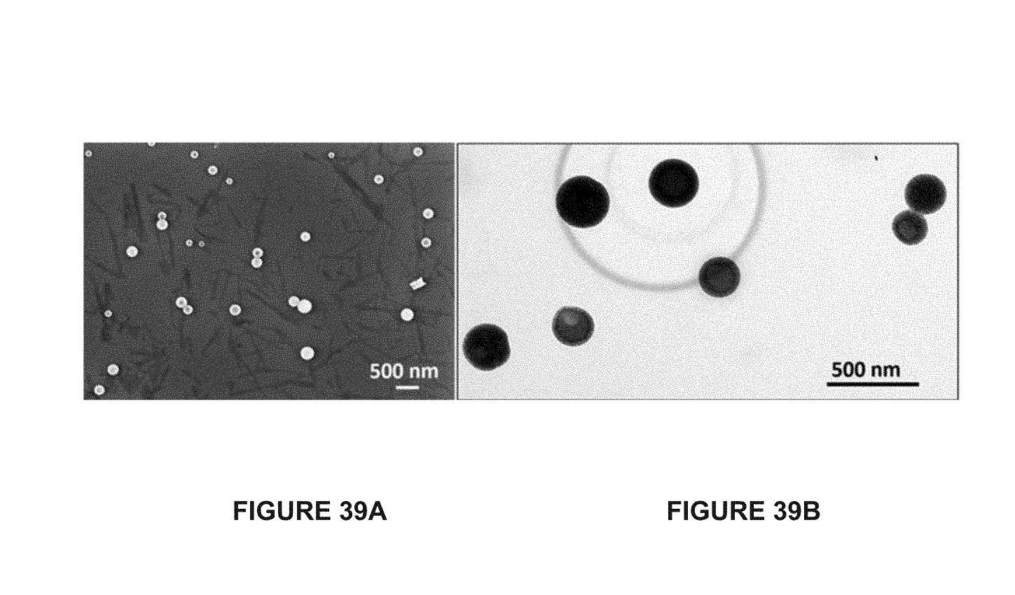

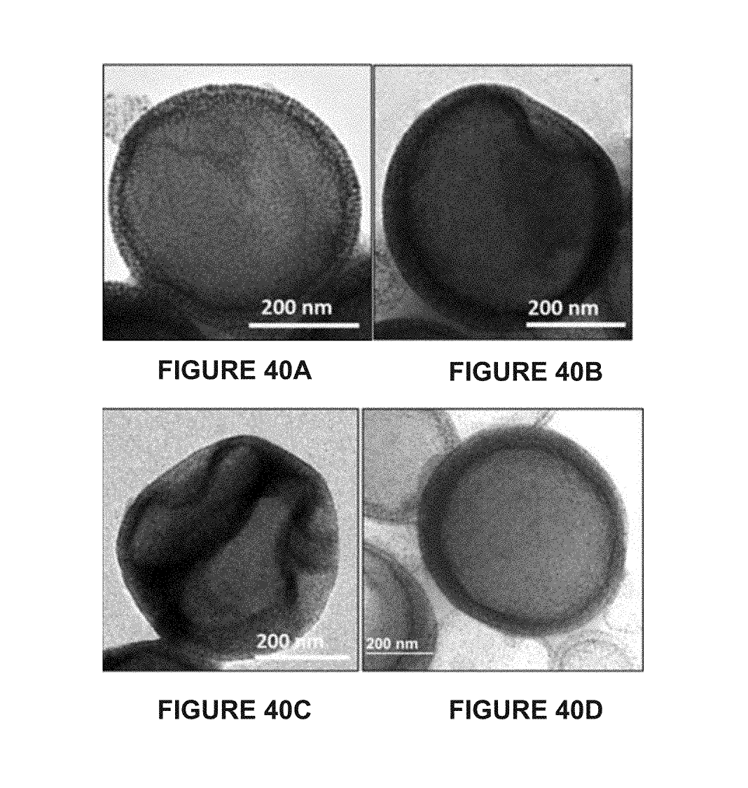

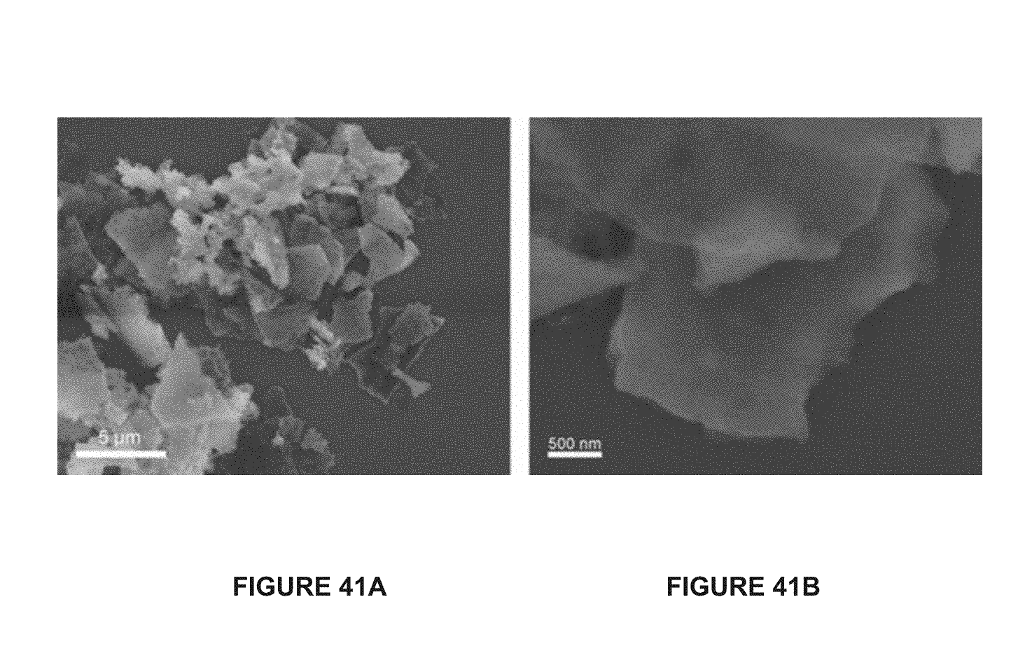

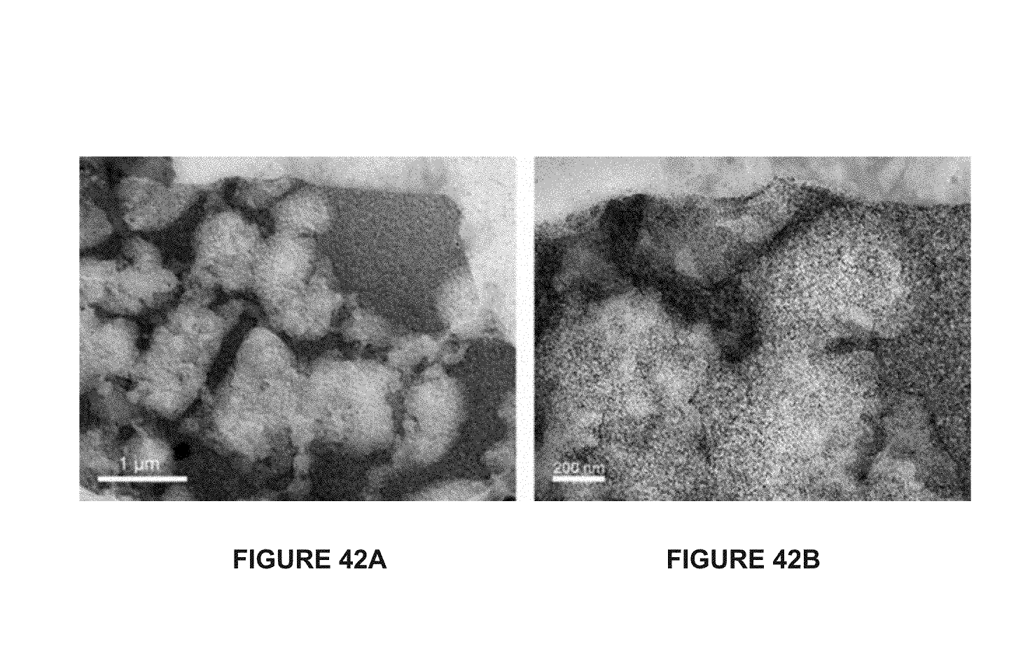

The formation of MVs with tunable morphology and membrane thickness was determined by the relative weight content of PS-b-PAA to SPIONs (W_{BCP}/W_{SPION}) in the assembly process. TEM images in FIGS. 34A, 34B, 35A, and 35B show the MVs with different membrane thickness obtained by varying W_{BCP}/W_{SPION} (at fixed W_{SPION} of 100 μg) for assembly. Without the addition of PS-b-PAA, pristine PS-b-PEO-tethered SPIONs assembled into MoMVs with a monolayer of SPIONs (FIG. 34A). This is supported by the analysis of membrane thickness: the average wall thickness (T_{MV}) of the MoMVs was measured to be 9.8±1.5 nm, which is close to the size of SPIONs (9.2±0.6 nm) (FIG. 36A). When W_{BCP}/W_{SPION}≈0.8, DoMVs were obtained with two layers of SPIONs embedded in the polymer wall (FIG. 34B). In this case, the measured T_{MV} of 24.1±3.8 nm was slightly larger than two times of T_{MV}(19.6 nm) of the monolayer membrane, because of the presence of additional PS-b-PAA (FIG. 36A). Further increasing W_{BCP}/W_{SPION} to the range of ˜1.6-3.2 resulted in the formation of MuMVs with more layers of SPIONs (FIGS. 35A and 35B). Meanwhile, T_{MV} of the MVs increased up to 93.2±12.9 nm for MuMVs with the thickest membrane (FIG. 36B). However, at W_{BCP}/W_{SPION}>3.2, aggregates rather than vesicles were obtained. The co-assembly of different structures was summarized in a product diagram in FIG. 36B. With increasing amount of PS-b-PAA, the morphology of assemblies underwent a transition from MoMVs, DoMVs, to MuMVs, and eventually to random aggregates (FIGS. 37A, 37B, 38A, 38b, 39A, 39B, 40A-40D, 41A, 41B, 42A, and 42B). At a fixed W_{BCP}, a morphological transition from aggregates to MuMVs or from MuMVs to DoMVs was observed with increasing W_{SPION}, depending on the value of W_{BCP}.

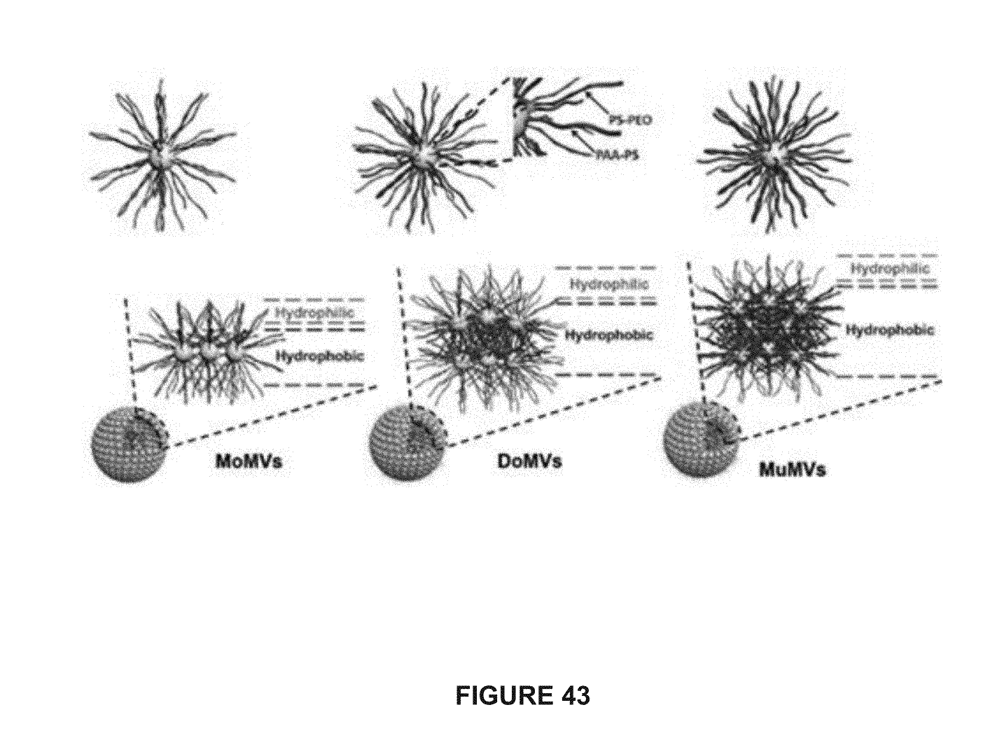

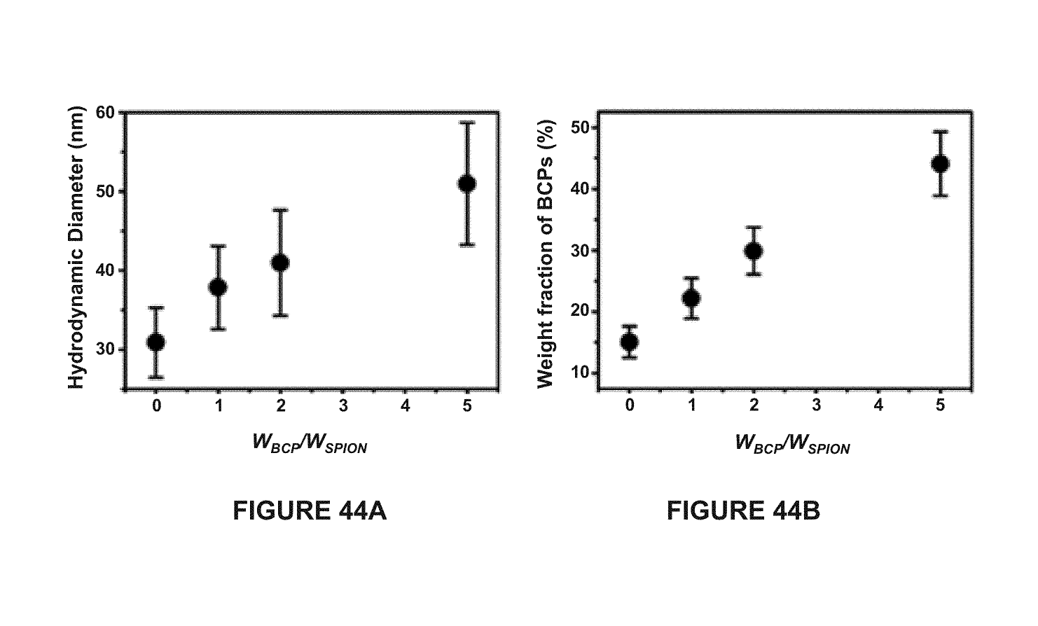

The assembly of MVs with controlled membrane thickness is believed to be attributed to the modulation of the physical property of colloidal building blocks via the cooperative interactions between PS-b-PEO grafted SPIONs and free PS-b-PAA. FIGS. 43, 44A, and 44B illustrate the hypothetical mechanism of morphological control in the assembly. In the absence of free PS-b-PAA, the long, flexible PS-b-PEO chains grafted on the NPs undergo conformation change in response to polar solvent water. Hydrophilic PEO blocks are preferentially exposed to water while hydrophobic PS blocks tend to be shielded from water to minimize the interfacial free energy, thus leading to the formation of MVs composed of a monolayer of SPIONs (FIG. 43). When PS-b-PAA is added in the dispersion of PS-b-PEO grafted SPIONs in THF, the free BCPs can bind to the NPs with hydrophobic PS ends extending to the solvent media, due to the strong affinity of carboxyl groups to SPIONs. The relatively low σ of PS-b-PEO on SPIONs (vs. σ≈˜0.10 chains/nm² for thiol-terminated BCPs on Au NPs of similar size) may also contribute to the insertion of PS-b-PAA in-between PS-b-PEO brushes on the surface of SPIONs. Upon the rehydration of dried thin films of such mixture in water, the hydrophobic PS ends of inserted PS-b-PAA chains tend to segregate away from the non-solvent, while maximizing the exposure of hydrophilic PEO segments of PS-b-PEO brushes. At optimal ratio of W_{BCP}/W_{SPION}, DoMVs with bilayer of SPIONs are formed after assembly. Further increasing the amount of PS-b-PAA leads to an even higher σ of PS blocks on NP surface and the further increase in the hydrophobicity of the NP building blocks. As a result, more SPIONs grafted with both PS-b-PAA and PS-b-PEOsegregated in the center of the vesicular membrane, leading to the formation of MuMVs with more layers of SPIONs.

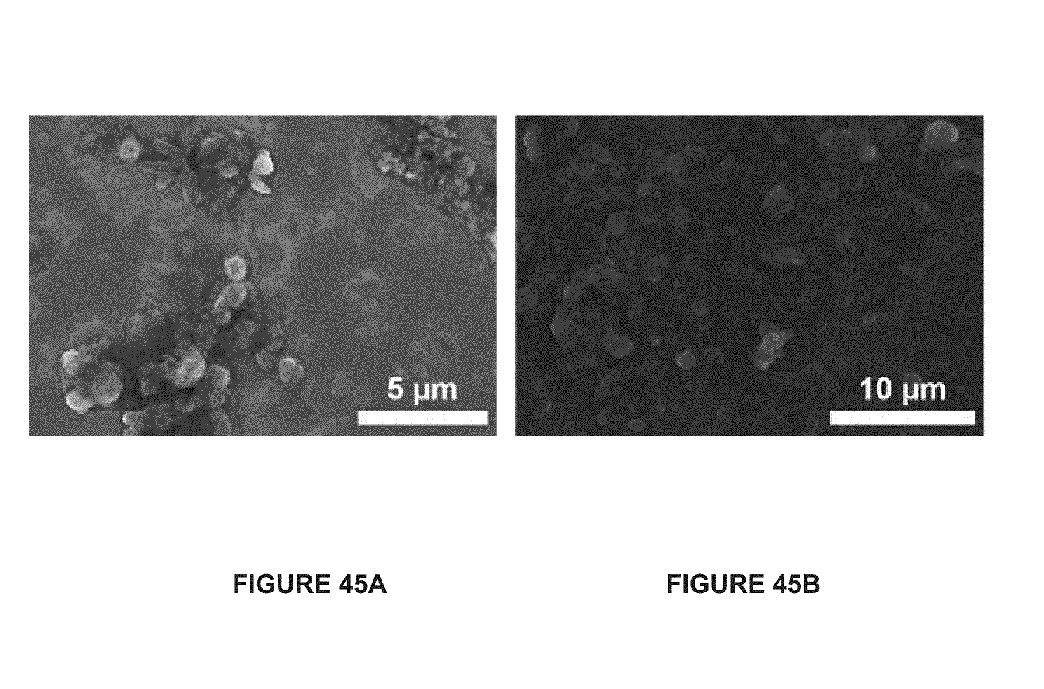

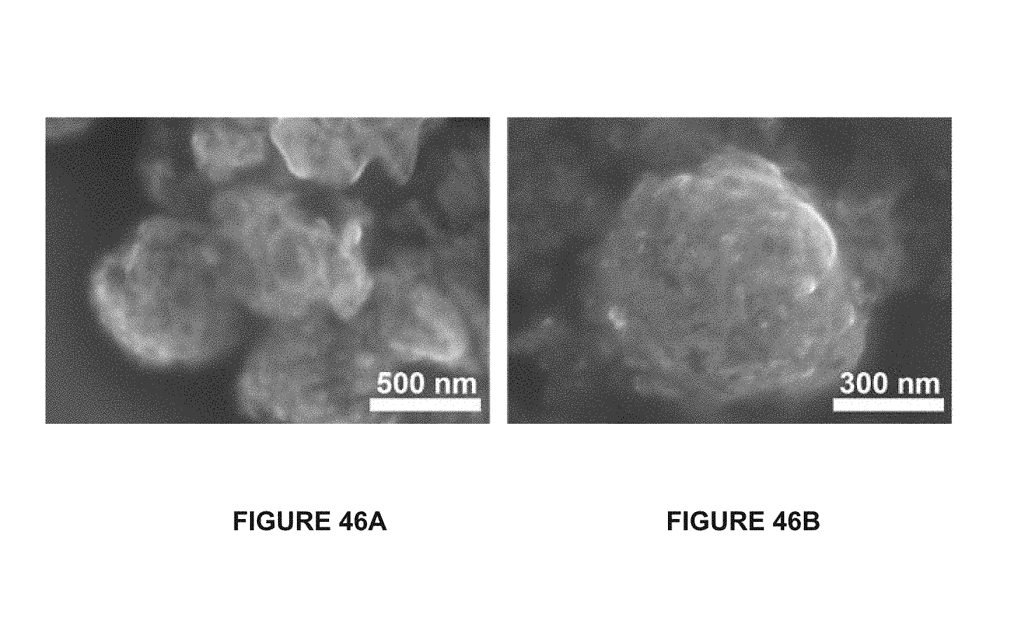

This proposed mechanism is supported by a control experiment with free PS-b-PEO that provides evidence on the attachment of PS-b-PAA on the PS-b-PEO grafted SPIONs. First, when free PS-b-PEO instead of PS-b-PAA was added, the assembly of BCP-SPIONs led to the formation of irregular aggregates rather than MVs with controlled layers of SPIONs in membranes (FIGS. 45A, 45B, 46A, and 46B). Second, the hydrodynamic diameter of BCP-SPIONs was found to increase significantly from 30.87±4.44 nm to 50.97±7.75 nm with increasing feeding ratio of PS-b-PAA, as shown in FIG. 44A (BCP-SPIONs were dispersed in THF for DLS analysis and untethered BCPs were removed by careful centrifugation). This could be attributed to a denser polymer layer around SPIONs formed by anchoring PS-b-PAA onto NP surface. The same trend was also observed in the thermogravimetric analysis (TGA) of the amount of ligands on SPIONs with the addition of PS-b-PAA (un-attached PS-b-PAA was removed by centrifugation). The weight fraction of polymers increased from 15.1% for pristine PS-b-PEO-tethered SPIONs to 44.0% for BCP-SPIONs when excess PS-b-PAA was added (W_{BCP}/W_{SPION}=5) (FIG. 44B).

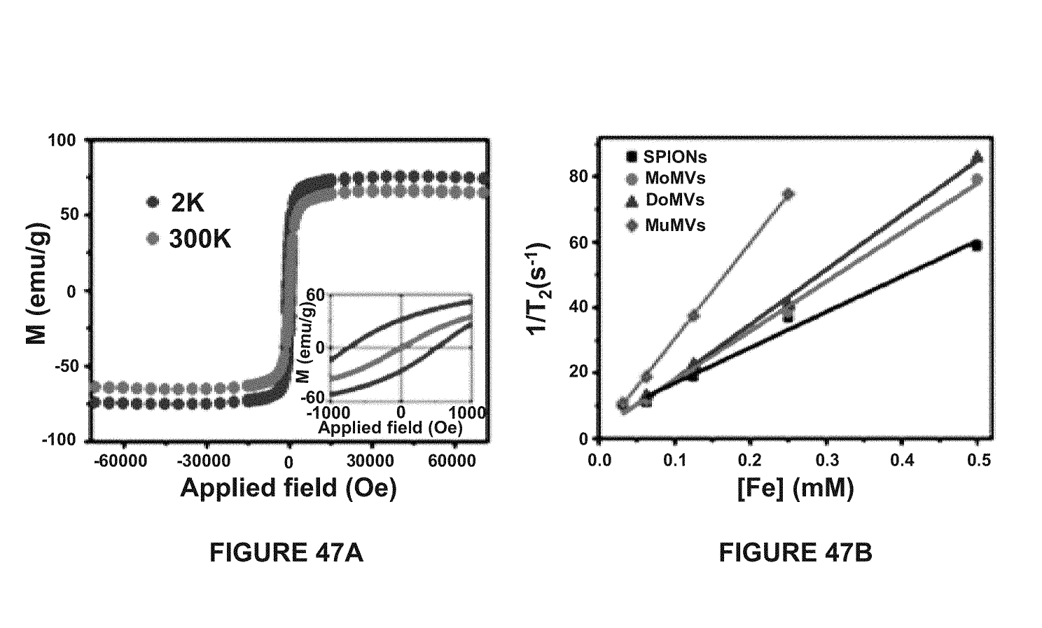

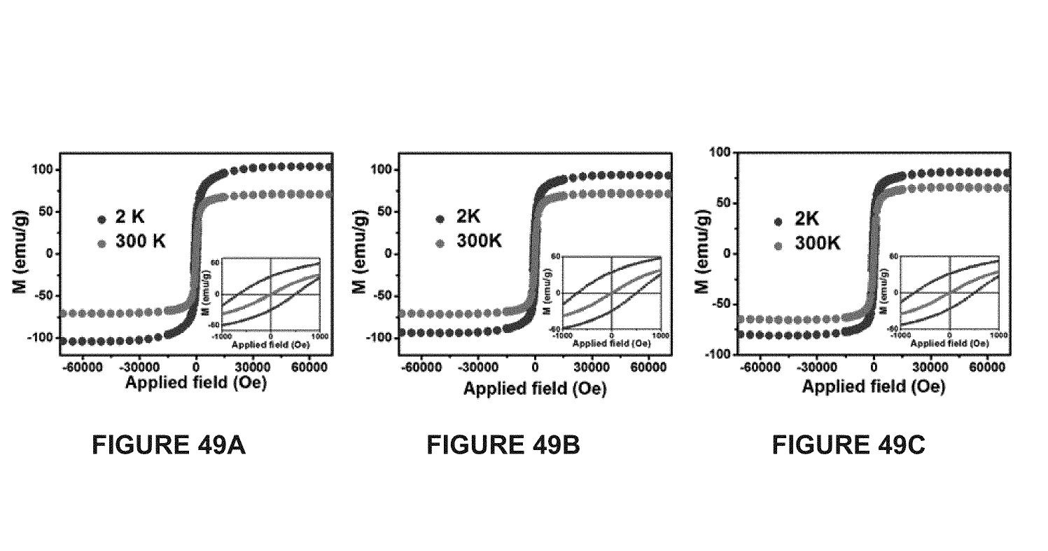

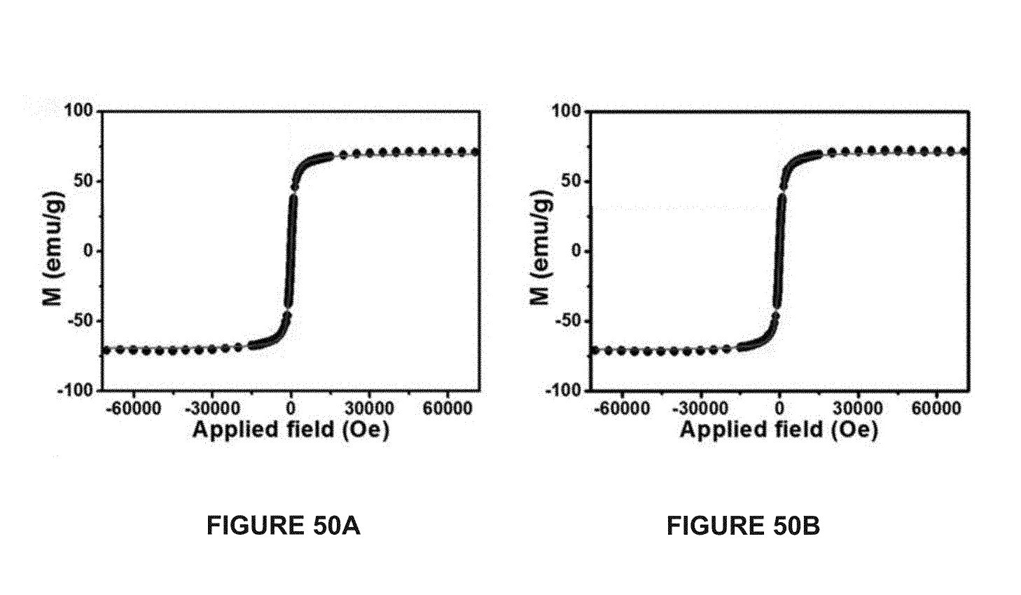

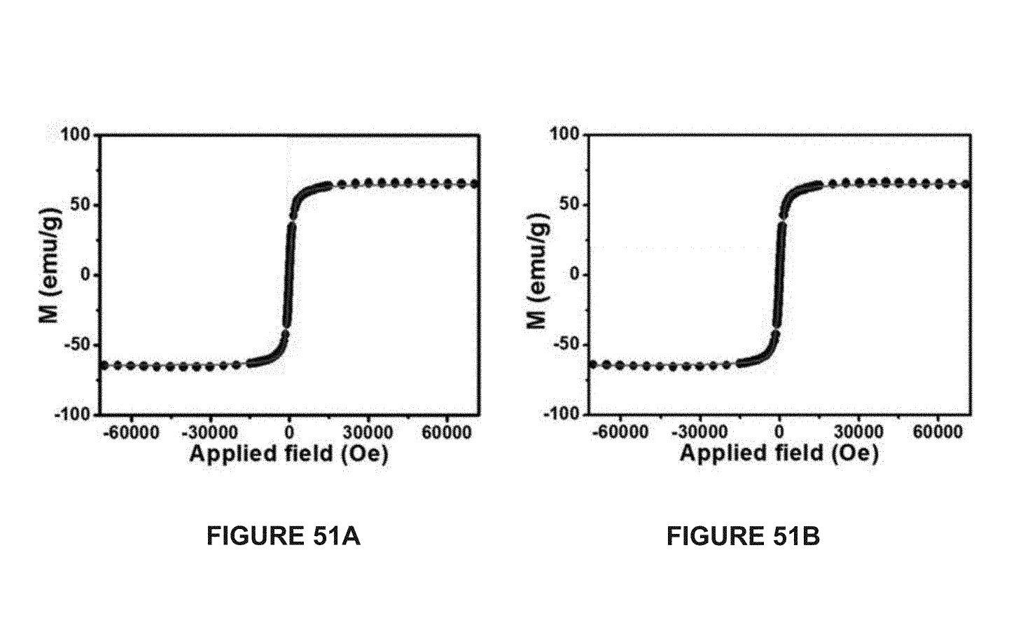

The MVs exhibited superparamagnetic properties at room temperature, although their overall diameter was well above the threshold size for the superparamagnetic/ferromagnetic transition of iron oxide NPs. As shown in the superconducting quantum interference device (SQUID) measurement (FIGS. 47A and 49A-49C), the hysteresis loop of MVs showed no remanence at 300 K, indicating their superparamagnetic behavior similar to that of individual SPIONs. By fitting the data from SQUID tests with the Langevin paramagnetic function, the magnetic moments for individual SPIONs, MoMVs, DoMVs, and MuMVs were estimated to be 8.28×10⁻¹⁷ emu/particle, 7.79×10⁻¹⁴ emu/vesicle, 1.66×10⁻¹³ emu/vesicle and 6.98×10⁻¹³ emu/vesicle, respectively (FIGS. 47B, 50A, 50B, 51A, and 51B). This suggests that individual MuMVs can respond more strongly to magnetic field than individual SPIONs and other assemblies. When a magnet (3.8×3.8×2.5 cm, 0.43 T) was applied, MuMVs were completely moved from solution towards the magnet within 2 minutes, while SPIONs remained homogeneous in the solution without any visible movement for hours. The strong magnetic movement of MuMVs makes them more suitable for magnetic field-assisted targeting and drug delivery.

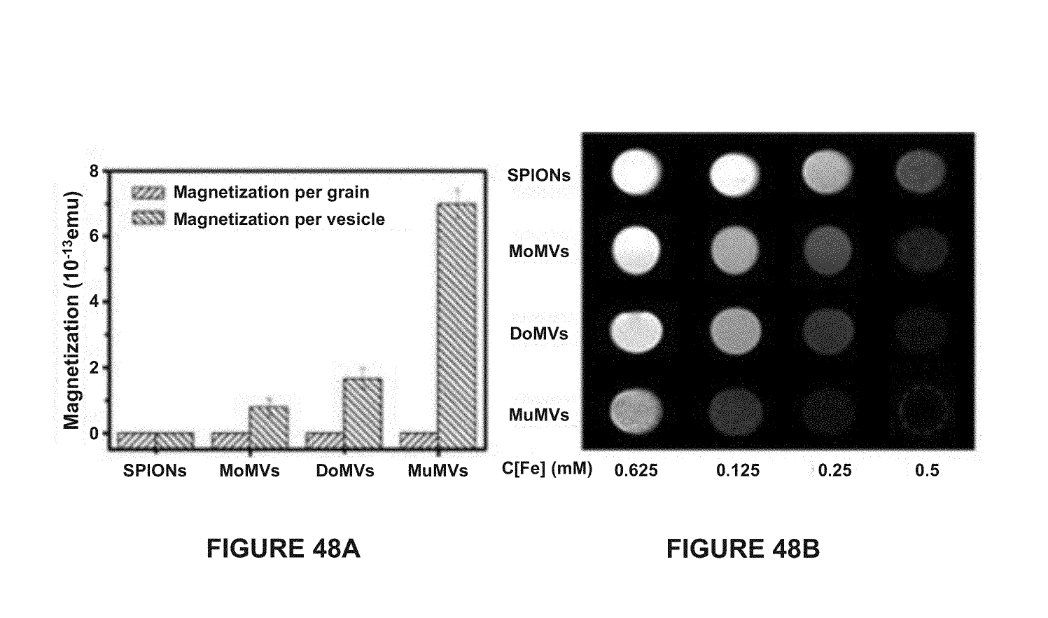

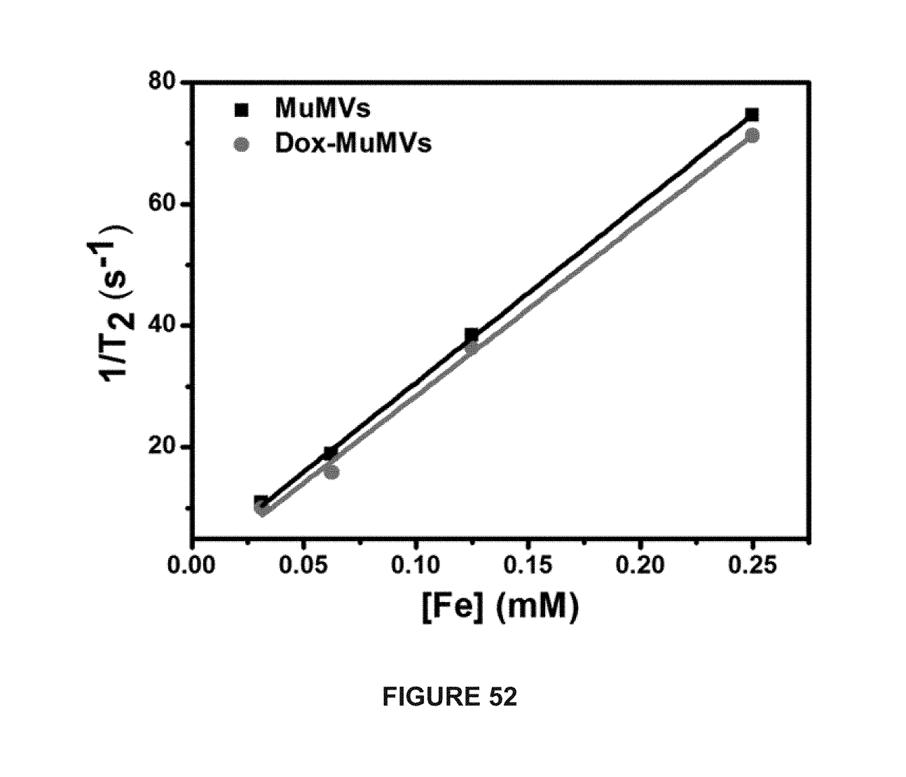

The potential use of MVs in MRI was evaluated by comparing the r₂ values of individual SPIONs, MoMVs, DoMVs, and MuMVs by plotting the inverse relaxation times (1/T₂) as a function of iron concentration [Fe] (FIG. 48A). The r₂ value determined by the slope of the plot was 293.6 mM⁻¹s⁻¹ for MuMVs, which was 1.8, 2.0 and 2.7 times higher than that for DoMVs (167.1 mM⁻¹s⁻¹), MoMVs (149.9 mM⁻¹s⁻¹) and individual SPIONs (108.7 mM⁻¹s⁻¹). The high density of SPIONs in the vesicular membranes was presumed to increase the r₂ of MuMVs due to enhanced overall magnetic moment and magnetization. The measurements were consistent with the trend of the darkness in our T₂-weighted MR images with different iron concentrations in aqueous dispersion: MuMVs>DoMVs>MoMVs>individual SPIONs (FIG. 48B). It is worth noting that the loading of therapeutic agents within the vesicles does not significantly change the r₂ value of assemblies (FIG. 52). Thus, MuMVs, which exhibit the highest magnetization and r₂ value, were chosen for subsequent in vitro and in vivo studies.

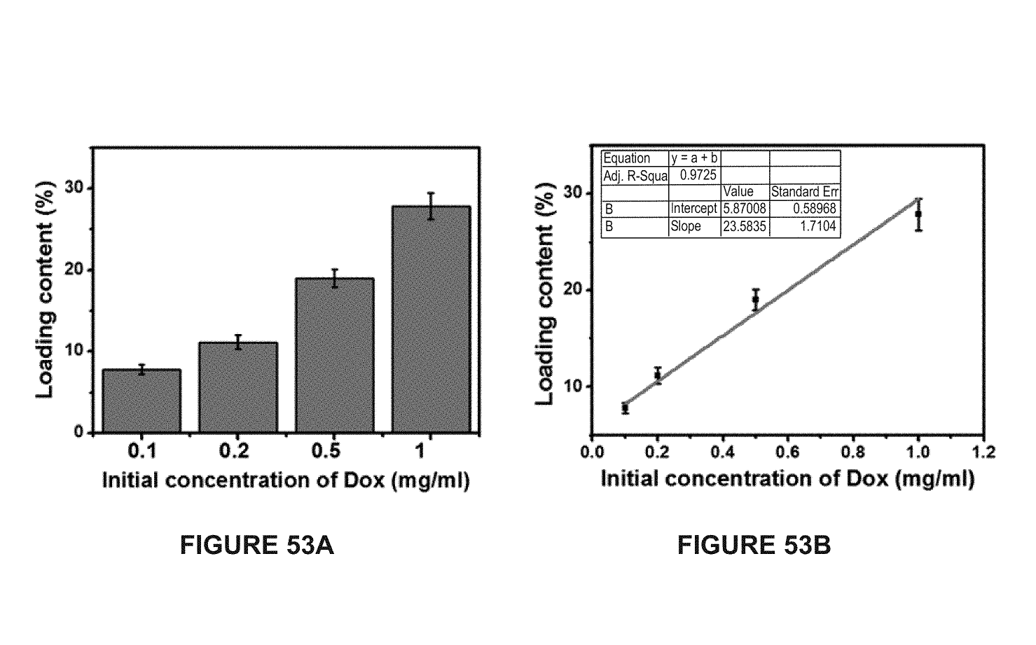

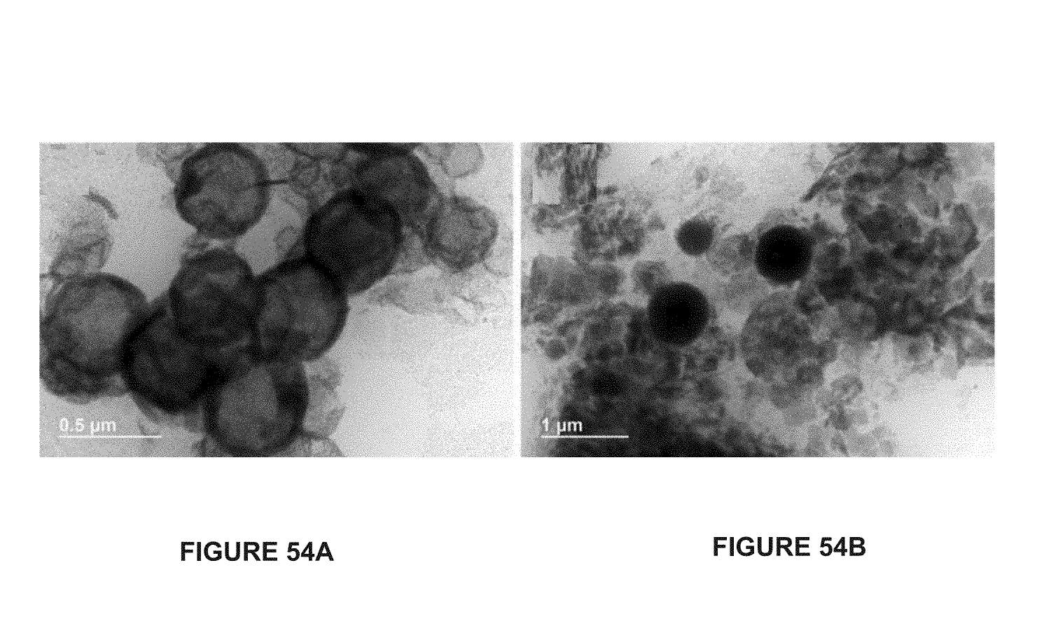

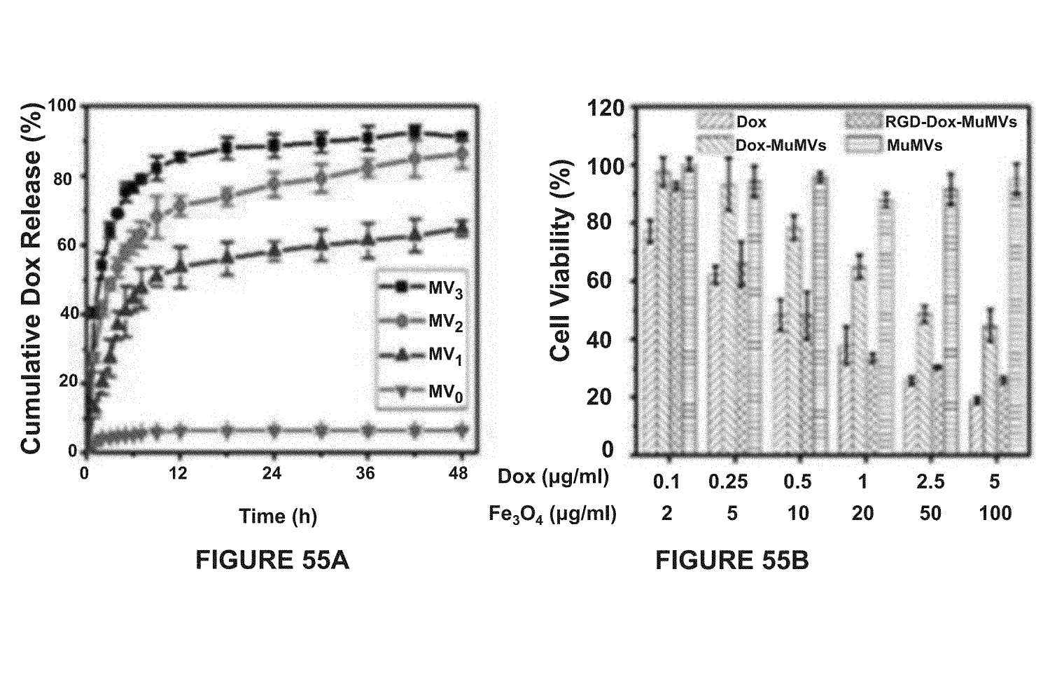

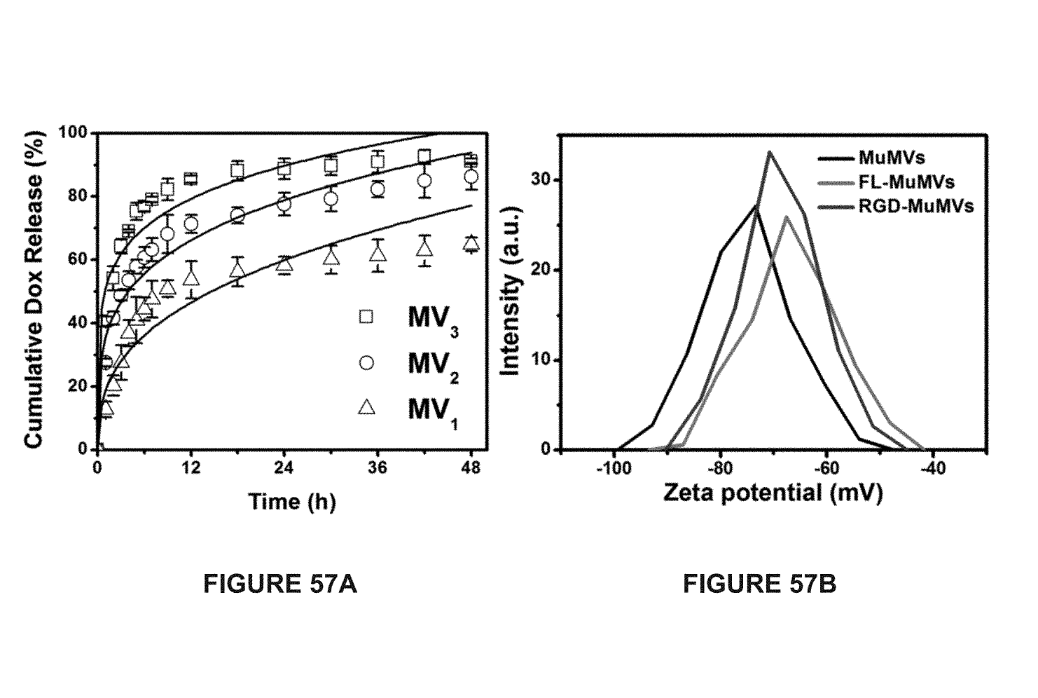

The performance of MuMVs for in vitro targeting and drug delivery to tumor cells was evaluated using Dox as a model drug. The use of film rehydration method enables more efficient encapsulation of therapeutic agents than post-encapsulation using dialysis approach. The loading capacity of Dox in MuMVs could be tuned from 7.8% to 27.8% by controlling the concentration of Dox in solutions for rehydration (FIGS. 53A and 53B). The maximum loading content of 27.8% was achieved with an initial concentration of Dox at 1 mg/mL, while further increase in the initial concentration of Dox in solutions to ˜1.5 mg/mL led to a drastic drop in the loading content of Dox in MuMVs. This was presumed to be attributable to the formation of broken vesicles due to the significantly increased viscosity of Dox solution (FIGS. 54A and 54B). The release of Dox from the MVs was found to be strongly dependent on the composition of the vesicular membrane (FIG. 55A). The release rate of Dox from MVs increased with the increasing W_{BCP}/W_{SPION}, while a negligible amount of Dox release (<7%) was observed from MoMVs after 48 hours. This can be explained by the impermeability of SPIONs to Dox and high mobility of low molecular weight PS₁₀₆-b-PAA₄. When more PS-b-PAA were added, the less dense packing of impermeable SPIONs increased the transport of Dox molecules through the membranes. Moreover, un-tethered PS-b-PAA chains with high mobility may present in the vesicular membranes, leading to the increase in the permeability of membranes for small molecular drugs. The release curve fit well to the semi-empirical Korsmeyer-Peppas model, indicating a diffusion-controlled release of Dox from Dox-loaded MuMVs (Dox-MuMVs) (FIG. 57A).

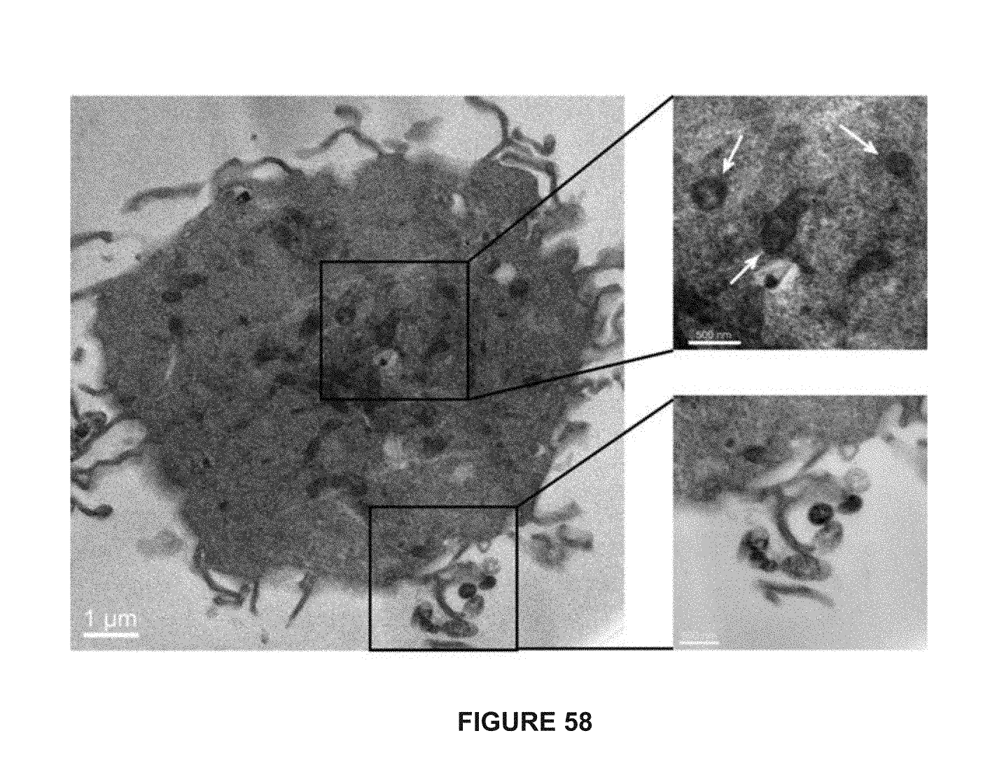

Fluoresceinamine (FL) for labeling and RGD peptides for targeting were conjugated onto the carboxyl groups of PS-b-PAA via a carbodiimide reaction. The resultant PS-b-PAA was used to co-assemble with BCP-tethered SPIONs to form surface-functionalized MuMVs. The zeta potential of PS-b-PAA increased slightly after conjugation due to the consumption of negatively charged carboxyl groups (FIG. 57B). Subsequently, the FL and RGD conjugated Dox-MuMVs (FL-RGD-MuMVs) were incubated with U87MG human malignant glioma cells for 1 hours, followed by confocal laser scanning microscopy (CLSM) imaging. Cells treated with saline or FL-labelled Dox-MuMVs without RGD modification (FL-MuMVs) were used as control groups. FIG. 55B shows that considerable amount of FL-RGD-MuMVs were internalized and distributed in the cytoplasm of U87MG cells with the overexpression of αᵥβ₃ integrin that specifically binds to RGD sequence. In contrast, the internalization of FL-MuMVs was much lower, as evidenced by a weaker green and red fluorescence inside the tumor cells. The cellular internalization of FL-RGD-MuMVs was further confirmed by TEM analysis, where numerous vesicles were found in the cytoplasm of the tumor cell (FIG. 58). Minimal green fluorescence was observed inside the nucleus as the MuMVs are too large to penetrate nuclear pores. However, large amount of Dox could be released from the vesicles and further diffuse into the nucleus to inhibit tumor growth after cellular internalization, as evidenced by fairly strong red fluorescence throughout the cells.

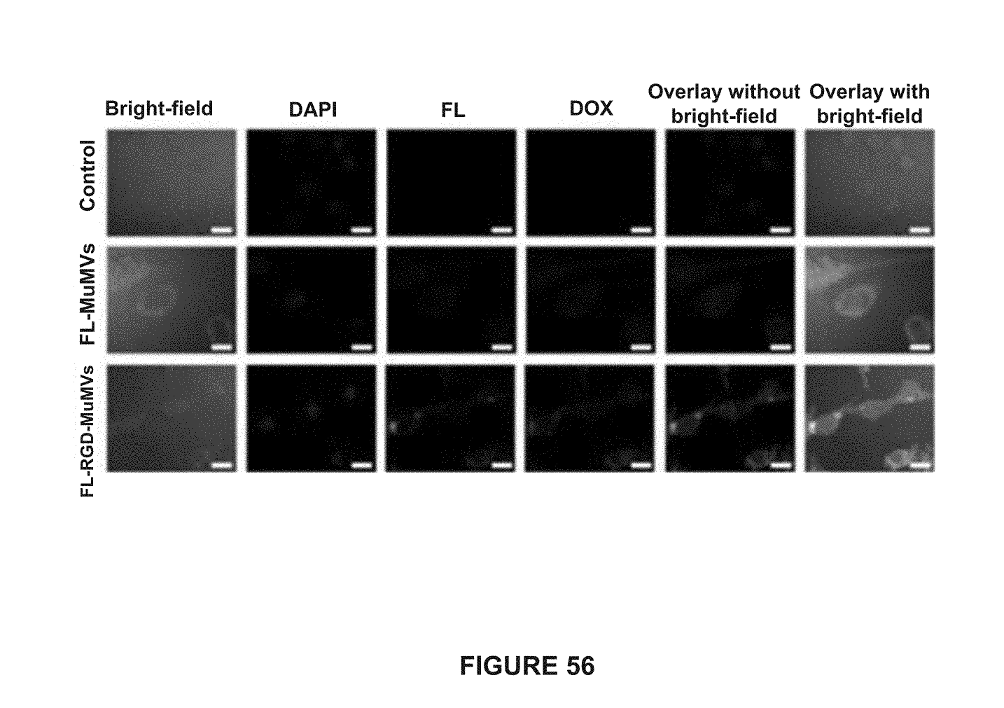

The in vitro cytotoxicity of free Dox, MuMVs, Dox-MuMVs, and RGD-Dox-MuMVs against U87MG cells was evaluated by using the 3-(4,5-dimethylthiazol-2-yl)-2,5-diphenyltetrazolium bromide (MTT) assay (FIG. 56). No significant toxicity was found for cells treated with MuMVs at all studied concentrations. In contrast, free Dox, Dox-MuMVs, and RGD-Dox-MuMVs all exhibited a dose-dependent cytotoxicity on the tumor cells with IC₅₀ values of 470, 2381 and 474 ng/mL (Dox concentration), respectively. This suggests a comparable tumor inhibition efficacy of RGD-Dox-MuMVs to free Dox molecules, both of which could be efficiently internalized into tumor cells. Conversely, a much lower tumor inhibition by Dox-MuMVs could be attributed to the limited diffusion of Dox from non-internalized vesicles into tumor cells, since they were not able to effectively enter tumor cells within the incubation period.

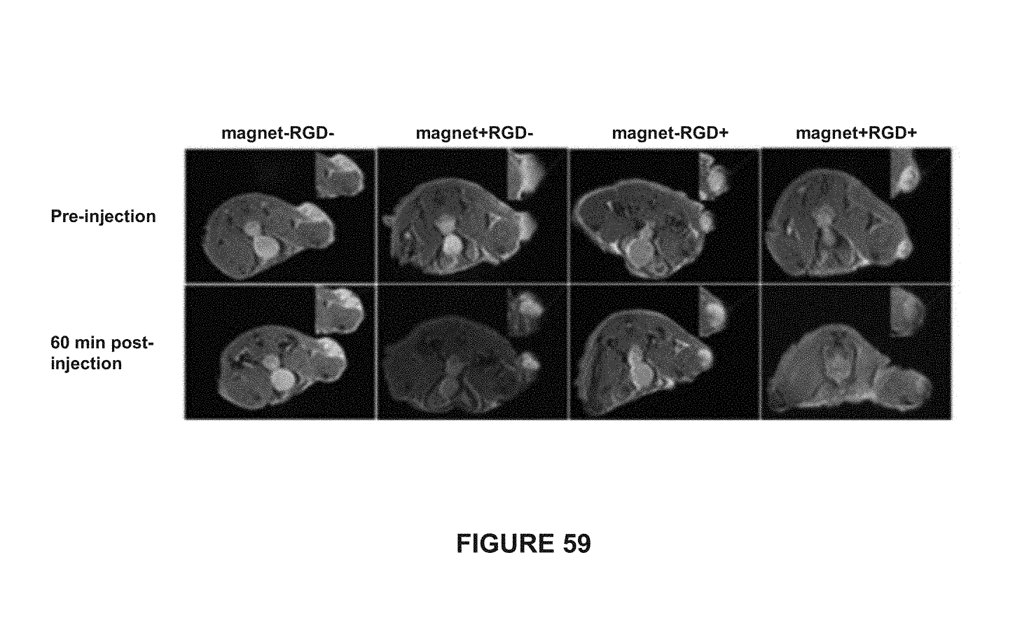

The synergistic magnetic field-driven targeting and RGD-based active targeting of tumors was assessed in athymic nude mice bearing U87MG tumors. The mice were intravenously injected with one of the groups: Dox-MuMVs (magnet+/−) or RGD-Dox-MuMVs (magnet+/−) at equivalent Dox dose (5 mg Dox/kg corresponding to a 65 mg Fe₃O₄/kg). Subsequently, a magnetic field (0.43 T) was applied for 1 hour for the positive (+) groups. Compared with mice injected with Dox-MuMVs (magnet−), the enhancement in the negative MRI contrast (darkening) in tumors was found to be 20.1%, 54.6%, and 87.6% from the baseline for the groups of RGD-Dox-MuMVs (magnet−), Dox-MuMVs (magnet+), and RGD-Dox-MuMVs (magnet+), respectively (FIG. 59). The result confirms that intravenously-injected MuMVs can be effectively enriched in tumors due to the synergistic effect of magnetic and active tumor targeting. The magnetic-field enhanced accumulation of MuMVs is more significant than individual SPIONs and even micelles or clusters composed of SPIONs.

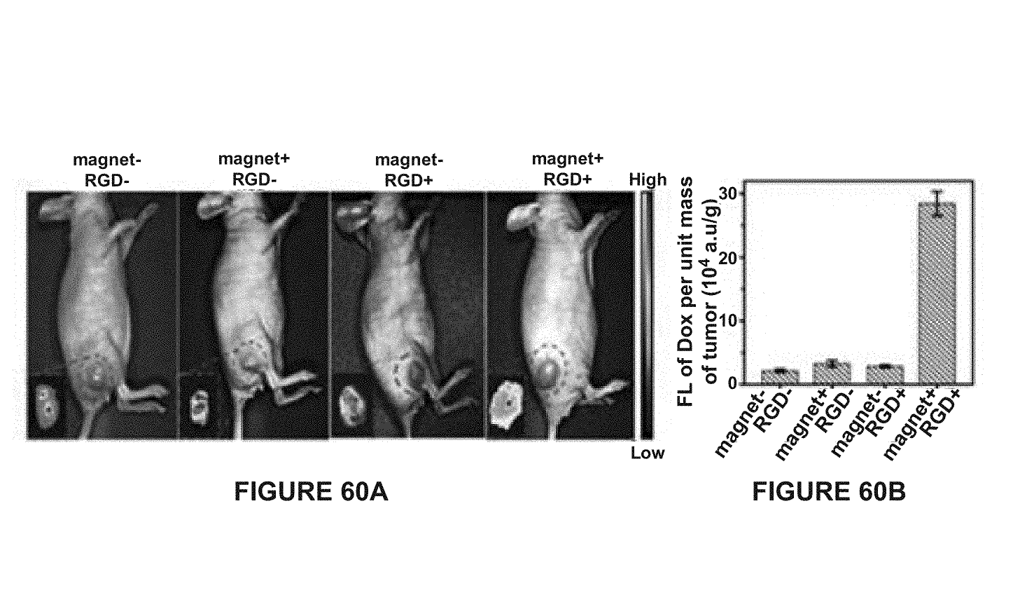

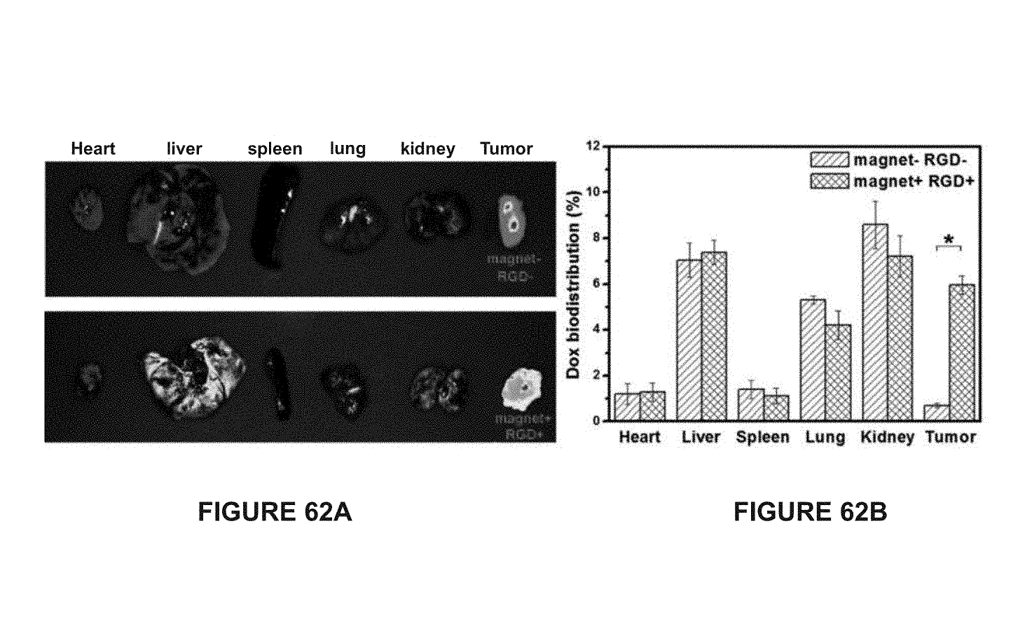

The delivery of Dox in tumors for the aforementioned groups was evaluated by tracing the red fluorescence of Dox. Only a weak fluorescence signal in tumors was observed for mice treated with Dox-MuMVs (magnet−) and RGD-Dox-MuMVs (magnet−) (FIG. 60A). The fluorescence was slightly higher for mice treated with Dox-MuMVs (magnet+). In contrast, the group of RGD-Dox-MuMVs (magnet+) exhibited the strongest fluorescence of Dox in tumors among all the groups. After in vivo imaging, the mice were sacrificed and tumor tissues were harvested for quantitative ex vivo imaging. Compared with the mice injected with Dox-MuMVs (magnet−), the fluorescence in tumor tissues exhibited a 1.6-, 1.3-, and 11.8-fold increase for the groups of Dox-MuMVs (magnet+), RGD-Dox-MuMVs (magnet−), and RGD-Dox-MuMVs (magnet+), respectively, indicating the enhanced delivery efficacy thanks to a synergetic magnetic and active targeting strategy (FIG. 60B). Major organs of mice were also collected for ex vivo quantitative analysis of Dox biodistribution with and without targeting strategies (FIGS. 62A and 62B). In the control groups (magnet-, RGD−), only 0.70% of injected Dox was observed at the tumor site while 7.0% and 1.4% of Dox was found in liver and spleen, respectively. However, the accumulation of Dox in tumor significantly increased to ˜6.0% with combined magnetic and active targeting, comparable to those in liver (7.4%) and spleen (1.1%).

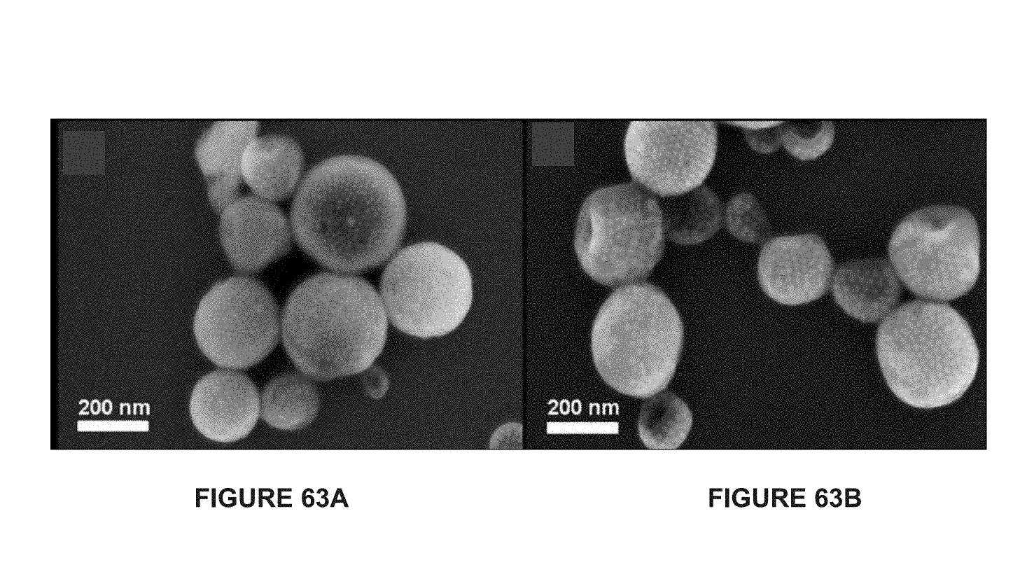

The rapid clearance of relatively large particles by the reticuloendothelial system (RES) is known to reduce the accumulation of particles in diseased sites. It is interesting that the fast accumulation of MuMVs in tumors via combination targeting strategies ensures less RES capture and enhanced delivery efficiency, although their size is larger than 200 nm. This can be attributed to the following two aspects. First, the MuMVs are composed of highly elastic vesicular membrane (in contrast to rigid solid NPs), which enables them to deform their shape and to penetrate into tumor tissues under an external magnetic field. This is partially confirmed by the fact that the MuMVs with a diameter of ˜260 nm can readily pass through channels with a diameter of 200 nm (FIGS. 63A and 63b). Second, the magnetic force exerted on a single MuMV is directly proportional to the cumulative SPIONs in a vesicle. With the increase of vesicle size, more SPIONs can be loaded in the vesicle membrane and a stronger net magnetic force could be exerted to drive MuMVs to accumulate in tumors. The result is in agreement with previous reports that SPIONs-loaded magnetic capsules larger than 200 nm performed better in magnetic targeting than small-sized SPIONs in vivo.

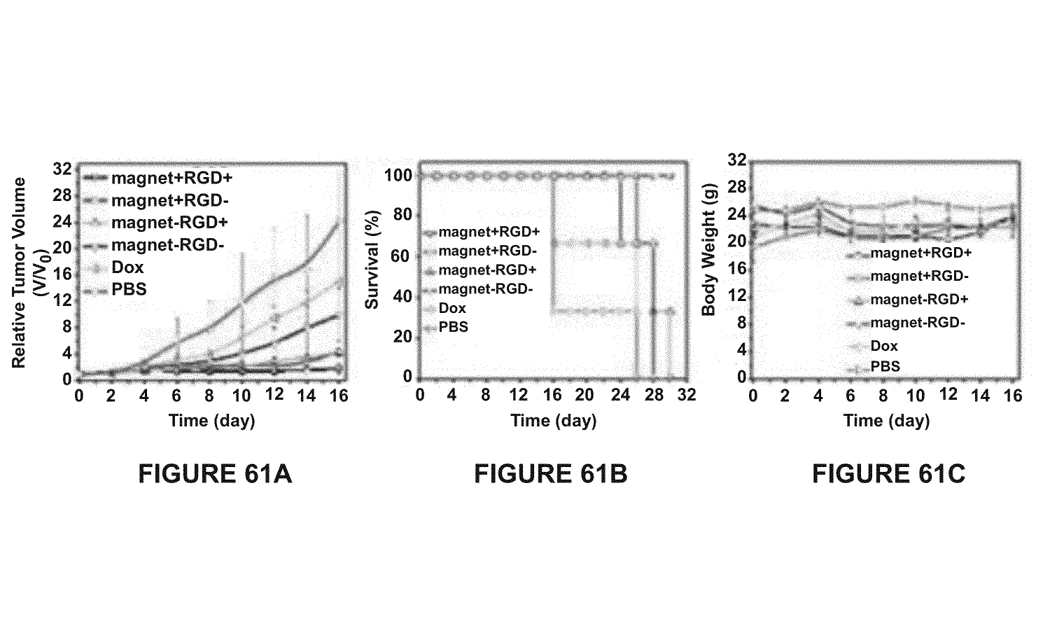

The therapeutic efficacy was evaluated by monitoring the tumor volume change every two days over 30 days (FIG. 61A). It was found that the mice treated with PBS buffer exhibited a rapid increase in the size of the tumors. Minor delay in tumor growth was observed in the mice treated with Dox or Dox-MuMVs (magnet−) due to the low delivery efficiency. Thanks to the active or magnetic targeting capacity, both RGD-Dox-MuMVs (magnet−) and Dox-MuMVs (magnet+) treated mice exhibited improved efficacy of tumor growth inhibition. In contrast, the tumor was nearly completely eradicated for the mice treated with RGD-Dox-MuMVs (magnet+). Moreover, the mice treated with RGD-Dox-MuMVs (magnet+) exhibited a much longer survival life without a single death or tumor reccurrence (over 30 days) as compared to all the other groups (FIG. 61B). Meanwhile, negligible loss of body weight was observed for all the groups of mice during the therapeutic period (FIG. 61C), indicating minimal systemic toxicity of drug carriers.

A new class of MVs with tunable layers of densely packed SPIONs in the polymeric membrane for tumor-targeted imaging and delivery was developed. The morphology of the vesicles could be controlled from monolayer, double layer to multilayer vesicles and the membrane thickness increased significantly with increasing feeding ratio of PS-b-PAA to SPIONs. The MuMVs with a thicker membrane and higher SPIONs density were found to possess unique features such as enhanced contrast in MM, high magnetization per vesicle, and tunable release profile of therapeutic agents. Upon intravenous administration, the MuMVs conjugated with RGD targeting moieties can be efficiently enriched at the tumor site in vivo with the assistance of an external magnetic field, thanks to the synergistic magnetic and active tumor targeting effect. The enhanced tumor accumulation of MuMVs enables the efficient imaging of tumors by MM, tumor targeted delivery of payload and a resultant enhanced tumor inhibition.

### Example 13

### Materials for Examples 14-27

Dopamine hydrochloride, 6-maleimidohexanoic acid N-hydroxysuccinimideester, triethylamine (>99.5%, TEA), styrene, azobis(isobutyronitrile) (AIBN), 4-cyano-4-(phenylcarbonothioylthio) pentanoic acid (CPPA), dioxane, tetrahydrofuran (THF), N,N-dimethylformamide (DMF), n-butylamine, N-hydroxysuccinimide (98%, NHS), N-(3-dimethylaminopropyl)-N′-ethyl-carbodiimide hydrochloride (98%, EDC), oleic acid (99%), 1-octadecene, doxorubicin hydrochloride (98.0-102.0%), Dulbecco's modified Eagle's medium (DMEM), 2-(4-amidinophenyl)-6-indolecarbamidine dihydrochloride (DAPI dihydrochloride), and fluoresceinamine isomer I were purchased from Sigma-Aldrich. Polystyrene-b-poly(acrylic acid) (PS₁₀₆-b-PAA₄) was purchased from Polymer Source Inc. Fetal bovine serum (FBS), DPBS, trypsin-EDTA and penicillin/streptomycin (5000 U/mL) were purchased from Thermo Fisher Scientific.

### Example 14

### Synthesis of Maleimide-Terminated Dopamine

Maleimide-terminated dopamine was synthesized by the carbodiimide reaction following the procedure of Mazur, M., et al., Nanoscale 5:2692-2702 (2013). Dopamine hydrochloride (1.92 g, 0.010 mol) and triethylamine (1.28 g, 0.013 mol) were dissolved in anhydrous methanol (10 mL) and then added dropwise to a solution of 6-maleimidohexanoic acid N-hydroxysuccinimide ester (2.6 g, 0.008 mol) in anhydrous CH₂Cl₂ (100 mL). The mixture was stirred vigorously for 48 hours under nitrogen and washed three times with HCl (0.5 M, 80 mL). The solvent was evaporated and the crude product was purified by column chromatography (SiO₂/CH₂Cl₂/MeOH 10:1).

### Example 15

### Synthesis of Thiol-Terminated BCPs.

Thiol-terminated block copolymers (BCPs) of HS-PS₂₆₀-b-PEO₄₅ were synthesized by reversible addition-fragmentation chain transfer polymerization following the procedure described in He, J., et al., J. Am. Chem. Soc. 134:11342-11345 (2012). Styrene, chain transfer agent (PEO-CTA), and AIBN were dissolved in dioxane with a molar ratio of 300:1:0.2. The solution was filled with nitrogen and then put into a pre-heated oil bath at 85° C. for 20 hours. The product was precipitated in hexane and dissolved in THF to remove unreacted monomers and impurities. Molecular weight of the BCPs characterized by ¹H NMR was 29.0 kg/mol, by comparing the integrals of the resonance peaks of aromatic ring of polystyrene (PS) block (6.4-7.3 ppm) and the methylene groups of PEO-CTA (3.65 ppm). The CTA-PS₂₆₀-b-PEO₄₅ was dissolved in THF with an excess of n-butylamine under nitrogen for 4 hours to convert CPPA into thiol groups. The resulting SH-PS₂₆₀-b-PEO₄₅ was obtained by precipitation in hexane twice and dried under vacuum for 24 hours.

### Example 16

### Synthesis of Dopamine-Terminated BCPs.

Dopamine-terminated block copolymers (BCPs) were synthesized by reacting maleimide-terminated dopamine with thiol-terminated BCPs through a Michael addition reaction. Maleimide-terminated dopamine was first synthesized by the carbodiimide reaction following the procedure of Mazur, M., et al., Nanoscale 5:2692-2702 (2013). Maleimide-terminated dopamine (346 mg, 1 mmol) and thiol-terminated BCPs (2.9 g, 0.1 mmol) were then dissolved in 10 mL DMF and the mixture was stirred under nitrogen for 120 hours. The dopamine-terminated BCPs were obtained by precipitating in water/ethanol mixture (1/3 by volume) for three times and dried under vacuum for 24 hours. The synthesized polymers were dissolved in CDCl₃ and characterized by ¹HNMR.

### Example 17

### Synthesis of SPIONs.

Hydrophobic superparamagnetic iron oxide nanoparticles (SPIONs) were prepared via thermal decomposition of iron-oleate complex by using oleic acid as the stabilizing agent following the procedure of Park, J., et al., Nat. Mater. 3:891-895 (2004). Iron-oleate complex (3.6 g, 4 mmol) and oleic acid (0.57 g, 2 mmol) were dissolved in 1-octadecene (20 g) at room temperature. The mixture was heated to 300° C. with a constant heating rate and then kept at this temperature for 30 minutes. The resulting solution containing SPIONs was then cooled to room temperature and washed with ethanol for three times. The precipitated SPIONs were dispersed in THF to form a stable colloidal solution with a concentration of 5 mg/mL.

### Example 18

Surface Modification and Self-Assembly of SPIONs into Magneto-Vesicles

Surface modification of SPIONs. SPIONs were modified with amphiphilic block copolymers via the chelation of dopamine with the surface of SPIONs. SPIONs (5 mg) and dopamine-terminated BCPs (15 mg) were dispersed in THF (5 mL) and the mixture was incubated for 48 hours. The solvent was evaporated and the SPIONs were washed with DMF for 5 times to remove excess BCPs. The purified BCP-tethered SPIONs were dispersed in THF with a concentration of 0.2 mg/mL. Thermogravimetric analysis (TGA) was performed to estimate grafting density of BCPs on SPIONs. The sample (5 mg) was dried and loaded into a platinum pan which was heated to 720° C. at a constant heating rate of 25° C./min under argon. The BCPs grafting density (C) was calculated using the formula:

$$σ = \frac{f ⋆ {N}_{A} ⋆ ρ ⋆ d}{6 ⋆ {M}_{n} ⋆ ( 1 - f )}$$

Here f refers to the weight fraction of the organic ligands determined by TGA analysis; N_{A} is the Avogadro constant; ρ is the bulk density of SPIONs (5.15 g/cm³); d is the average diameter of SPIONs and Mₙ is the number-average molecular weight of the PEO-b-PS. It is assumed that the density of the SPIONs is identical to the density of the bulk material and no free polymer is present.

Self-assembly of SPIONs. BCP-tethered SPIONs were assembled into magento-vesicles using the film rehydration method of Ai, X., Asian J. Pharm. Sci. 9:244 (2014). BCP-tethered SPIONs (100 μg) were mixed with PS₁₀₆-b-PAA₄ in THF. The mixture was dried to form a thin film in a glass vial under N₂ stream followed by rehydration in water or an aqueous solution of Dox under sonication for 30 seconds. The magneto-vesicles with controlled wall thickness were collected by centrifugation at 2000 rpm for 15 minutes. By adjusting the amount of PS₁₀₆-b-PAA₄ (80 μg, 160 μg, 240 μg, and 320 μg), multilayered magneto-vesicles (MuMVs) with various membrane thicknesses were achieved. Monolayer magneto-vesicles (MoMVs) were prepared in a similar way except that no PS₁₀₆-b-PAA₄ was added. For the modification of MuMVs, RGD peptides (or fluoresceinamine, FL), N,N′-dicyclohexylcarbodiimide (DCC), N-hydroxysuccinimide (NHS), and PS₁₀₆-b-PAA₄ were dissolved in dimethylformamide (DMF) with a molar ratio of 1:1.5:1.5:1, followed by mechanical stirring for 24 hours and precipitation in water/ethanol mixture (1/3 by volume) to obtain functionalized BCPs for further self-assembly. Surface-functionalized MuMVs could be obtained by using the RGD and/or FL-conjugated PS-b-PAA to assemble with BCP-tethered SPIONs.

### Example 19

### Characterizations of the Magneto-Vesicles.

The assembled magneto-vesicles were imaged using a Hitachi SU-70 Schottky field emission gun Scanning Electron Microscope (FEG-SEM) and a JEOL FEG Transmission Electron Microscope (FEG-TEM). Samples for SEM observations were prepared by dropping 5-10 μl of sample solution onto silicon wafers and dried at room temperature. TEM samples were prepared by dropping 5-10 μl of sample solution on 300 mesh copper grids covered with carbon film and dried at room temperature. To verify the vesicular structures of Mu magneto-vesicles, TEM images at different tilt angles (−60° to)60° were recorded using electron microscopic tomography. The hydrodynamic diameter of magneto-vesicles in solution was measured using a PHOTOCOR-FC light scattering instrument with a 5 mW laser of 633 nm at a scattering angle 90°. The zeta potential of magneto-vesicles in solution was measured using a SZ-100 nanoparticle analyzer. To study the mechanism of MVs formation, PEO-b-PS-SPIONs were washed with THF for 3 times after addition of PS-b-PAA and used for TGA analysis and DLS evaluation.

### Example 20

### Magnetic Properties of Magneto-Vesicles.

Magnetic property measurements were performed using a Quantum Design MPMS 3 Superconducting Quantum Interference Device (SQUID). The magnetic moment M of both MVs and individual SPIONs was measured as function of applied magnetic field H at room temperature and low temperature. The magnetic moment of an individual grain (μ) can be determined by the Langevin paramagnetic function: M(x)=Nμ(cothx−(1/x)), where x=μH/k_{B}T, N is the number of grains, H is the applied field, k_{B} is the Boltzmann's constant, and T is the absolute temperature. In this experiment, T is 300K. We let B=μ/k_{B}T and C=Nμ (B and C are constants to be determined). Fitting the data of M(x) and H into the Langevin function, two constants B and C were determined, as shown in FIG. S17. Finally the magnetic moment per grain can be simply calculated using μ=Bk_{B}T (μ_{SPIONs}=8.28E⁻¹⁷ emu; μ_{SPIONs}=8.24E⁻¹⁷ emu; μ_{DOMVs}=8.20E⁻¹⁷ emu; μ_{MuMVs}=7.87E⁻¹⁷ emu). The magnetic movement of an individual SPION is the magnetic moment of an individual grain as they are dispersed individually in an aqueous solution (M_{SPIONs}=μ_{SPIONs}8.28E⁻¹⁷ emu). However, the magnetic moment of a MV is the sum of the magnetic moment of all the subunits within the vesicle. The number of SPIONs per vesicle can be estimated according to:

$${N}_{SPIONs} = \frac{{V}_{mb} ⋆ {σ}_{mb}^{SPIONs}}{{V}_{SPIONs}}$$

where V_{mb} is the volume of membrane calculated by

$${V}_{mb} = \frac{4 π ( {R}^{3} - {r}^{3} )}{3}$$

(R is the radius of vesicle and r is the radius of cavity), σ_{mb}^{SPIONs} is the volume fraction of SPIONs inside the polymer membrane calculated by their weight ratio of SPIONs relatively to the copolymers, and V_{SPIONs} is the volume of an individual SPION calculated by

$${V}_{SPIONs} = \frac{π {D}^{3}}{6}$$

(D is the average diameter of SPIONs). Here N_{SPIONs} was calculated to be 945, 2020 and 8872 for MoMVs, DoMVs and MuMVs, respectively; and the corresponding values are M_{MoMVs}=N_{SPIONs}*μ_{MoMVs}=7.79E⁻¹⁴ emu; M_{DoMVs}=N_{SPIONs}*μ_{DoMVs}=1.66E⁻¹³ emu; M_{MuMVs}=N_{SPIONs}*μ_{MuMVs}=6. 98E ⁻¹³ emu.

### Example 21

### Magnetic Relaxivity Measurements.

The T₂ relaxivity times of individual SPIONs, MoMVs, DoMVs and MuMVs were measured at a series of different sample concentrations using a micro-MR scanner (7.0 T, Bruker, Pharmascan) with small animal-specific body coil. The Fe concentrations were determined using an Agilent 700 series inductively coupled plasma-optical emission spectrometer (ICP-OES). Briefly, a concentrated stock solution of different samples (200 μL) was added to scintillation vials. Then, 1 mL of aqua regia was added to each vial to dissolve all iron oxide nanoparticles. Finally, 9 mL of deionized water was added to the vials. The Fe concentrations of the prepared solutions were then measured using ICP-OES. The T₂ relaxivity times were plotted as a function of iron concentration to obtain the r₂ value of each sample.

### Example 22

Encapsulation and Release of Dox from MVs.

Dox-loaded MVs were prepared by rehydrating a film of BCP-tethered SPIONs in aqueous solutions of Dox, followed by centrifugation for six times to remove free drug molecules. The loading content of Dox in MuMVs (L_{Dox}) can be calculated by

$${L}_{Dox} = \frac{Mass of Dox in MuMVs}{Mass of MuMVs} ⋆ 100 \% ,$$

where mass of MuMVs was measured using ICP-OES and mass of Dox in MuMVs was evaluated using a fluorescence spectrometer. Dox solutions with predetermined concentration of Dox (from 0.1 to 2.0 mg ml⁻¹) were used for the fabrication of Dox-MuMVs (at constant MuMVs concentration of 0.2 mg ml⁻¹). For the drug release experiment, 1 ml solution of the Dox-loaded MVs was transferred to a dialysis tube with a molecular weight cutoff of 6,000-8,000 g/mol, which was incubated in a 50 ml PBS reservoir at 37° C. 1 ml solution from the reservoir was taken at scheduled time intervals and its fluorescence emission at 590 nm was measured using a fluorescence spectrometer to monitor the release of Dox from the vesicles. After each measurement, the 1 ml solution was put back into the reservoir to maintain the total volume of the buffer solution.

### Example 23

### In Vitro Cellular Uptake of MuMVs.

The human malignant glioma cell line (U87MG) were grown in Dulbecco's modified Eagle's medium (DMEM) supplemented with 10% fetal bovine serum (FBS) and 1% penicillin/streptomycin at 37° C. in a humidified 5% CO₂ atmosphere. Cells were seeded into chambered glass cover slides and grown for 24 hours. Then the culture medium was replaced with fresh medium containing saline, Dox-loaded fluoresceinamine-functionalized MuMVs (FL-MuMVs) and Dox-loaded fluoresceinamine and RGD functionalized MuMVs (FL-RGD-MuMVs), respectively (Fe concentration: 0.02 mg/mL). After incubation for 1 hour, the cells were washed three times with PBS and fixed in a 4% paraformaldehyde/PBS solution for 10 minutes. The fixative was then removed, and cells were washed again with PBS for three times and incubated with DAPI for cellular nuclei staining. The slides were washed with PBS and then observed by a confocal microscope (Zeiss LSM 710) with appropriate band-pass filters for collection of DAPI, FL, and Dox emission signals.

For TEM observations of the vesicles after cellular internalization, the FL-RGD-MuMVs were loaded into U87MG cells as described previously, except that a monolayer of cells were grown on Thermanox@ Plastic Coverslips placed inside 6-well cell-culture plates. After incubation with FL-RGD-MuMVs for 1 hour, the culture medium was replaced by the fixation solution containing 2.5% paraformaldehyde and 2.0% glutaraldehyde in 0.1 M cacodylate buffer. After 1 hour, the fixation solution was removed and samples were then washed in 0.1 M sodium cacodylate buffer for three times. The samples were dehydrated and subsequently infiltrated with Epon-Aradite for 24 hours, followed by polymerization at 60° C. for 24 hours. Ultrathin sections were cut on a Leica EM UC6 Ultramicrotome (Leica, Buffalo Grove, IL) and collected on copper slot grids for TEM observations.

### Example 24

### Cytotoxicity of Dox, MuMVs, Dox-MuMVs and RGD-Dox-MuMVs.

Cytotoxicity of Dox, MuMVs, Dox-loaded MuMVs (Dox-MuMVs) and RGD-Dox-MuMVs on the U87MG cells were evaluated using the MTT assay described in T. Mosmann, J. Immunol. Methods 65:55 (1983). Cells were plated at a density of 1×10⁴ in 96-well plates and cultured at 37° C. for 24 hours. Then the culture medium was replaced and the cells were incubated with different concentrations of Dox, Mu magento-vesicles, Dox-Mu magento-vesicles, and RGD-Dox-Mu magento-vesicles for 1 hour. The concentrations of Fe in MuMVs, Dox-MuMVs and RGD-Dox-MuMVs as well as the concentrations of Dox in free drug, Dox-MuMVs and RGD-Dox-MuMVs groups were kept constant for the purpose of comparison. Then the culture medium was replaced with fresh medium and the cells were incubated for another 12 hours, followed by the addition of 20 μL of the MTT solution (5 mg/mL). After incubation for 4 hours, culture supernatants were carefully removed and 100 μL of DMSO was added into each well to dissolve the purple precipitate. The concentration of the reduced MTT in each well was determined spectrophotometrically by subtraction of the absorbance reading at 650 nm from that measured at 570 nm using a microplate reader (SpectraMax M5). Cell viabilities were presented as the percentage of the absorbance of Dox, MuMVs, Dox-MuMVs and RGD-Dox-MuMVs treated cells to the absorbance of non-treated cells and plotted as Fe and Dox concentrations.

### Example 25

### In Vivo MRI Though Intravenous Administration.

All animal experiments were performed under a National Institutes of Health Animal Care and Use Committee (NIHACUC) approved protocol. Tumor-bearing mice were achieved by subcutaneously injecting ˜2×10⁶ U87MG cells into the right hind leg of athymic nude mice. After the tumor volume exceeded 100 mm³, MR imaging of tumor tissues was recorded as background on a high magnetic field micro-MR scanner (7.0 T, Bruker, Pharmascan) with small animal-specific body coil. Thereafter, the mice were divided randomly into four groups (5 mice in each group) and the therapeutic agents (Dox-MuMVs or RGD-Dox-MuMVs) were intravenously injected into the tumor-bearing mice at a Dox- equivalent dose of 5 mg/kg and a Fe₃O₄-equivalent dose of 65 mg/kg. An external magnetic field was applied on the experiment groups for 1 hour after injection while for the control groups no magnetic attraction was applied. Then MR images were taken to reveal the influence of magnetic attraction, RGD functionalization and synergistic magnetic and active targeting strategy on the imaging effect of the Mu magento-vesicles.

### Example 26

### In Vivo Magnetic-Guided Delivery of Dox Though Intravenous Administration.

The influence of magnetic attraction and RGD-mediated active tumor targeting on the delivery efficiency of therapeutic agents was investigated by fluorescence imaging. Briefly, Dox-MuMVs or RGD-Dox-MuMVs (5 mg Dox/kg corresponding to a 65 mg Fe₃O₄/kg) were intravenously injected into the tumor-bearing mice with or without the application of magnetic fields (5 mice in each group). Whole-animal imaging was recorded 1 hour later by using Maestro in vivo imaging system to monitor the fluorescence from Dox. Thereafter the mice were sacrificed and the tumors as well as major organs were harvested, washed and imaged to investigate the in vivo biodistribution of Dox. The fluorescence intensities from Dox per unit mass in tumor tissues were also evaluated to reflect the effects of magnetic attraction and active tumor targeting on the delivery of Dox.

### Example 27

### In Vivo Tumor Suppression of Synergistic Magnetic and Active Tumor-Targeted Delivery of Dox.

The U87MG tumor-bearing mice were randomly divided into six groups with 5 mice in each group. The first group of mice received PBS, as control group; the second group was injected with Dox solution, as “Dox” group; the third group was injected with Dox-MuMVs without magnetic attraction, as “magnet- RGD-” group; the fourth group was injected with RGD-Dox-MuMVs without magnetic enrichment, as “magnet-RGD+” group; the fifth group was injected with Dox-MuMVs under magnetic attraction, as “magnet+RGD-” group; the sixth group was injected with RGD-Dox-MuMVs under magnetic attraction, as “magnet+RGD+magnet” group. All the experimental groups (2-6 groups) are dispersed in 150 μl PBS before intravenous administration with a Dox-equivalent dose of 5 mg/kg. For the groups under magnetic attraction, the magnet was applied for 1 h along with injection of therapeutic agents. During half a month after the corresponding treatments, the volume of tumors was measured every other day and calculated by the following equation: V=L×W²/2. The body weight of the mice was also evaluated during this period to reveal the systemic toxicity of the delivery platform.

### Example 28

### Evaluation of Mice Survival.

All experiments with live animals were conducted in accordance with a protocol approved by the National Institutes of Health Animal Care and Use Committee (NIHACUC). In general, the mice must be euthanized when the tumor size reaches 2 cm, so the mice survival was evaluated based on the life span from the date when the mice received treatment to the date when the tumor size reached 2 cm. For each group subjected to the corresponding treatment, the survival rate was calculated by dividing the number of surviving mice at different days of post-treatment by the total number of mice before treatment.

Having now fully described this invention, it will be understood by those of ordinary skill in the art that the same can be performed within a wide and equivalent range of conditions, formulations and other parameters without affecting the scope of the invention or any embodiment thereof.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

All patents and publications cited herein are fully incorporated by reference herein in their entirety.

What is claimed is:

1. A composition comprising:
(a) a first block copolymer comprising at least two polymer blocks, wherein at least one of the polymer blocks has been functionalized;
(b) a plurality of first inorganic nanoparticles bound to the surface of the first block copolymer;
(c) a second block copolymer comprising at least two polymer blocks; and
(d) a plurality of second inorganic nanoparticles; or
(a′) a first block copolymer comprising at least two polymer blocks, wherein at least one of the polymer blocks has been functionalized;
(b′) a plurality of small molecules bound to the surface of the first block copolymer;
(c′) a second block copolymer comprising at least two polymer blocks; and
(d′) a plurality of inorganic nanoparticles, wherein the plurality of small molecules are bound to the surface of the inorganic nanoparticles;
wherein the composition is in the form of vesicles.

2. The composition of claim 1, wherein the first block copolymer in (a) or (a′) comprises a first polymer block and a second polymer block.

3. The composition of claim 2, wherein the first polymer block is polystyrene.

4. The composition of claim 2, wherein the second polymer block is poly(ethylene oxide).

5. The composition of claim 1, wherein the second block copolymer in (c) or (c′) comprises a first polymer block and a second polymer block.

6. The composition of claim 5, wherein the first polymer block is polystyrene.

7. The composition of claim 5, wherein the second polymer block in poly(acrylic acid).

8. The composition of claim 1, wherein the composition comprises:
(a) a first block copolymer comprising at least two polymer blocks, wherein at least one of the polymer blocks has been functionalized;
(b) a plurality of first inorganic nanoparticles bound to the surface of the first block copolymer;
(c) a second block copolymer comprising at least two polymer blocks; and
(d) a plurality of second inorganic nanoparticles;
wherein the composition is in the form of vesicles.

9. The composition of claim 8, wherein the first inorganic nanoparticles comprise Au.

10. The composition of claim 8, wherein the second inorganic nanoparticles comprise iron oxide.

11. The composition of claim 8, wherein the first block copolymer comprises a first polymer block comprising polystyrene and a second polymer block comprising poly(ethylene oxide), the first inorganic nanoparticles comprise Au having a diameter of from 20 nm to 50 nm, the second block copolymer comprises a first polymer block comprising polystyrene and a second polymer block comprising poly(acrylic acid), and the second inorganic nanoparticles comprise iron oxide.

12. The composition of claim 1, wherein the composition comprises:
(a′) a first block copolymer comprising at least two polymer blocks, wherein at least one of the polymer blocks has been functionalized;
(b′) a plurality of small molecules bound to the surface of the first block copolymer;
(c′) a second block copolymer comprising at least two polymer blocks; and
(d′) a plurality of inorganic nanoparticles, wherein the plurality of small molecules are bound to the surface of the inorganic nanoparticles;
wherein the composition is in the form of vesicles.

13. The composition of claim 12, wherein the small molecule comprises dopamine.

14. The composition of claim 12, wherein the inorganic nanoparticles comprise iron oxide.

15. The composition of claim 12, wherein the first block copolymer comprises a first polymer block comprising polystyrene and a second polymer block comprising poly(ethylene oxide), the small molecule is dopamine, the second polymer block copolymer comprises a first block comprising polystyrene and a second polymer block comprising poly(acrylic acid), and the inorganic nanoparticles comprise iron oxide.

16. The composition of claim 1, wherein the vesicles have a size range of 10 nm to 1000 nm.

17. The composition of claim 1, further comprising a therapeutic agent.

18. The composition of claim 1, wherein the therapeutic agent comprises doxorubicin.

19. The composition of claim 1, wherein the transverse relaxivity rate (r₂) of the formed vesicles is between about 150 mM⁻¹s⁻¹ to about 300 mM⁻¹s⁻¹.

20. A method of making a composition for delivery of a therapeutic agent, the method comprising:
(i) providing a composition in the form of vesicles comprising:
(a) a first block copolymer comprising at least two polymer blocks, wherein at least one of the polymer blocks has been functionalized;
(b) a plurality of first inorganic nanoparticles bound to the surface of the first block copolymer;
(c) a second block copolymer comprising at least two polymer blocks; and
(d) a plurality of second inorganic nanoparticles; or
(a′) a first block copolymer comprising at least two polymer blocks, wherein at least one of the polymer blocks has been functionalized;
(b′) a plurality of small molecules bound to the surface of the first block copolymer;
(c′) a second block copolymer comprising at least two polymer blocks; and
(d′) a plurality of inorganic nanoparticles, wherein the plurality of small molecules are bound to the surface of the inorganic nanoparticles; and
(ii) contacting the composition of (a) with a solution containing the therapeutic agent to be delivered and forming vesicles comprising the therapeutic agent encapsulated in the vesicles, thereby forming a composition in the form of vesicles for the delivery of the therapeutic agent.

21. A method of imaging a biological target, the method comprising:
(i) providing a composition in the form of vesicles comprising:
(a) a first block copolymer comprising at least two polymer blocks, wherein at least one of the polymer blocks has been functionalized;
(b) a plurality of first inorganic nanoparticles bound to the surface of the first block copolymer;
(c) a second block copolymer comprising at least two polymer blocks; and
(d) a plurality of second inorganic nanoparticles; or
(a′) a first block copolymer comprising at least two polymer blocks, wherein at least one of the polymer blocks has been functionalized;
(b′) a plurality of small molecules bound to the surface of the first block copolymer;
(c′) a second block copolymer comprising at least two polymer blocks; and
(d′) a plurality of inorganic nanoparticles, wherein the plurality of small molecules are bound to the surface of the inorganic nanoparticles; and
(ii) detecting the vesicles.

22. The method of claim 21, wherein detecting the vesicles uses one or more of a fluorescence microscope, laser-confocal microscopy, cross-polarization microscopy, nuclear scintigraphy, positron emission tomography, single photon emission computed tomography, magnetic resonance imaging, photoacoustic imaging, magnetic resonance spectroscopy, computed tomography, or a combination thereof.

23. The method of claim 21, wherein the formed vesicles in (i) have a transverse relaxivity (r₂) between about 150 mM⁻¹s⁻¹ to about 300 mM⁻¹s⁻¹.
